(19) United States
(12) Patent Application Publication
(10) Pub. No.: US 2020/0230207 A1
(43) Pub. Date: Jul. 23, 2020

(54) TREATMENT OF BONE GROWTH DISORDERS

(71) Applicant: FONDAZIONE TELETHON, Roma (IT)

(72) Inventors: Carmine SETTEMBRE, Pozzuoli (NA) (IT); Laura CINQUE, Pozzuoli (NA) (IT); Rosa BARTOLOMEO, Pozzuoli (NA) (IT); Alberto AURICCHIO, Pozzuoli (NA) (IT); Ivana TRAPANI, Pozzuoli (NA) (IT); Elisabetta TORIELLO, Pozzuoli (NA) (IT)

(21) Appl. No.: 15/763,155

(22) PCT Filed: Sep. 28, 2016
(86) PCT No.: PCT/EP2016/073149

§ 371 Date: Mar. 26, 2018

Related U.S. Application Data
(60) Provisional application No. 62/233,687, filed on Sep. 28, 2015.

(51) Int. Cl. A61K 38/17; A61P 19/08; A61K 45/06; A61K 31/4745

(52) U.S. Cl. CPC A61K 38/1761; A61P 19/08; A61K 48/00; A61K 31/4745; A61K 45/06

(57) ABSTRACT

The present invention relates to an activator of beclin 1-Vps 34 complex for use in the treatment and/or prevention of a bone growth disorder. The activator may be a polypeptide, a polynucleotide, a vector, a host cell or a small molecule. In particular the activator may be a Beclin 1 peptide or a fragment or a derivative thereof, a mTORC1 inhibitor or a BH3 mimetic. The present invention also relates to pharmaceutical composition comprising said activator.

### FIELD OF THE INVENTION

The present invention relates to an activator of beclin 1-Vps 34 complex for use in the treatment and/or prevention of a bone growth disorder. The activator may be a polypeptide, a polynucleotide, a vector, a host cell or a small molecule. In particular the activator may be a Beclin 1 peptide or a fragment or a derivative thereof, a mTORC1 inhibitor or a BH3 mimetic. The present invention also relates to pharmaceutical composition comprising said activator.

### BACKGROUND

Bones in different parts of the skeleton develop through two distinct processes, intramembranous ossification and endochondral ossification. Intramembranous ossification occurs in the flat bones of the skull and involves direct differentiation of embryonic mesenchymal cells into the bone-forming osteoblasts. Endochondral ossification is responsible for the initial bone development from cartilage, in utero and infants; furthermore it is an essential process during formation of long bones, for the longitudinal growth of long bones and for the natural healing of bone fractures.

Endochondral ossification begins when mesenchymal cells differentiate into chondrocytes, which secrete the various components of cartilage extracellular matrix (ECM), including collagen type II and the proteoglycan aggrecan, and which form a cartilage template for future bone. Ossification of the cartilage model is preceded by chondrocytes proliferation and hypertrophy. The primary centre of ossification, wherein blood vessels, osteoclasts, bone marrow and osteoblast precursors invade the model, expands towards the ends of the cartilage model, as the osteoclasts remove cartilage ECM and osteoblasts deposit bone on cartilage remnants. In long bones, a secondary ossification centre subsequently forms at each end of the cartilage model, leaving a cartilaginous growth plate between the primary and secondary ossification centres. Chondrocytes arranged into columns form the growth plate.

The growth plate (also called epiphyseal plate or physis) is a hyaline cartilage plate in the metaphysis at each end of a long bone. The plate is found in children and adolescents; in adults, who have stopped growing, the growth plate is replaced by the epiphyseal line. The growth plate is responsible for longitudinal growth of bones. Skeletal maturity occurs when the expanding primary centre of ossification meets the secondary centre of ossification.

Chondrocyte's rate of proliferation, hypertrophic differentiation and extracellular matrix (ECM) deposition in the growth plates mediate bone elongation.

Collagens are major structural components of the ECM. Type II collagen (Col2), also called cartilage collagen, is the major collagen synthesized by chondrocytes.

Type II collagen is comprised of 3 alpha-1(II) chains. These are synthesized in the chondrocytes of the growth plate as larger procollagen (PC2) chains, which contain N- and C-terminal amino acid sequences called pro-peptides. After secretion into the extracellular matrix, the pro-peptides are cleaved, forming the mature type II collagen molecule.

As the hypertrophic chondrocytes degenerate, osteoblasts ossify the remains to form new bone. Thus, the growth plate chondrocyte plays multiple important roles during its lifespan. It constructs the transient growth plate tissue, which has the necessary capacity to move in space through continued self-renewal and localized degradation, but simultaneously maintains the mechanical stability of the growing bone.

Defects in the development and maintenance of the growth plates lead to disorders of the bone growth.

Several bone diseases are associated to defects of the collagens, in particular of type II collagen, in particular those due to mutations of COL2A1 gene, coding for the pro-alpha chain of type II collagen (Kuivaniemi et al., 1997). Diseases associated to defects of type II collagen include: achondrogenesis Type II (due to mutation in the type II procollagen gene, leading to abnormal pro-alpha-1(II) chain and impaired assembly and/or folding of type II collagen), platyspondylic skeletal dysplasia, Torrance type, Hypochondrogenesis, Spondyloepiphyseal Dysplasia Congenita (SED), Spondylometaphyseal dysplasia (SMD), Kniest Dysplasia, Stickler Syndrome, Type I, Osteoarthritis Associated with Chondrodysplasia, Avascular Necrosis of the Femoral Head and Legg-Calve-Perthes Disease, Otospondylomegaepiphyseal Dysplasia, Strudwick type of spondyloepimetaphyseal dysplasia, Multiple epiphyseal dysplasia with myopia and conductive deafness, Spondyloperipheral dysplasia, Czech dysplasia.

The most common bone growth disorder is achondroplasia. Achondroplasia is the most common cause of dwarfism. Achondroplasia family is characterized by a continuum of severity ranging from mild (hypochondroplasia, HCH; OMIM:146000) and more severe forms (achondroplasia) to lethal neonatal dwarfism (thanatophoric dysplasia, TD; OMIM:187600). The condition occurs in 1 in 15,000 to 40,000 newborns. Affected individuals exhibit short stature caused by rhizomelic shortening of the limbs, characteristic facies with frontal bossing and midface hypoplasia, exaggerated lumbar lordosis, limitation of elbow extension, genu varum, and trident hand.

Two specific mutations in the FGFR3 gene are responsible for almost all cases of achondroplasia. These mutations cause the FGFR3 protein to be overly active, which interferes with skeletal development and leads to the disturbances in bone growth seen with this disorder.

Dominant mutations in the FGFR3 gene affect predominantly bones that develop by endochondral ossification, whereas dominant mutations involving FGFR1 (OMIM:136350) and FGFR2 (OMIM:176943) principally cause syndromes that involve bones arising by membranous ossification.

Other FGFR3 associated diseases include: thanatophoric dysplasia types 1 and 2 and SADDAN (severe achondroplasia-developmental delay-acanthosis nigricans).

Hypochondroplasia is a form of short-limbed dwarfism. This condition affects the conversion of cartilage into bone (a process called ossification), particularly in the long bones of the arms and legs. Hypochondroplasia is similar to achondroplasia, but the features tend to be milder. About 70 percent of all cases of hypochondroplasia are caused by mutations in the FGFR3 gene. The incidence of hypochondroplasia is unknown. Researchers believe that it may be about as common as achondroplasia, which occurs in 1 in 15,000 to 40,000 newborns. More than 200 people worldwide have been diagnosed with hypochondroplasia.

Evidences indicate that activated FGFR3 is targeted for lysosomal degradation and that activating mutations found in patients with achondroplasia and related chondrodysplasias disturb this process, leading to recycling of activated receptors and amplification of FGFR3 signals (Cho et al., 2004).

Fibroblastic growth factors (FGF) are a family of polypeptides that are involved in numerous developmental processes including embryonic and skeletal development. The function of FGFs is dependent on the spatial and temporal expression of FGF receptors.

FGF18 is an important mediator for skeletal development. Murine Fgf18 binds primarily to FGFR3; furthermore, it binds to FGFR1 in chondrocytes. Inhibition of chondrocyte proliferation and differentiation by FGF18 stimulation in embryos has been previously reported (Kapadia et al., 2005). Further studies indicate that FGF18 positively regulates osteogenesis and negatively regulates chondrogenesis (Ohbayashi, 2002). The activation of FGFR3 has been reported to inhibit the proliferation and differentiation of growth plate chondrocytes (Naski et al., 1998). On the contrary, FGF18 has been shown to have positive effects on chondrocytes in other cartilaginous tissues apart from the growth plate and it has recently been shown that intra articular injection of FGF18 can stimulate the repair of damaged cartilage in a rat model of osteoarthritis (Moore et al., 2005).

Both FGFR3 and FGF18 knockout mice reveal the same phenotype of long bones during embryonic development. All Fgf18^{-/-} mice express skeletal abnormalities including curved radius and tibia and some mice show incomplete development of the fibula. Embryos are approximately 10-15% smaller than the wild type (Liu et al., 2002). The length of the long bone however is considerably smaller in FGF18^{-/-} mice, in comparison to the wild-type, than for FGFR3^{-/-} mice. This difference implies that other signaling pathways, such as FGF18 interaction with other FGF receptors, may be involved in osteogenesis of developing long bone (Ohbayashi et al., 2002).

A genome wide association study, showing that FGFR4 sequence variations may influence human height is described by Lango Allen, H. et al.

Defects in the bone growth are also associated to several Lysosomal storage disorders (LSDs).

Lysosomal storage disorders affect multiple organs including the skeleton. LSDs are a group of approximately 70 inherited diseases characterized by lysosomal dysfunction and neurodegeneration. Although individually rare, the lysosomal storage disorders (LSDs) as a group have a frequency of about 1:8000 live births, making this disease group a major challenge for the health care system. So far, mutations in more than 20 genes encoding for lysosomal proteins cause defects in bone growth and development.

LSDs with prominent skeletal symptoms include type 1 and type 3 Gaucher disease, the mucopolysaccharidoses, multiple sulfatase deficiency, mucolipidosis type II and III, galactosidosis, mannosidosis (alpha and beta), fucosidosis and pycnodysostosis (Clarke and Hollak, 2015).

The mucopolysaccharidosis (MPS) syndromes are lysosomal storage diseases with an overall incidence of about 1:25000. Skeletal manifestations are often the presenting symptom(s) for patients with MPS I, II, IV, VI, VII and IX. Disease symptoms include alteration of linear bone growth, morphologic abnormalities of bone shape and structural as well as functional abnormalities in articular cartilage. Alteration of linear bone growth leading to proportionate short stature is a characteristic feature of all severely affected MPS I, II, IV, VI and VII patients, who show relatively normal linear growth in the first 18 months of life followed by a period of impaired growth with little or no further growth after the age of 8 years.

Hurler and Scheie syndromes represent phenotypes at the severe and mild ends of the MPS I clinical spectrum, respectively, and the Hurler-Scheie syndrome is intermediate in phenotypic expression. Length is often normal until about 2 years of age when growth stops; by age 3 years height is less than the third percentile. The long tubular bones show diaphyseal widening with small, deformed epiphyses. Phalanges are bullet-shaped with proximal pointing of the second to fifth metacarpals. Hurler syndrome is characterized by skeletal abnormalities, cognitive impairment, heart disease, respiratory problems, enlarged liver and spleen, characteristic facies and reduced life expectancy. The prevalence of the Hurler subtype of MPS 1 is estimated at 1/200,000 in Europe. Scheie syndrome is characterized by skeletal deformities and a delay in motor development. Prevalence of Scheie syndrome is estimated at 1/500,000.

Mucopolysaccharidosis type 2 (MPS 2) is a lysosomal storage disease leading to a massive accumulation of glycosaminoglycans and a wide variety of symptoms including distinctive coarse facial features, short stature, cardio-respiratory involvement and skeletal abnormalities. It manifests as a continuum varying from a severe to an attenuated form without neuronal involvement. Prevalence at birth in Europe is 1/166,000. It is an X-linked recessive disorder; very rare cases of female presentation have been reported.

Mucopolysaccharidosis type 4 (MPS IV) is a lysosomal storage disease belonging to the group of mucopolysaccharidoses, and characterised by spondylo-epiphyso-metaphyseal dysplasia. It exists in two forms, A and B. Prevalence is approximately 1:250000 for type WA but incidence varies widely between countries. MPS IVB is even rarer. MPS IVA is characterized by intracellular accumulation of keratan sulfate and chondroitin-6-sulfate. Key clinical features include short stature, skeletal dysplasia, dental anomalies, and corneal clouding.

Mucopolysaccharidosis type 6 (MPS VI) is a lysosomal storage disease with progressive multisystem involvement, associated with a deficiency of arylsulfatase B (ASB or ARSB) leading to the accumulation of dermatan sulfate. Birth prevalence is between 1 in 43,261 and 1 in 1,505,160 live births. Prevalence: 1-9/100000. Mucopolysaccharidosis type VI results from a deficiency of arylsulfatase B. Clinical features and severity are variable, but usually include short stature, hepatosplenomegaly, dysostosis multiplex, stiff joints, corneal clouding, cardiac abnormalities, and facial dysmorphism. Intelligence is usually normal.

Mucopolysaccharidosis type 7 (MPS VII or Sly syndrome) is a very rare lysosomal storage disease belonging to the group of mucopolysaccharidoses, resulting from a deficiency of β-glucuronidase (GUSB). Less than 40 patients with neonatal to moderate presentation have been reported since the initial description of the disease by Sly in 1973. However, the frequency of the disease may be underestimated as the most frequent presentation is the antenatal form, which remains underdiagnosed. Prevalence is lower than 1:1,000,000. MPS VII is characterized by the inability to degrade glucuronic acid-containing glycosaminoglycans. The phenotype ranges from severe lethal hydrops fetalis to mild forms with survival into adulthood. Most patients with the intermediate phenotype show hepatomegaly, skeletal anomalies, coarse facies, and variable degrees of mental impairment. Currently, MPS VII lacks an efficient treatment.

Multiple sulfatase deficiency (MSD) is an autosomal recessive inborn error of metabolism resulting in tissue accumulation of sulfatides, sulfated glycosaminoglycans, sphingolipids, and steroid sulfates. The enzymatic defect affects the whole family of sulfatase enzymes; thus, the disorder combines features of metachromatic leukodystrophy and of various mucopolysaccharidoses. Affected individuals show neurologic deterioration with mental retardation, skeletal anomalies, organomegaly, and ichthyosis.

Gaucher disease (GD) is a lysosomal storage disorder encompassing three main forms (types 1, 2 and 3), a fetal form and a variant with cardiac involvement. The prevalence is approximately 1/100,000. GD type 1 (90% of cases) is the chronic and non-neurological form associated with organomegaly (spleen, liver), bone anomalies (pain, osteonecrosis, pathological fractures) and cytopenia. GD is due to mutations in the GBA gene (1q21) that codes for a lysosomal enzyme, glucocerebrosidase, or in very rare cases the PSAP gene that codes for its activator protein (saposin C). The deficiency in glucocerebrosidase leads to the accumulation of glucosylceramidase (or beta-glucocerebrosidase) deposits in the cells of the reticuloendothelial system of the liver, the spleen and the bone marrow (Gaucher cells). Formal diagnosis of the disease is determined by the measurement of glucocerebrosidase levels in circulating leukocytes. Genotyping confirms the diagnosis.

Current treatments for LSDs are enzyme replacement therapy, substrate reduction therapy and hematopoietic stem cell transplantation. However, effects of these interventions on skeletal disease manifestations are less well established and outcomes are highly dependent on disease burden at treatment initiation. Furthermore, the efficacy of these therapeutic strategies has several major limitations, such as the difficulty of reaching particular tissues such as the skeleton. Indeed, gene therapy approaches in different MPS animal models showed very little efficacy on bone defects (Ferla R et al., 2014, Stevenson D A and Steiner R D, 2013).

Although orthopedic surgery and neurosurgery are important components of care for MPS patients this approach to therapy is largely symptomatic and thus does not alter the primary underlying skeletal pathology. Therapies directed towards the primary metabolic block that have been utilized in the MPSs include bone marrow transplantation and enzyme replacement therapy.

The main treatment option for short stature, e.g. in achondroplasia patients, is administration of recombinant growth hormone (rGH). Recently a phase II study started for evaluating the use of BMN 111, a 39 amino acid analog of C-type natriuretic peptide (CNP), for the treatment of achondroplasia.

Improved treatments that target skeletal diseases are however still needed.

The inventors have surprisingly identified dysregulation of endocytic trafficking and autophagy as a target for treating bone growth disorders.

Autophagy is an essential cellular process that consists of selective degradation of cellular components. There are at least three different types of autophagy described: macroautophagy (also referred to as autophagy), microautophagy and chaperone mediated autophagy. The initial step of autophagy is the surrounding and sequestering of cytoplasmic organelles and proteins within an isolation membrane (phagophore). Potential sources for the membrane to generate the phagophore include the Golgi complex, endosomes, the endoplasmic reticulum (ER), mitochondria and the plasma membrane (Kang et al., 2011).

The nascent membranes are fused at their edges to form double-membrane vesicles, called autophagosomes. Autophagosomes undergo a stepwise maturation process, including fusion with acidified endosomal and/or lysosomal vesicles, eventually leading to the delivery of cytoplasmic contents to lysosomal components, where they fuse, then degrade and are recycled.

Autophagy depends on Atg5/Atg7, it is associated with microtubule-associated protein light chain 3 (LC3) truncation and lipidation, and may originate directly from the ER membrane and other membrane organelles. Furthermore, recent study has identified a Atg5/Atg7-independent pathway of autophagy. This pathway of autophagy was not associated with LC3 processing but appeared to involve autophagosome formation from late endosomes and the trans-Golgi.

Beclin 1 (NP_003757) is the mammalian ortholog of yeast Atg6/Vps30 and it is required for Atg5/Atg7-dependent and -independent autophagy. It forms a protein complex with the class III phosphatidylinositol 3-kinase (PI3KC3)Vps34 (NP_001294949.1; NP_002638.2) and with Vps15 (NP_055417). Beclin 1 encodes a 450 amino acid protein with a central coiled coil domain. Within its N-terminus, it contains a BH3-only domain, which mediates binding to anti-apoptotic molecules such as Bcl-2 and Bcl-xL. The most highly conserved region, referred to as the evolutionarily conserved domain (ECD), spans from amino acids 244-337 and is important for its interaction with Vps34.

The Beclin 1/Vps34 complex (also known as class III phosphatidylinositol 3-kinase complex) is a multivalent trafficking effector that regulates autophagosome formation, including the nucleation of the phagophore at the endoplasmic reticulum (autophagic vesicle nucleation) and autophagosomes maturation.

Furthermore, the Beclin 1/Vps34 complex promotes endocytic trafficking (McKnight N C et al., 2014; Levine B et al., 2015).

Besides Vps15, the complex has numerous other binding partners, including Atg14L (another core autophagy protein), UVRAG (a protein that functions in autophagosomal maturation and endocytic maturation) and Ambral (a positive regulator of the Beclin 1/Vps34 complex). In addition, Beclin 1 has been reported to interact with certain receptors and immune signaling adaptor proteins, including the inositol 1, 4, 5-triphosphate receptor (IP3R), the estrogen receptor, MyD88 and TRIF, and nPIST, as well as certain viral virulence proteins such as HSV-1 ICP34, KSHV vBcl-2, HIV-1 Nef, and influenza M2. A further binding partner is Rubicon, which however is a negative regulator of the Beclin 1/Vps34 complex.

Activation of a Beclin 1/Vps34 complex thus induces autophagy in a cell and/or promotes endocytic trafficking.

Activators of Beclin 1/Vps34 complex stimulate Beclin 1-dependent lipid kinase activity of Vps34. Vps34 kinase activity upregulates the phosphatidylinositol 3-phosphates (PI3P) at the phagophore. Activators of Beclin 1/Vps34 complex increase PI3P production in a cell.

The mechanistic target of rapamycin, also known as mammalian target of rapamycin (mTOR), is a protein encoded in humans by the MTOR gene. mTOR is a serine/threonine protein kinase that regulates cell growth, cell proliferation, cell motility, cell survival, protein synthesis, autophagy, and transcription. mTOR belongs to the phosphatidylinositol 3-kinase-related kinase protein family and it is the catalytic subunit of two structurally distinct complexes: mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8) and the non-core components PRAS40 and DEPTOR. Upon inhibition, mTOR induces autophagy. In particular, mTORC1 inhibition. e.g. by amino acid starvation or pharmacological inhibition, leads to de-repression of ULK kinase activity. The active ULK directly phosphorylates Beclin-1 and activates Beclin 1-Vps34 complex.

An exemplary synthetic peptide capable of activating Beclin 1-Vps34 complex and called Tat-Beclin 1 peptide has been recently disclosed by Shoji-Kawata et al. (Nature 2013). Tat-Beclin 1 (also known as Atg6 Activator I, Beclin 1-GAPR-1 Interaction Blocker I, Vps30 Activator I, Autophagy Inducer IV) is a cell-permeable peptide that is composed of essential HIV-1 virulence factor Nef-binding sequence derived from human Atg6/Beclin 1 (aa 269-283) evolutionarily conserved domain (ECD) with substitutions at three non-species-conserved residues (H275E, S279D, and Q281E) for enhanced solubility and N-terminally fused to the membrane-permeant HIV-1 Tat protein transduction domain (PTD) sequence (aa 47-57) via a-Gly-Gly-linkage, to facilitate cellular delivery and Beclin 1 activation via competitive binding to its negative regulator “Golgi-associated plant pathogenesis-related protein-1” (GAPR-1/GLIPR2) on the Golgi surface. Tat-Beclin 1 peptide induces a complete cellular autophagy response. Tat-Beclin 1 peptide may promote the release of Beclin 1 from the Golgi, resulting in enhanced early autophagosome formation. Other unknown mechanisms may also contribute to the Beclin 1-Vps 34 complex activation and autophagy induction accomplished by Tat-Beclin 1.

Tat-Beclin 1 peptide treatment in multiple cell lines (e.g. HeLa, COS-7, MEFs, A549, HBEC30-KT, THP1, and HCC827 cells) leads to p62 degradation and LC3-II conversion.

Phosphatidylethanolamine (PE) conjugation of mammalian LC3 results in a non-soluble form of LC3 (LC3-II) that stably associates with the autophagosomal membrane. Lipidated LC3 (LC3-II), but not unlipidated LC3 (LC3-I), binds to autophagosomes and LC3 lipidation correlates with autophagosome formation. When autophagy is induced, western blot analysis reveals that LC3-II protein levels are increased.

p62 protein is selectively degraded by the autophagy machinery and its protein levels reflects the amount of autophagic flux (i.e. a complete autophagy response). When autophagy is induced, western blot analysis reveals that p62 protein levels are decreased.

Also a retro-inverso Tat-Beclin 1 peptide has been disclosed (Shoji-Kawata et al., 2013), which is capable of activating Beclin 1/Vps34 complex: the retro-inverso Tat-Beclin 1 peptide (also known as Atg6 Activator II, Beclin-1-GAPR-1 Interaction Blocker II, Vps30 Activator II), consists in the all-D-amino acid retro-inverso sequence of Tat-Beclin 1.

Administration to mice of any of the two peptides leads to increase of autophagosomes in peripheral tissues (skeletal muscles and cardial muscles, pancreas, at 20 mg/kg i.p.) and increase of autophagosomes in central nervous system of neonatal mice (15 mg/kg, 1/die for 2 weeks). Daily treatment with Tat-Beclin 1 peptides for 2 weeks in adult and neonatal mice is well-tolerated. Efficient reduction of infections in mice infected with CHKN (muscle, skin, joints) or WNV (CNS) consequent to administration of Tat-Beclin 1 peptide is also shown by Shoji-Kawata et al. (Nature, 2013). No further therapeutic effects, nor activity, of a Tat-Beclin 1 peptide or derivatives thereof have ever been shown in skeletal tissue.

Beclin 1 peptide analogues, fragments or derivatives thereof, such as Tat-Beclin 1 peptide, are disclosed in WO2013119377 and WO2014149440 incorporated by reference. The use of said peptides, analogues, fragments or derivatives thereof for the treatment of bone-related disorders has never been disclosed nor suggested.

WO2011106684 discloses Beclin-1 derivative peptides of sequence comprising all or a subsequence of Beclin 1, fused to the protein transduction domain of an HIV Tat protein. WO2011106684 generally refers to the use of autophagy modulators for treating diseases with dysregulated autophagy. Among others, lysosomal storage disorders are mentioned, however a direct correlation between the use of an autophagy inducer or of Beclin-1 derivative peptides and the treatment of lysosomal storage disorders is not disclosed.

WO201128941 claims methods of treating lysosomal storage disease through inhibition of autophagy.

Shapiro et al (Autophagy, 2014) disclose that patients and mouse models of LSDs display a higher number of autophagosomes, most likely resulting from a defective lysosome-autophagosome fusion. Furthermore, it discloses that treatment of rats with the autophagy activator rapamycin impairs longitudinal growth.

Alvarez-Garcia et al. (Pediatr Nephrol, 2007) disclose that rapamycin impairs longitudinal growth in young rats, causing marked alterations in the growth plate, and that rapamycin disrupts angiogenesis and decreases proliferation and hypertrophy of growth cartilage chondrocytes. In humans, Gonzalez et al (Pediatr Nephrol, 2010) disclose lower growth rate in a small series of kidney transplanted children treated with rapamycin in comparison with a control group not treated with rapamycin.

Settembre et al. (Autophagy, 2009) disclose that autophagy is important for chondrocyte metabolism during endochondral ossification, and also hypothesize that its impairment may contribute to the development of skeletal abnormalities, such as those observed in MSD. However, they do not provide any evidence or suggestion that induction of autophagy and in particular that activation of the Beclin-1/Vps34 complex could be effective in the treatment of bone disorders.

The inventors have unexpectedly shown that alterations of the autophagic cellular function primary lead to bone growth disorders.

Surprisingly, molecules that are capable of activating Beclin-1/Vps34 complex, which is involved in initiation of the autophagic pathway and in the regulation of endocytic vesicles trafficking, efficiently prevent and/or treat bone growth pathologies.

### SUMMARY OF THE INVENTION

The present invention provides an activator of beclin 1-Vps 34 complex for use in the treatment and/or prevention of a bone growth disorder wherein said activator is selected from the group consisting of:
a) a polypeptide comprising a Beclin 1 peptide consisting of SEQ ID No. 43 or a functional fragment thereof or a functional derivative thereof;
b) a polynucleotide coding for said polypeptide;
c) a vector comprising said polynucleotide;
d) a host cell expressing said polypeptide or said polynucleotide;
e) a small molecule selected from the group of a mTORC1 inhibitor or a BH3 mimetic.

Preferably the activator increases phosphatidylinositol 3-phosphates (PI3P) production in a cell.

Preferably the functional fragment comprises residues 270-278 of SEQ ID No. 43. In the present invention the functional derivatives may be functional derivatives of SEQ ID No. 43 or of a functional fragment thereof. For instance the functional derivatives may be the derivative of a functional fragment comprising residues 270-278 of SEQ ID No. 43. Functional derivatives are defined below.

Yet preferably the functional fragment is flanked by no more than twelve naturally-flanking Beclin 1 residues. This means that on each sides (at N and C terminal of residues 270-278 of SEQ ID NO:43) a maximum of 12 amino acids can be present. Such amino acid may be the same amino acid present in Beclin 1 in these positions (i.e. “naturally-flanking” Beclin 1 residues).

Preferably the functional derivative comprises SEQ ID NO: 43 or a functional fragment thereof and wherein said functional derivative comprises from 1 to 6 amino acid residue substitution(s) and/or a heterologous moiety.

Preferably the heterologous moiety consists of SEQ ID No. 44 or SEQ ID No. 45.

Preferably the polypeptide or the functional fragment thereof or the functional derivative thereof is partially or fully cyclized.

In a preferred embodiment the polypeptide is a retro-inverso polypeptide.

Still preferably the polypeptide comprises a sequence selected from the group consisting of: SEQ ID No. 1, SEQ ID No. 2, SEQ ID No. 12 to SEQ ID No. 38 or a functional fragment thereof or a functional derivative thereof.

In a preferred embodiment the activator is a polynucleotide encoding for the polypeptide as defined in any of claims 3 to 9, preferably the polynucleotide comprises SEQ ID NO: 7.

In a preferred embodiment the activator is a vector comprising the polynucleotide as defined above, preferably said vector is a viral vector.

Preferably the activator further comprises a polynucleotide coding for the wild-type form of the protein whose mutated form is responsible for the bone growth disorder or a vector comprising said polynucleotide or further comprising the wild-type form of a protein whose mutated form is responsible for the bone growth disorder.

Preferably the protein whose mutated form is responsible for the bone growth disorder is selected from the group consisting of: FGFR3, FGFR1, FGFR2, FGFR4, β-glucocerebrosidase, α-mannosidase, α-fucosidase, α-neuraminidase, Cathepsin-A, UDP-N-acetylglucosamine, N-acetylglucosamine-1-phosphotransferase, Sulfatase modifying factor 1, Cathepsin K, α-L-iduronidase, Iduronate-2-sulfatase, Heparan N-sulfatase, α-N-acetyl glucosaminidase, Acetyl-CoA: α-glucosaminide acetyltransferase, N-acetylglucosamine 6-sulfatase, N-acetylgalactosamine-6-sulfatase, β-D-galactosidase, N-acetylgalactosamine-4-sulfatase, β-glucuronidase and Hyaluronidase.

In a preferred embodiment the inhibitor of mTORC1 is selected from the group consisting of: Rapamycin, KU0063794, WYE354, Deforolimus, TORN 1, TORN 2, Temsirolimus, Everolimus, sirolimus, NVP-BEZ235 and PI103.

In a yet preferred embodiment the bone growth disorder is selected from the group consisting of: achondroplasia, hypochondroplasia, spondyloepiphyseal dysplasia, a lysosomal storage disorder, preferably a mucopolysaccharidosis (MPS).

Preferably the lysosomal storage disorder is selected from the group consisting of: MPS I, MPS II, MPS IV, MPS VI, MPS VII, MPS IX, Gaucher disease type 3, Gaucher disease type 1, multiple sulfatase deficiency, mucolipidosis type II, mucolipidosis type III, galactosidosis, alpha-mannosidosis, beta-mannosidosis, fucosidosis, pycnodysostosis.

Still preferably the bone growth disorder is selected from the group consisting of: achondroplasia, MPS VI and MPS VII.

The present invention also provides a pharmaceutical composition for use in the treatment and/or prevention of a bone growth disorder comprising the activator as defined above and pharmaceutically acceptable carriers.

Preferably the pharmaceutical composition further comprises a polynucleotide coding for the wild-type form of the protein whose mutated form is responsible for the bone growth disorder or a vector comprising said polynucleotide or further comprising the wild-type form of a protein whose mutated form is responsible for the bone growth disorder.

Preferably the pharmaceutical composition further comprises a therapeutic agent, preferably the therapeutic agent is selected from: enzyme replacement therapy, growth hormone, BMN111.

The present invention also provides a method for the treatment and/or prevention of a bone growth disorder in a subject in need thereof comprising administering an effective amount of the activator as defined above or the pharmaceutical composition as defined above.

The present invention also provides a vector for use in the treatment and/or prevention of a bone growth disorder said vector comprising a polynucleotide coding for an activator of beclin 1-Vps 34 complex, wherein said activator of beclin 1-Vps 34 complex is a polypeptide comprising a Beclin 1 peptide consisting of SEQ ID No. 43 or a functional fragment thereof or a functional derivative thereof, preferably, the functional fragment comprises residues 270-278 of SEQ ID No. 43, preferably the functional derivative comprises SEQ ID NO: 43 or a functional fragment thereof and said functional derivative comprises from 1 to 6 amino acid residue substitution(s) and/or a heterologous moiety.

Preferably the polynucleotide encodes a peptide consisting of a sequence selected from the group consisting of: SEQ ID No. 1, SEQ ID No. 2, SEQ ID No. 12 to SEQ ID No. 38 or a functional fragment thereof or a functional derivative thereof.

Still preferably the polynucleotide comprises SEQ ID No. 3, preferably it is a viral vector, preferably an adeno-associated vector (AAV).

Yet preferably the vector further comprises a polynucleotide coding for the wild-type form of the protein whose mutated form is responsible for a bone growth disorder.

According to a preferred embodiment, the activator of the invention is a peptide comprising the sequence YGRKKRRQRRRGGTNVFNATFEIWHDGEFGT (SEQ ID NO: 1, herein Tat-Beclin 1 peptide), or a functional fragment or a functional derivative thereof.

According to a preferred embodiment, the activator of the invention is a peptide comprising the sequence RRRQRRKKRGYGGTGFEGDHWIEFTANFVNT (SEQ ID NO: 2, herein retro-inverso Tat-Beclin 1 or (D)-Tat-Beclin 1) or a functional fragment or a functional derivative thereof.

In the present invention functional fragments of SEQ ID No. 43 and functional derivatives maintain the biological activity of increasing phosphatidylinositol-3-phosphates production, which can be easily measured by methods known in the art.

### BRIEF DESCRIPTION OF FIGURES

FIG. 1: A, Representative images of p62, GFP-LC3 puncta (autophagosomes) and Lamp-1 immunostaining in femoral growth plates from Gusb^{-/-}; GFP-LC3^{tg/+} mice at P6. Tat-beclin-1 peptide was administered intraperitoneally (i.p.) where indicated (2 mg kg⁻¹, daily for 6 days). The insets show a higher magnification of co-localization in selected areas. Scale bar, 10 μm. B, Quantification of Lamp-1-LC3. Values are Mander's coefficients means (±s.e.m.) of n=3 mice per group (Student's t-test, *p<0.05).

FIG. 2: Analysis of femur and tibia lengths in MPSVII and MPSVI mice treated with Tat-Beclin 1 peptide, according to a preferred embodiment of the invention, compared to not treated mice. Femur and tibia mean lengths from wild-type (WT) and MPSVII mice at P15 and P30 (A), and from WT and MPSVI mice at P15 (B), treated with Tat-Beclin 1 where indicated (values represent mean±sem. Student t-test ***p<0,0005; **p<0,005; *p<0.05. At least 6 mice/genotype were analyzed). (C) Alizarin red/alcian blue staining of femurs and Tibia isolated from GUSB+/+(WT), GUSB−/− (MPSVII) and GUSB−/−; TAT-Beclin 1 mice at P15. (D) Representative images of alcian blue/alizared red staining of femurs and tibias from P15 Arsb^{+/±}(WT), Arsb^{-/-} (MPS VI) and Arsb^{-/-} Tat-Beclin 1 treated (MPS VI+Tat-beclin 1, at 2 mg/kg daily for 15 days) mice (n≥6 mice per group).

FIG. 3: Histological analysis of MPSVII mice growth plates treated with Tat-Beclin 1 peptide, according to a preferred embodiment of the invention, compared to not treated mice. A, H/E staining of tibial section from P15 WT, MPSVII and Tat-Beclin 1 injected MPSVII mice. B, BrDU staining of tibial section from P15 WT, MPSVII and Tat-Beclin 1 injected MPSVII mice, showing reduced proliferation index in MPSVII mice and rescued phenotype in Tat-Beclin 1 injected MPSVII mice. Bar graph shows mean value of BrDU index in mice with indicated genotypes and treatments (values represent mean±sem. Student t-test *p<0.05. At least 3 mice/genotype were analyzed). C, Representative images of P15 femoral growth plates sections from WT, MPSVII and Tat-Beclin 1 injected MPSVII mice immunostained with Collagen X and Collagen II. Nuclei were counterstained with hematoxylin. N=3 mice per group. Scale bar (100 μm). D-E, Bar graphs displaying the length of hypertrophic zone measured according to Col1 X staining (D, ANOVA, P=0.002; Tukey's post-hoc test, * p<0.05, ** p<0.005) and the amount of collagen (% of WT, Gusb^{+/+}) in the growth plate homogenates (E, ANOVA, P=9.52E−05; Tukey's post-hoc test, *p<0.05, ***p<0.0005).

FIG. 4: A1, Western blot analysis of LC3 and p62 proteins in chondrosarcoma cell line (RCS) called Rx chondrocytes¹³ treated with vehicle, 10 and 20 μM Tat-Beclin 1 peptide (Millipore). β-actin was used as loading control. A, Western blot analysis of FGFR3 protein in RCS, FGFR3 WT, FGFR3^{ach} and FGFR3^{TD} chondrocytes. β-actin was used as loading control. b-c, Western blot analysis of LC3 protein in serum starved WT, FGFR3^{mil} and FGFR3^{TD} chondrocytes treated with bafilomycin (200 nM) (B) and with leupeptin (50 μM) (C) where indicated. Bars graphs represent LC3II quantification relative to β-actin. (values represent mean±sd. Student t-test *p<0,01. N=3 experiments). D, FACS analysis of endogenous LC3 fluorescence in serum starved WT, FGFR3^{ach} and FGFR3^{TD} chondrocytes treated with bafilomycin (200 nM) where indicated. Graphs show representative histograms.

FIG. 5: a, Representative images of GFP-LC3 puncta (autophagosomes) in femoral growth plates from GFP-LC3tg/+ transgenic mice at indicated ages. Scale bar 10 μm. The insets show a high magnification of selected areas. b, Quantification of data (mean±s.e.m of n=3 mice/group. ***p<0.0005 ANOVA with post-hoc test). c, Western blot analysis of LC3I/II (non-lipidated and lipidated forms of MAP1LC3, respectively) of femoral growth plates from mice at indicated ages. β-actin was used as a loading control. Quantification of data (mean±s.e.m of n=3 mice/group. **p<0.005; ***p<0.0005 ANOVA with posthoc test). d, Total collagen content in femoral cartilage of mice with indicated ages and genotypes. Values (mean±s.e.m of at least 3 mice/group) were normalized to total DNA and expressed as % relative to P0 control mice (Atg7 f/f). **p<0.005, ***p<0.0005 Student's t-test. e, Native collagen levels detected using Coomassie blue staining of pepsin-digested growth plate extracts from P6 mice with indicated genotype. M=marker. f, TEM of the inter-territorial matrix of the proliferating zone of femoral growth plates of mice with indicated genotypes at P6. Arrows indicate collagen fibers. Scale bar 200 nm·g, Confocal analysis of intracellular PC2 (Col2a1) deposits in growth plate chondrocytes from mice with indicated genotypes at P6. Scale bar 20 μm. Arrows indicate Col2a1 deposits. The graph represents number of cells with Col2a1 dots (%) (mean±s.e.m of at least 70 cells/mouse. N=4 mice/group; *p<0.05; ***p<0.0005 ANOVA with post-hoc test).

FIG. 6: a, b, Quantification of secreted PC2 in control and Spautin-1-treated (24 h, 50 μM) (a) and Atg7-knockdown (KD; b) RCS chondrocytes after ER block release of PC2 (min). Ctrl, control. Mean values (±standard deviation (s.d.)) of 3 independent experiments. ANOVA, P=5.29×10⁻⁵ (a), P=0.007 (b); Sidak post-hoc test, *1³<0.05, ***P<0.0005. c, PC2 localization in Golgi area and ER (HSP47) in vehicle and Spautin-1-treated (24 h, 50 μM) RCS chondrocytes 10 min after the ER block release of PC2. Bar graph represents the percentage (±s.d.) of cells containing PC2 in the indicated cellular compartment. N=60; n=3 independent experiments. Student's t-test, **P<0.005. Scale bar, 10 μm. d, Immunofluorescence of PC2, Sec31 and GFP-LC3 in RCS chondrocytes. The insets show higher magnification and single colour channels of the boxed area. N, nucleus. The data are representative of 5 independent experiments. Scale bar, 5 μm. e, Spinning-disk confocal image of RCS chondrocytes co-expressing GFP-LC3- and mCherry-PC2-tagged proteins. Arrows show PC2 molecules sequestered by GFP vesicles. The data are representative of 3 independent experiments. Scale bar, 10 μm. f, Time-lapse stills of PC2 and LC3 from the boxed region in e.

FIG. 7: a, Western blot analysis of LC3I/II, SQSTM1 (p62), PDI and GOLPH3 in protein extracts from femoral growth plates of E18.5 mice with the indicated genotypes. β-actin was used as a loading control. b, Western blot analysis of LC3I/II of femoral growth plates from control and fgfl 8+/− mice at indicated ages. β-actin was used as a loading control. c, Graph represents quantification of LC3II levels in growth plates of wild type and Fgf18+/− mice at the indicated ages. Values were normalized to P0 samples (mean±s.e.m. N=3/mice for time point. *p<0.05 ANOVA with post-hoc test). d, Western blot analysis of LC3II levels in fgfr1,2,3,4 kd Rx chondrocytes treated with FGF18. BafA1 was added (200 nM, 3h). Bar graphs represent mean values±s.e. of n=8 independent experiments. Student's t-test *p<0.05, **p<0.005. NS=not significant. Quantification of LC3 positive vesicles in Rx chondrocytes is shown below. Vesicles were counted in at least 40 cells/treatment. Values represent mean values±s.e.m of n=3 independent experiments; Student t-test ***p<0.0005. NS=not significant. e, Western blot analysis of LC3I/II, FGFR3 of femoral growth plates from control and Fgfr3−/− mice (N=3). β-actin was used as a loading control. f, Western blot analysis of LC3I/II, FGFR4 of femoral growth plates from control and Fgfr4−/− mice. β-actin was used as a loading control. Graph represents quantification (mean±s.e.m. N=3/mice) of LC3II levels relative to β-actin. Student t-test ***p<0.0005; NS=not significant.

FIG. 8: a, Representative images of GFP-LC3 puncta (autophagosomes) in femoral growth plates from Fgf18+/+; GFP-LC3tg/+ and Fgfl 8+/−; GFP-LC3tg/+ mice at P6. Where indicated, mice were given an IP injection of Tat-Beclin 1 20 mg/kg peptide (once/day for 6 days). The insets show a high magnification of selected areas. Scale bar 10 μm. b, Graph shows quantification of GFP positive dots per cell (mean±s.e.m of n=3 mice/group. *p<0.05, Student's t-test). c, PC2 secretion in mock (untreated) or Spautin1-treated (24h) Rx chondrocytes, incubated for 4 hours at 40° C., and then temperature shifted to 32° C. for 60 min. FGF18 (25 ng/ml) was added where indicated before shifting the temperature to 32° C. The bars represent fractions of secreted collagen expressed as % relative to total collagen (intracellular+secreted)±s.d. of 3 independent experiments. *p<0.05, **p<0.005 ***p<0.0005 Student's t-test). d, Total collagen concentration in femoral and tibia growth plates of Fgf18+/+ and Fgf18+/− mice at P9 treated with Tat-Beclin 1 where indicated (20 mg/kg; daily for 9 days). The concentration of collagen was determined via colorimetric assay using Sirius Red, and values were normalized to total DNA and expressed as % relative to control mice (Fgf18+/+) (mean±s.e.m of 4 mice/group *p<0.05; **p<0.005 ANOVA with post-hoc test). e, Confocal analysis of intracellular Col2a1 in resting chondrocytes of the growth plates in Fgfl 8+/+ and Fgfl 8+/− mice at P6, treated with Tat-Beclin 1 or with vehicle. The insets show a high magnification of selected areas. Red=collagen; Blue=DAPI. Scale bar 10 μm. Graph shows quantification of data (mean±s.e.m of n=3 mice/group. ***p<0.0005 ANOVA with post-hoc).

FIG. 9: a, Comparative TEM images of P0 and P6 wild type growth plate chondrocytes showing increased autophagosomes (AV) biogenesis at P6. Arrows indicate AVs. Bar graphs show number and size of AVs within 5.3 μm field of view (values represent mean±s.e.m. Student's t-test **p<0.005). b, Western blot analysis of LC3I/II of femoral growth plates from mice at indicated ages. Mice were injected with leupeptin (40 mg/kg i.p. 6h before sacrifice) where indicated. β-actin was used as loading control. Bar graphs show quantification of LC3II protein relative to β-actin (mean±s.e.m. *p<0.05 Student's t-test. n=3/group). c, Representative Western blot analysis of Atg7, LC3 and SQSTM1 (p62) proteins in Atg7 f/f, Col2a1-Cre; Atg7 f/f and Prx1-Cre; Atg7 f/f growth plate lysates. Histone 3 (H3) was used as loading control. d, e, f Western blot analysis of Atg7 and LC3 proteins isolated from different tissues isolated from mice with indicated genotypes. GAPDH and β-Actin were used as loading control. Bar graph shows quantification of Atg7 and LC3II proteins in different tissues.

FIG. 10: Alcian blue/Alizarin red skeletal staining of Atg7f/f, Col2a1-Cre; Atg7f/f and Prx1-Cre; Atg7f/f mice at P0 (a), P9 (b), P30 (c) and P120 (d). (Left) Details of femur and tibia magnifications. Graphs show femur and tibia mean lengths from mice with indicated genotypes (values represent mean±s.e.m. Student's t-test *p<0.05, **p<0.005, ***p<0.0005. n=3 mice/genotype). Scale bar 2 mm.

FIG. 11: H/E staining of femural sections of P6 (a) and P9 (b) Atg7 f/f and Prx1-Cre; Atg7 f/f mice showing a reduced femural length starting from P9 in Prx1-Cre; Atg7 f/f compared to control (see black arrowheads). White arrows show normal differentiation of the secondary ossification center in Prx1-Cre; Atg7 f/f compared to control. Scale bar 2 mm. H/E staining of hypertrophic chondrocytes (c), BrDU staining (d), TUNEL assay (e) in P6 Atg7 f/f and Atg7 Ff; Prx1-Cre growth plates (arrows indicate TUNEL positive cells). Graph shows quantification of BrDU index in femural and tibial growth plates from Atg7 f/f and Prx1-Cre; Atg7 f/f mice. (Values represent ±s.e.m. n=3 mice/genotype). Scale bar 100 μm.

FIG. 12: a, Total levels of GAGs in femoral and tibia growth plates of P5 and P9 mice with indicated genotypes. Values were normalized to total DNA and expressed as % relative to P5 control (Atg7f/f) mice. (Values represent mean±s.e.m. ***p<0.0005 ANOVA with post-hoc test. n=5 mice/genotype). b, Extracellular Col2a1 staining in chondroitinase ABC-treated growth plate femoral sections isolated from Atg7 f/f and Prx1-Cre; Atg7 f/f mice. Scale bar 500 μm. c, Confocal analysis of Col2a1, Sec31, VapA (ER), P115 (ER/Golgi), GM130, Giantin (Golgi) and LAMP1 (endsome/lysosome) markers in Prx1-Cre; Atg7f/f growth plate chondrocytes at P6. Insets show high magnification of boxed areas. Scale bar 10 μm. d, Bar graph shows intracellular Col2a1 colocalization (Mander's coefficient) with indicated organelle markers. (At least 2 sections containing 400 cells/section were analyzed for each mouse. N=3 mice).

FIG. 13: a, Western blot analysis of Atg7 and LC3II levels in control (scrambled) and Atg7 siRNA-treated Rx chondrocytes. GAPDH was used as loading control. b, Western blot analysis of LC3II levels in Rx chondrocytes treated with Spautin-1 at indicated concentrations for 24h. β-actin was used as loading control. c, IF staining of LC3 (green) and Col2a1 (red) in chondrocytes treated with BafA1 for 4h. The insets show high magnification and single color channels of the boxed area. Scale bar 10 μm. Bar graph shows area of GFP colocalizing with Col2a1 relative to total GFP area (expressed as %±s.d of at least 500 cells from 2 independent preparations). d,e, Confocal analysis of ATG12 (e), ATG16L (f) (green) and mCherry-PC2 (red) chondrocytes. Blue=DAPI. The insets show a high magnification of selected areas. Scale bar 10 μm. f, IF staining of Col2a1 (blue), HSP47 (red) and GFP-LC3 (green) in Rx chondrocytes, showing that HSP47 does not colocalize with PC2 in AVs. The insets show a high magnification and single color channel of the boxed area. Scale bar 5 μm.

FIG. 14: a a, Immunofluorescence staining of HSP47 chaperone (red) in Atg7^{fl/fl} and Atg7^{fl/fl}; Prx1-Cre chondrocytes, showing altered HSP47 distribution in Prx1-Cre; Atg7^{fl/fl} chondrocytes. The data are representative of 3 independent experiments. Insets show a higher magnification of the boxed area. Scale bar, 10 μm. Blue, DAPI. b, Co-localization of PC2 with HSP47 in growth-plate chondrocytes of mice with indicated genotypes. The data are representative of 3 independent experiments. Scale bar, 20 μm. c, Altered HSP47 and PC2 trafficking in Spautin-1-treated chondrocytes. HSP47 and PC2 immunostaining in control (vehicle) or Spautin-1-treated RCS chondrocytes. Synchronized PC2 secretion was obtained after incubating chondrocytes for 3 h at 40° C. to block PC2 in the ER, and then shifting the temperature to 32° C. (ER block release) for 10 min. The data are representative of 2 independent experiments. Scale bar, 10 μm. d, Proposed model of autophagy function in chondrocytes. Autophagy in chondrocytes prevents PC2 aggregation and maintains ER homeostasis during the process of PC2 secretion. e, Confocal analysis of GFP-LAMP1 (green) and mCherry-PC2 (red) in vehicle- and Spautin-1-treated chondrocytes at the indicated time points (min) after the ER block release. The insets show a high magnification of the selected area. Scale bar, 5 μm. f, Quantification of GFP-LAMP1/mCherry-PC2 co-localization. Values represent mean±s.d. from three independent experiments. N=30. ANOVA, P=4.91×10⁻⁵; Tukey's post-hoc test, ***P<0.0005. g, h, Confocal analysis of RCS chondrocytes treated with tannic acid (0.5% final concentration in the medium) for 1 h, showing that PC2 vesicles (red) at the periphery do not co-localize with LC3 (g) or with LAMP1 (h) (green). The data are representative of 2 independent experiments. Scale bar, 10 μm.

FIG. 15: a, Representative images of high content imaging analysis of primary chondrocytes isolated from GFP-LC3 transgenic mice and treated with vehicle or FGF18 (25 ng /ml for 24 h). BafA1 was used where indicated for 4 h (200 nM). Green spots represent GFP labeled AVs. Scale bar 50 μm. b, Quantification of green vesicles (AVs) in cells treated with the indicated factors for 24h. Vesicles were counted in at least 1000 cells/treatment. Values represent mean values±s.d. of n=3 independent experiments; Statistical analysis was performed using repeated measure ANOVA with TUKEYs post-hoc test. **p<0.005. c, Western blot analysis of primary chondrocytes isolated from wild type mice treated as indicated (FGF18 25 ng/ml, 24 h). Where indicated BafA1 was added (200 nM, 4h). Bar graphs represent mean values±s.d. of n=3 independent experiments. *p<0.05 Student's t-test. d, IF staining of Rx chondrocytes expressing the tandem fluorescent-tagged LC3 (mRFP-EGFP-LC3) protein, showing increased number of autolysosomes and AVs in FGF18 (25 ng/ml for 24 h) treated chondrocytes. As control, cells were treated with BafA1 for 4h (200 nM) to block AV-Lys fusion. Bar graphs show % of red vesicles (autolysosomes) and of total vesicles relative to vehicle (Values represent mean values±s.d. At least 10 cells/experiment were analyzed from 3 independent experiments. *p<0.05 ***p<0.0005. Student's t-test). Scale bar 10 μm. e, Confocal analysis of GFP-LC3 puncta (autophagosomes) in femoral growth plates from P6 GFP-LC3 tg/+; Fgf18+/+ and GFP-LC3 tg/+; Fgfl 8+/− mice. Scale bar 20 μm. Quantification of data (mean±s.e.m of n=5 mice/group. *p<0.05, Student's t-test) f, Western blot analysis of Fgf18+/+ and Fgf18+/− growth plate lysates. Mice were injected with leupeptin (40 mg/kg i.p. 6 h before sacrifice) where indicated. β-actin was used as loading control. Bar graph shows quantification of LC3II protein in vehicle and leupeptin injected mice (Values represent the mean values relative to β-actin±s.e.m. n=3 mice/genotype. *p<0.05, ***p<0.0005 ANOVA with post-hoc test). g, Western blot analysis of P62 protein in three Fgf18+/+ and three Fgfl 8+/− growth plate lysates. β-actin was used as loading control. Bar graph shows quantification of P62 protein (Values represent the mean values±s.e.m. n=3, *p<0.05 Student's t-test).

FIG. 16: a, Representative images of immunofluorescence analysis of LC3 positive vesicles in RCS chondrocytes treated with siRNA for Fgfr1, Fgfr2, Fgfr3 and Fgfr4 and then stimulated with FGF18 for 2 h. BafA1 was added (200 nM, 3 h). Values represent mean values±s.e.m. of n=3 independent experiments (N=40 cells per treatment were analysed). Student's t-test, ***P<0.0005. NS, not significant. Scale bar, 10 μm. b, Immunoprecipitation of FGFR3 or of FGFR4 from RCS chondrocytes stably expressing FGFR3 or FGFR4, respectively, followed by western blotting with phosphotyrosine antibody (pY). Cells were untreated (−) or treated (+) with FGF18 (100 ng ml⁻¹, 20 min). c, Confocal analysis of FGFR3 and FGFR4 in growth-plate chondrocytes isolated from P6 mice. No signal was detected when sections were incubated with secondary antibody alone (Neg. CTR). The data are representative of two independent experiments. Scale bar, 20 μm. d, Western blot analysis of LC3I/II, phospo-JNK1/2, JNK1/2, phospo-ERK1/2, ERK1/2, phospo-P38 MAPK and P38 MAPK in growth plates isolated from three Fgf18^{+/+} and three Fgf18^{+/-} mice at P6. β-Actin was used as a loading control. The bar graph shows quantification of LC3II relative to β-actin and of phosphorylated proteins relative to the corresponding total proteins. Values are mean±s.e.m. from n=3 mice per genotype. Student's t-test, *P<0.05, ***P<0.0005. e, Western blot analysis of three Fgf18^{+/+} and three Fgf18^{+/-} growth-plate lysates showing no differences in the phosphorylation levels of the proteins analysed. Bar graph shows quantification of the ratio of phosphorylated to total protein (values represent mean±s.e.m.; n=3).

FIG. 17: a, Western blot analysis of the phospo-Bcl2 (S70) and of human influenza hemagglutinin (HA) in Rx chondrocytes expressing human Bcl2-HA. Where indicated, chondrocytes were treated with FGF18 (25 ng/ml) for 2h and with JNK inhibitors (50 μM) for 4h. b, Immunoprecipitation assays testing physical interactions between endogenous Beclin 1, Bcl2 and VPS34 in untreated and FGF18-treated Rx chondrocytes. Cells were treated with FGF18 (25 ng/ml) for 2h, and lysates were immunoprecipitated with a Beclin 1-specific antibody or control IgG, followed by probing with antibodies specific for Beclin 1, Bcl2 or VPS34. c, Membrane-associated PI3K assay in situ. Rx chondrocytes were transfected with GFP-2⋅FYVE and then treated with or without FGF18 (25 ng/ml) for 2 h and where indicated, treated with JNK inhibitors (50 μM) for 4h. Graph shows quantitative analysis (mean±s.e.m of number of cells with GFP-2⋅FYVE dots. ***p<0.0005 ANOVA with post-hoc test). Scale bar 10 μm. d, Measure of PI3K activity associated with Beclin 1, expressed as fold change relative to control cells (vehicle treated). Graph shows mean %±s.e.m *p<0.05, Student's t-test. e, Col2a1 (red) and GFP-LC3 (green) confocal analysis of resting chondrocytes in P6 GFP-LC3 tg/+; Fgf18+/− mice showing autophagosomes containing Col2a1 (arrows). The inset shows a high magnification of the boxed areas. Scale bar 10 μm. f, Total collagen concentration in femoral and tibia growth plates of Fgfr4+/+ and Fgfr4−/− mice at P9 treated with TAT-Beclin 1 where indicated. g-h, Femoral lengths of Fgfr4+/+ and Fgfr4−/− mice at P9 (g) and P15 (h) treated with Tat-Beclin 1 where indicated.

FIG. 18: TAT-Beclin 1 expression vector, according to a preferred embodiment of the invention. a, schematic representation of a Tat-Beclin 1 expression cassette for viral delivery of a Tat-Beclin 1 expression vector, according to a preferred embodiment of the invention; b, TAT-beclin overexpression in cell lysates of HEK293 cells transfected with a plasmid encoding for Tat-Beclin 1; c, TAT-beclin overexpression in conditioned media from HEK293 cells 48 hours post-transfection with the plasmid encoding for Tat-Beclin 1. Bec: cell lysate or media from cells transfected with the plasmid encoding for Tat-Beclin 1; neg: cell lysate or media from cells transfected with a negative control plasmid; α-3×flag: Western blot with anti-3×flag antibodies; α-actin: Western blot with anti-actin antibodies, used as loading control. The molecular weight ladder is depicted on the left. d, Cell lysates from HEK293 cells 24 hours post-incubation with Tat-beclin 1 conditioned media. Bec: cells incubated with Tat-Beclin 1conditioned media; neg: cells incubated with media from cells overexpressing a negative control plasmid; α-LC3: Western blot with anti-LC3 antibodies; α-actin: Western blot with anti-actin antibodies, used as loading control. The molecular weight ladder is depicted on the left.

FIG. 19: Altered Autophagy in MPS VII primary chondrocytes. A, Western blot analysis of LAMP1 and LC3II in primary chondrocytes isolated from chondrocostal cartilage of newborn MPS VII and wild-type (wt) mice; B, immunofluorescence of the autophagy receptor p62 in wt and MPS VII primary chondrocytes; C, Double immune labeling of LAMP1 and LC3 in MPS VII and wt primary chondrocytes. Data shown in (B) and (C) are mean+SE of 3 independent experiments.

FIG. 20: Altered mTORC1 signaling in MPS VII primary chondrocytes. a, analysis of p70 S6 Kinase and ULK1 phosphorylation in primary chondrocytes isolated from the rib cage of P5 mice (wt and MPS VII); b, analysis of p70 S6 Kinase and ULK1 phosphorylation in primary chondrocytes in serum or starved for 1h and refed with aminoacids (AA) for 0, 0.3, 2 and 24 hours. c, quantification of analysis shown in (b); d, analysis of p70 S6 Kinase and ULK1 phosphorylation and bar graphs displaying quantification upon serum stimulation alone; e, co-localization of mTORC1 with lysosomes in both starved and nutrient stimulated MPSVII chondrocytes compared to control cells. Data shown in (c) and (e) are mean+SE of 4 and 3 independent experiments, respectively.

FIG. 21: Enhanced mTORC1 signaling in LSD cells. Characterization of Crispr/Cas9 GusbKO RCS clone. A, schematic representation of genetic mutation found in the GusbKO clone: a single base insertion within the first exon causes a frameshift and a premature stop codon within the second exon of the protein. B, the resulted truncated protein lacks enzymatic activity. Bar graph displaying β-glucuronidase enzymatic activity. Enhanced mTORC1 signaling in LSD cells. C-E, Western blot analysis of mTORC1 signaling and bar graphs displaying quantification of relative phosphorylations in GusbKO RCS cells (C), MPS VI (Arsb^{-/-}) mouse primary chondrocytes (D) and MPS I (Mud) differentiated human mesenchimal stem cells (E) and upon a time course of amino acid stimulation. N=3 independent experiments (Student's t-test *p<0.05, **p<0.005).

FIG. 22: Increased mTORC1 association to lysosomes in LSD cells. Primary Arsb^{-/-} (MPS VI) chondrocytes (c-d) were starved for amino acids for 50 min or starved and then re-stimulated with amino acids for the indicated times. Cells were then processed in an immunofluorescence assay to detect mTOR, Lamp-1, costained with DAPI for DNA content, and imaged. The insets show higher magnification and single color channels of the boxed area. Scale bar, 10 μm. Bar graphs display quantitative analysis of co-localization, data are expressed as mean (±s.e.m) of n=3 independent experiments (Student's t-test, * p<0.05, ** p<0.005, *** p<0.0005).

FIG. 23: G, WT and GusbKO RCS cells were pulse labelled for 18h with ³H-Ser and chased for 48h in medium containing vehicle or 100 nM Mg-132. The rate of protein degradation is shown as the fraction of radiolabelled protein remaining over time. Values are expressed as mean (±s.e.m.) of n=3 independent experiments (Student's t-test, * p<0.05, ** p<0.005). H, Luminescent signal resulting from the cleavage of a luminescent Suc-LLVY peptide by the chymotrypsin-like activity of the proteasome was measured in WT and GusbKO RCS cells after 6h treatment with aminoacids. Data are representative of 3 independent experiments and are graphed as relative fluorescence units (RFU) (Student's t-test, * p<0.05). I, Western blot analysis of mTORC1 signaling in WT and GusbKO RCS cells treated with Mg-132 (10 μM) or DMSO (−) for 6h. Arrowheads indicate specific band. J, Bar graphs displaying quantification of relative phosphorylation. Values are expressed as mean (±s.e.m.) of n=3 independent experiments (Student's t-test, * p<0.05, ** p<0.005).

FIG. 24. Autophagy dysfunction in LSD chondrocytes. A, Lamp-1 Immuno-EM from primary cultured chondrocytes isolated from WT (Gusb^{+/+}) and MPS VII (Gusb^{-/-}) mice. Scale bar, 500 nm. B, Western blot analysis of Lamp-1 and LC3 II accumulation in primary cultured chondrocytes with the indicated genotypes. β-Actin was used as a loading control. Blot is representative of 3 independent experiments. C, Immunofluorescence of LC3 in primary chondrocytes isolated from mice with the indicated genotypes. Cells were costained with DAPI for DNA content. Scale bar, 10 μm. Bar graph displays quantification of LC3 vesicles number. Data are means (±s.e.m.) of 3 independent experiments (Student's t-test ***p<0.0005). D, Lamp-1 Immuno-EM from WT and GusbKO RCS cells. Scale bar, 500 nm. Bar graph displays the lysosome size (Student's t-test, *** p<0.0005). E, Western blot analysis of Lamp-1 and LC3 II accumulation in primary cultured chondrocytes with the indicated genotypes. β-Actin was used as a loading control. Blot is representative of 3 independent experiments. F, Immunofluorescence of LC3 in WT and GusbKO RCS cells. Cells were costained with DAPI for DNA content. Scale bar, 10 μm. Bar graph displays quantification of LC3 vesicles number. Data are means (±s.e.m.) of 3 independent experiments (Student's t-test *p<0.05).

FIG. 25. Normal AV biogenesis in MPS VII (Gusb^{-/-}) primary chondrocytes. A, WIPI-2 and LC3 puncta were counted in primary chondrocytes with the indicated genotypes after 24h aminoacids treatment. A statistical analysis was performed using an unpaired Student's t-test, *** p<0.0005. B, Western blot analysis of LC3II accumulation in presence of the lysosomal inhibitor Bafilomycin A1 (200 nm) for the indicated time points. The rate of autophagosome formation was calculated using the ratio of accumulated LC3 II between 3h and 1h of treatment. N=3 independent experiments. C, Western blot analysis showing phosphorylation of ULK1 by AMPK at S555 and S317. D, Immunofluorescence analysis of TFEB and TFE3 nuclear localization in primary chondrocytes with the indicated genotype after 50 minutes of amino acid starvation (STV) and upon 24h of amino acid stimulation (fed). Cells were costained with DAPI to define nuclear region. E, Bar graphs displaying quantification of the percentage of cells positive for nuclear translocation. The data are representative of 3 independent experiments, n≥90 cells were analyzed for each time point. Scale bar, 10 μm (Student's t-test, *** p<0.0005).

FIG. 26. Impaired Av-Lys fusion in LSD cells. A, Immunofluorescence of Lamp-1, p62 and LC3 in primary chondrocytes isolated from mice with the indicated genotypes. The insets show higher magnification, single color channels, Lamp-1-p62 and Lamp-1-LC3 co-localization of the boxed area. Scale bar, 10 μm. B, Quantification of Lamp-1 co-localization with LC3 and p62. Data are Mander's coefficient means (±s.e.m.)(ImageJ plug-in) of 3 independent experiments (Student's t-test **p<0.005, *p<0.05). C, Western blot analysis of SQSTM1/p62 accumulation. β-Actin was used as a loading control. Blot is representative of 3 independent experiments. D, Immunofluorescence of Lamp-1, p62 and LC3 in WT and GusbKO RCS cells. Scale bar, 10 μm. E, Quantification of Lamp-1 co-localization with LC3 and p62. Data are Mander's coefficient means (±s.e.m.) of 3 independent experiments (Student's t-test **p<0.005, *p<0.05). F, Western blot analysis of SQSTM1/p62 accumulation. β-Actin was used as a loading control. Blot is representative of 3 independent experiments. G, RFP-GFP-LC3 was transiently expressed in RCS cells with the indicated genotypes. LC3 was monitored by fluorescence microscope two days post transfection. Scale bar, 10 μm. Bar graph displays quantitative analysis of RFP-only puncta per cell (Student's t-test **p<0.005).

FIG. 27. Altered PC2 trafficking in MPS VII(Gusb^{-/-}) chondrocytes. A, Golgin and PC2 immunostaining in WT (Gusb^{+/+}) or MPS VII (Gusb^{-/-}) chondrocytes. Synchronized PC2 secretion was obtained after incubating chondrocytes for 3 h at 40° C. to block PC2 in the ER, and then shifting the temperature to 32° C. (ER block release) for 15 min. B, Bar graph displaying quantification of Golgin-PC2 co-localization. The data are Mander's Coefficient means (±s.e.m.) representative of 2 independent experiments, n≥90 cells were analyzed for each experiment and time point. Scale bar, 10 μm (Student's t-test, *** p<0.0005).

FIG. 28: Pharmacological inhibition of mTORC1 restores autophagy flux in MPS VII chondrocytes. α-b, biochemical analysis in primary chondrocytes treated with Torin1 (1 μM) for 24 hours (a) and quantification (b). Data shown in (b) are mean+SE of 3 independent experiments.

FIG. 29: Genetic limitation of mTORC1 in MPS VII chondrocytes rescues both mTORC1 altered signaling and autophagy flux. a, LC3II, phosphor-ULK1 (P-ULK1) and phosphor-p70 S6K (P-p70S6K) levels in primary chondrocytes isolated from MPS VII and Raptor (RPT) mice; b, p62 puncta in primary chondrocytes isolated from MPS VII and Raptor (RPT) mice; c, autophagosome-lysosome fusion in primary chondrocytes isolated from MPS VII and Raptor (RPT) mice. Samples loaded in (a) represent 3 independent cellular preparation for each genotype.

FIG. 30: A, Western blot analysis of mTORC1 signaling in primary cultured chondrocytes isolated from Gusb^{-/-} and Gusb^{-/-}; Rpt^{+/-} mice upon a time course of amino acid stimulation. B, Quantification of normalized phosphorylation relative to Gusb^{-/-} (ANOVA, P=0.009; *p<0.05). C, Western blot analysis of LC3I/II, p62 and Raptor levels in chondrocytes isolated from mice with the indicated genotypes. β-Actin was used as a loading control. Blot is representative of 3 independent experiments. D, quantification of protein amount normalized to β-actin and relative to Gusb^{-/-}. Analysis of variance (ANOVA) P=0.0064; Tukey's post-hoc test, ** p<0.005, * p<0.05, ns: not significant. E, Immunofluorescence of Lamp-1, p62 and LC3 in primary chondrocytes isolated from mice with the indicated genotypes. The insets show higher magnification, single color channels, Lamp-1-p62 and Lamp-1-LC3 co-localization of the boxed area. Scale bar, 10 μm. F, quantification of Lamp-1 co-localization with LC3 and p62. Data are Mander's coefficient means (±s.e.m.)(ImageJ plug-in). ANOVA Lamp-1-LC3 P=7.39E−06, Lamp1-p62 P=0.008; Tukey's post-hoc test, *p<0.05; *** p<0.0005. G, quantification of p62 puncta of cells shown in D (ANOVA P=4.67E−05; Tukey's post-hoc test *** p<0.005, *p<0.05). H, RFP-GFP-LC3 was transiently expressed in primary chondrocytes with the indicated genotypes. LC3 was monitored by fluorescence microscope two days post-transfection and after 24h amino acid treatment. Scale bar, 10 μm. I-L, Quantitative analysis of GFP puncta (I) and RFP-only puncta (L). Mean value of 3 independent experiments is shown as a horizontal bar (ANOVA, GFP puncta P=0.002, RFP puncta P=1.63E−06; Tukey's post-hoc test, *** p<0.0005, ** p<0.005).

FIG. 31: Normal AV biogenesis in Gusb^{-/-}; Rpt^{+/-} primary chondrocytes. Western blot analysis of LC3II accumulation in presence of the lysosomal inhibitor Bafilomycin A1 (200 nm) for the indicated time points. The rate of autophagosome formation was calculated using the ratio of accumulated LC3 II between 3h and 1h of treatment.

FIG. 32: mTORC1 inhibits AV-Lys fusion in MPS via UVRAG. A, immunoprecipitation assay testing the increase of UVRAG phosphorylation in RCS GusbKO relative to RCS WT cells. The increase is blunted in the presence of Torin-1 (1 μM; 6h). B, immunoprecipitation assay testing physical interactions between endogenous UVRAG, Rubicon and Beclin-1 in RCS WT and GusbKO RCS chondrocytes after 6h amino acid treatment. Cell lysates were immunoprecipitated with an UVRAG-specific antibody followed by probing with antibodies specific for P-UVRAG (S498), UVRAG, Rubicon or Beclin-1. C, myc-UVRAG was transiently expressed in Gusb^{-/-} primary chondrocytes. Myc expression, LC3 and P62 were monitored by fluorescence microscope two days post-transfection and after 24h amino acid treatment. Scale bar, 10 μm. Quantitative analysis of P62 and LC3 puncta. Mean value is shown as a horizontal bar. (Student's t-test, *** p<0.0005, n≥20). D, Western blot analysis of LC3I/II and p62 in WT and GusbKO RCS treated with Tat-Beclin-1 and inactive Tat-Beclin-1 (Tat-Beclin-1-m). β-Actin was used as a loading control. Blot is representative of 3 independent experiments. E, quantification of protein amount normalized to β-actin and relative to RCS WT (ANOVA, P62 P<0.0001, Tukey's post-hoc test, ***p<0.0005, **p<0.005, *p<0.05). F, Immunofluorescence of Lamp-1 and LC3 in GusbKO cells treated with Tat-Beclin-1 peptide (10 μM; 2h). Scale bar, 10 μm. Quantification of Lamp-1-LC3 co-localization is shown as mean (±s.e.m.) of Mander's Coefficients resulting from three independent experiments (Student's t-test, *p<0.05).

FIG. 33: Limitation of mTORC1 signaling for the treatment of bone growth retardation in MPS VII mice. a, femur and tibia sections of wt, MPS VII and RPT mice at P15; b, femur and tibia length analysis at P15; c, Representative images of P15 femoral growth plates sections from wt (Gusb^{+/+}), MPS VII (Gusb^{-/-}) and RPT (Gusb^{-/-}; Rpt^{+/-})mice at P15. Panels i-iii, staining with hematoxylin & eosin (H&E) shows the regions chosen for the analysis. Panels iv-xv, immunostaining with P-S6 (iv-vi), p62 (vii-ix), Col1 X (x-xii) and Col1 II (xiii-xv), BrdU staining (lower panel). Nuclei were counterstained with hematoxylin or DAPI (p62). n=5 mice per genotype. Scale bar (100 μm). a Haematoxylin/Eosin (H&E) and Collagen type X immunostaining of femur and tibia sections of wt, MPS VII and RPT mice at P15; d, quantification of proliferative and hypertrophic zones, % of BrdU positive cells and amount of collagen (% of WT) in the growth plate homogenates. For growth plate length measure and quantification of BrdU labeling, sections from at least six animals of each genotype were analyzed.; e, femur and tibia length analysis at P30.

FIG. 34: Lysosomal storage in chondrocytes. EM from growth plates isolated from P6 WT⁺, MPS VII and RPT mice. Scale bar, 500 nm.

### DETAILED DESCRIPTION OF THE INVENTION

The present invention is directed to a molecule capable of activating Beclin 1/Vps34 complex in a cell for use in the treatment and/or prevention of a bone growth disorder; more preferably, said cell is a chondrocyte; most preferably said cell is a mammalian cell.

An activator of Beclin 1/Vps34 is a molecule that favors vps34 PI3K Beclin 1-dependent activity. Activation of Beclin 1/Vps34 complex directly leads to the increase of PI3P levels. In other words, activators of Beclin 1/Vps34 complex stimulate Beclin 1-dependent lipid kinase activity of Vps34. Vps34 kinase activity upregulates the phosphatidylinositol 3-phosphates (PI3P) at the phagophore. Activators of Beclin 1/Vps34 complex increase PI3P production in a cell.

Activation of Beclin 1/Vps34 complex can thus be assessed by any assay for measuring the levels of PI3P at the phagophore. An exemplary assay is the membrane-associated PI3 Kinase (PI3K) assay in situ, as described herein. FYVE is a domain that binds with great specificity to PI3P. 2×FYVE-EGFP transfected in a cell localizes to early endosomes in a PI3K activity-dependent fashion (Pattini et al, 2001). In cells transfected with GFP-2⋅FYVE and then treated with a potential activator of Beclin 1/Vps34 complex, EGFP puncta increase compared to control cells (vehicle treated). Further methods include the analysis of PI3K activity in Beclin 1 immunoprecipitates, using commercial PI3K ELISA kits, according to the manufacturer's instructions.

An inhibitor of mTORC1 is a molecule capable of prevent either phosphorylation of proteins substrates or autophosphorylation of mTOR. In particular, an activator of Beclin 1/Vps34 complex, which is an inhibitor of mTORC1, according to a preferred embodiment of the invention, is a molecule capable of reducing ULK1 phosphorylation by mTORC1.

ULK1 phosphorylation reduction can be assessed for example as described herein by measuring the relative levels of Phospo-ULK1 proteins.

A small molecule is a low molecular weight (<900 daltons) organic compound, with a size on the order of 10⁹ m. A small molecule binds to a specific biological target—such as a specific protein or nucleic acid—and acts as an effector, altering the activity or function of the target.

Preferred inhibitors of mTORC1 comprise: Rapamycin (CAS No. 53123-88-9), KU0063794 (CAS No. 938440-64-3), WYE354 (CAS No. 1062169-56-5), Deforolimus (CAS No. 572924-54-0), TORN 1 (CAS No. 1222998-36-8), TORN 2 (CAS No. 1223001-51-1), Temsirolimus (CAS No. 162635-04-3), Everolimus (CAS No. 159351-69-6), sirolimus (CAS No. 53123-88-9), NVP-BEZ235 (CAS No. 915019-65-7), PI103 (CAS No. 371935-74-9).

BH3 mimetics are small molecules capable of mimicking BH3-only proteins of the BCL-2 family, i.e. having only the BCL-2 homology domain BH3.

Bcl-2 homology (BH) domains: BH3 domain in Beclin 1 is similar to that required for the binding of proapoptotic proteins to antiapoptotic Bcl-2 homologs. Typically, a BH3 domain is defined as a four-turn amphipatic α-helix, bearing the sequence motif: Hy-X-X-X-Hy-K/R-X-X-Sm-D/E-X-Hy, in which Hy are hydrophobic residues and Sm represents small residues, typically glycine. Proapoptotic Bcl-2 proteins are grouped into two categories: (1) the multidomain proapoptotic proteins that contain three BH domains, BH4, BH3 and BH1; and (2) the BH3-only proapoptotic proteins that contain only the BH3 domain. In both these groups, the BH3 domain is required for interaction with antiapoptotic Bcl-2 proteins. The BH3-only proteins are thus a subset of the Bcl-2 family of proteins, containing only a single BH3-domain. The BH3-only family members are Bim, Bid, BAD and others. Various apoptotic stimuli induce expression and/or activation of specific BH3-only family members, which translocate to the mitochondria and initiate Bax/Bak-dependent apoptosis. BH3-mimetics promote dissociation of Beclin-1 from BclXL thus making Beclin-1 able to enter into the initiating complex comprising Vps34 and Vps15. Preferred BH3 mimetics for use according to the present invention comprise: ABT-737, ABT-263/navitoclax, Obatoclax, Gossypol, AT-101, Apogossypol, Apogossypolone/ApoG2, BI-97C1/sabutoclax, TW37, S1, 072RB, SAHB-A, BIMS2A, Mc1-1 SAHB (Billard, 2013).

In the present invention a Beclin 1 peptide refers to accession number NP_003757 (SEQ ID No. 45)

MEGSKTSNNSTMQVSFVCQRCSQPLKLDTSFKILDRVTIQELTAPLLTTAQ
AKPGETQEEETNSGEEPFIETPRQDGVSRRFIPPARMMSTESANSFTLIGE
ASDGGTMENLSRRLKVTGDLFDIMSGQTDVDHPLCEECTDTLLDQLDTQLN
VTENECQNYKRCLEILEQMNEDDSEQLQMELKELALEEERLIQELEDVEKN
RKIVAENLEKVQAEAERLDQEEAQYQREYSEFKRQQLELDDELKSVENQMR
YAQTQLDKLKKTNVFNATFHIWHSGQFGTINNFRLGRLPSVPVEWNEINAA
WGQTVLLLHALANKMGLKFQRYRLVPYGNHSYLESLTDKSKELPLYCSGGL
RFFWDNKFDHAMVAFLDCVQQFKEEVEKGETRFCLPYRMDVEKGKIEDTGG
SGGSYSIKTQFNSEEQWTKALKFMLTNLKWGLAWVSSQFYNK

or a peptide encoded by an ortholog gene thereof.

In the present invention a Beclin 1 peptide fragment is a peptide comprising a subsequence of a Beclin 1 peptide; a Beclin 1 peptide fragment is thus a peptide shorter than the Beclin 1 peptide whose sequence is reported above; preferably said fragment or subsequence comprises residues 270-278 of a Beclin 1 peptide, more preferably it comprises residues 269-283 of a Beclin 1 peptide. Preferably said Beclin 1 fragment comprises at least 3 amino acid residues, preferably at least 5, at least 6, at least 8, at least 10, at least 15 or at least 20 amino acid residues. Preferably, said Beclin 1 peptide fragment has at least 65%, at least 70%, at least 80%, at least 90%, at least 95% identity with the Beclin 1 peptide. Said Beclin 1 peptide fragment maintains the biological activity of Beclin 1, i.e. activation of the Beclin 1/Vps34 complex, so that said fragment may treat or prevent a bone growth disorder.

In the present invention a Beclin 1 peptide derivative is a peptide comprising a Beclin 1 peptide or a Beclin 1 peptide fragment or the retro-inverso peptide thereof, and comprising alternative structures and/or formulations of said Beclin 1 peptide or of said Beclin 1 peptide fragment or of said retro-inverso peptide thereof.

For instance, said Beclin 1 derivative peptide may comprise at least one heterologous moiety (i.e. a moiety deriving from a different species), and/or may be chemically modified. The derivative maintains the biological activity of Beclin 1, i.e. activation of the Beclin 1/Vps 34 complex, so that said derivative may treat or prevent a bone growth disorder. In an exemplary non-limiting embodiment, a Beclin 1 peptide derivative is a peptide comprising residues 270-278 of a Beclin 1 peptide, optionally flanked by no more than twelve naturally-flanking Beclin 1 residues, wherein up to six residues may be substituted, and linked to a heterologous moiety. According to an exemplary non-limiting embodiment, a peptide derivative is a peptide comprising the Beclin 1 peptide or a fragment thereof or a retro-inverso peptide thereof and having amino acid residue substitution(s). Preferably said derivative comprises from 1 to 6 amino acid residue substitution(s).

Retro-inverso peptides are linear peptides whose amino acid sequence is reversed and the α-center chirality of the amino acid subunits is inverted as well. Usually, these types of peptides are designed by including D-amino acids in the reverse sequence to help maintain side chain topology similar to that of the original L-amino acid peptide and make them more resistant to proteolytic degradation. Other reported synonyms for these peptides in the scientific literature are: Retro-Inverso Peptides, All-D-Retro Peptides, Retro-Enantio Peptides, Retro-Inverso Analogs, Retro-Inverso Analogues, Retro-Inverso Derivatives, and Retro-Inverso Isomers. D-amino acids represent conformational mirror images of natural L-amino acids occurring in natural proteins present in biological systems. Peptides that contain D-amino acids have advantages over peptides that just contain L-amino acids. In general, these types of peptides are less susceptible to proteolytic degradation and have a longer effective time when used as pharmaceuticals. Furthermore, the insertion of D-amino acids in selected sequence regions as sequence blocks containing only D-amino acids or in-between L-amino acids allows the design of peptide based drugs that are bioactive and possess increased bioavailability in addition to being resistant to proteolysis. Furthermore, if properly designed, retro-inverso peptides can have binding characteristics similar to L-peptides. Retro-inverso-peptides are attractive alternatives to L-peptides used as pharmaceuticals. These type of peptides have been reported to elicit lower immunogenic responses compared to L-peptides. In the present invention a retro-inverso sequence is thus a reversed sequence wherein the α-center chirality of the amino acid subunits is inverted as well. Preferably, the retro-inverso peptide comprises all D-amino acids. As an example: the retro-inverso peptide of a peptide of sequence VFNATFHIWHSGQFG (SEQ ID No. 13) would be a peptide of sequence GFQGSHWIHFTANFV (SEQ ID No. 46). The availability of modern chemical synthesis methods allows the routine synthesis of these types of peptides.

Preferably, the molecule of the invention is for use in the treatment and/or prevention of a bone growth disorders. Exemplary bone growth disorders include achondroplasia, hypochondroplasia, MPS I, MPS II, MPS IV, MPS VI, MPS VII, MPS IX, Gaucher disease type 3, Gaucher disease type 1, a glycoproteinoses, pycnodysostosis. Further bone growth disorders include bone disorders with collagen involvement such as the group of spondyloepiphyseal dysplasias.

Beclin 1/Vps34 complex is a protein complex comprising Beclin 1 protein (NP_003757) and Vps34 protein (NP_001294949; NP_002638). The activation of said complex is capable of inducing autophagic response in a cell; as an example the activation of said complex can induce the first step of autophagosome formation, the nucleation of the phagophore at the endoplasmic reticulum (autophagic vesicle nucleation). Further components of the active Beclin-1/Vps34 complex include Vps15 protein (NP_055417). Optionally the active Beclin-1/Vps34 complex includes Atg14L (NP_055739); optionally the active Beclin-1/Vps34 complex includes UVRAG protein (NP_003360); optionally the active Beclin-1/Vps34 complex includes Ambral protein (NP_060219). Preferably, the active Beclin 1/Vps34 complex does not include Rubicon protein (NP_001139114), which has been shown to negatively regulate the Beclin 1/Vps34 complex.

Preferably, the molecule of the invention for use in the treatment of a bone growth disorder, capable of activating a Beclin 1/Vps34 complex, induces autophagy and/or promotes endocytic trafficking. Therefore, preferably, a molecule capable of activating Beclin 1/Vps34 complex in a cell is a molecule capable of inducing autophagy in a cell, more preferably a molecule capable of inducing formation of autophagosomes and of autophagosomes-lysosome fusion in a cell.

In order to assess autophagic cellular response, autophagosome biogenesis (WIPI2 and Atg16 positive dots), maturation (LC3-LAMP1 positive vesicles) and substrate degradation (long lived protein and p62 degradation) rates can be measured.

In a cell or tissue, activation of Beclin 1-Vps 34 complex can be detected directly, indirectly or inferentially by conventional assays, such as disclosed and/or exemplified herein. Activation of Beclin 1/Vps34 complex in a cell can be assessed by several methods known on the art. In particular, the activation of Beclin 1/Vps34 can be assessed and measured by measuring the PI3P production in in a cell, in a tissue and/or in the growth plates from treated and untreated subjects. Further methods include the quantification by western blot and immunofluorescence analyses of the levels of p62, LAMP1 and LC3II proteins in a cell, in a tissue and/or in the growth plates from treated and untreated subjects.

An activator according to the invention for use in the treatment and/or prevention of a bone growth disorder can thus be identified by quantification by western blot and/or immunofluorescence analyses of the levels of p62, LAMP1 and LC3II proteins.

In order to quantify the fraction of lysosome and autophagosome vesicles at different stages of maturation transmission electron microscopy on growth plate and cortical bone sections of treated and untreated subjects can be performed.

mTORC1 activity can be assessed by measuring the relative levels of phospho-p70 S6K and of Phospo-ULK1 proteins in growth plate and bone extracts. Also, the intracellular localization of TFEB and of TFE3 (nuclear vs cytosolic) by immunohistochemistry can be monitored, as well as the expression levels of autophagy and lysosomal genes by qPCR. Inhibition of mTORC leads to activation of Beclin 1/Vps34 complex and consequently to induction of cellular autophagy/endocytic trafficking. Inhibition of mTORC can thus be measured by the assays herein described aimed at measuring activation of Beclin 1/Vps34 complex.

Preferably, the molecule of the invention for use in the treatment of a bone growth disorder is selected from the group comprising: a Beclin 1 peptide fragment, a Beclin 1 derivative peptide, an mTORC1 inhibitor or a BH3 mimetic.

According to a preferred embodiment, the molecule of the invention for use in the treatment of a bone growth disorder is a Beclin 1 peptide fragment comprising residues 270-278 of Beclin 1 protein sequence or a fragment comprising residues 269-283 of Beclin 1 protein sequence, or retro-inverso sequence thereof.

According to a preferred embodiment, the molecule of the invention for use in the treatment of a bone growth disorder is a Beclin 1 derivative peptide; more preferably said Beclin 1 derivative peptide comprises: (a) residues 269-283 of Beclin 1 protein sequence immediately flanked on each terminus by no more than twelve naturally-flanking Beclin 1 residues, wherein up to six of said residues 269-283 may be substituted, and (b) a first heterologous moiety.

According to preferred embodiments, the molecule of the invention for use in the treatment of a bone growth disorder can consist in a Beclin 1 derivative peptide, said Beclin 1 derivative peptide comprising: (a) residues 269-283 of Beclin 1 protein sequence (VFNATFHIWHSGQFG; SEQ ID NO:13) immediately flanked on each terminus by no more than twelve naturally-flanking Beclin 1 residues, wherein up to six of said residues 269-283 may be substituted, and (b) a first heterologous moiety, such as wherein:

the peptide is N-terminally flanked with T-N and C-terminally flanked by T;

the peptide comprises at least one of F270, F274 and W277;

the peptide comprises at least one substitution, particularly of H275E, S279D or Q281E;

the peptide is N-terminally joined to the first moiety, and C-terminally joined to a second heterologous moiety;

the peptide is joined to the first moiety through a linker or spacer; preferably the linker or spacer is a a diglycine linker. the first moiety comprises a transduction domain, including: protein-derived (e.g. Tat (SEQ ID NO: 44), smac (Accession number GenBank: AAF87716.1), pen (ALC39141.1), pVEC, bPrPp (ALS90899.1), PIs1 (A1RQH3.1), VP22 (ANR01123.1), M918 (EQB90450.1), pep-3 (AAA34852.1)), chimeric (e.g. TP (CAE48349.1), TP10 (CAI48908.1), MPGA (XP 637125.1)), and synthetic (e.g. MAP (CAJ99007.1), Pep-1 (AAQ01688.1), oligo-Arg cell-penetrating peptides;

the first moiety comprises a homing peptide, such as RGD-4C, NGR (Q9N0E3.1), CREKA, LyP-1 (XP_009259791.1), F3 (ABA26022.1), SMS (AAA97285.1), IF7 (NP_035129.1) or H2009.1 (AIG45257.1);

the first moiety comprises a stabilizing agent, such as a PEG, oligo-N-methoxyethyl glycine (NMEG), albumin, an albumin-binding protein, or an immunoglobulin Fc domain;

the peptide comprises one or more D-amino acids, L-P-homo amino acids, D-β-homo amino acids, or N-methylated amino acids;

the peptide is cyclized;

the peptide is acetylated, acylated, formylated, amidated, phosphorylated, sulfated or glycosylated;

the peptide comprises an N-terminal acetyl, formyl, myristoyl, palmitoyl, carboxyl or 2-furosyl group, and/or a C-terminal hydroxyl, amide, ester or thioester group;

the peptide comprises an affinity tag or detectable label; and/or the peptide is N-terminally joined to the first moiety, and C-terminally joined to a second heterologous moiety comprising a detectable label, such as a fluorescent label. Labels and tags are known in the art.

Particular embodiments include all combinations and sub-combinations of particular embodiments, such as wherein: the peptide is N-terminally flanked with T-N and C-terminally flanked by T, the first moiety is a tat protein transduction domain linked to the peptide through a diglicine linker; and the peptide is N-terminally flanked with T-N and C-terminally flanked by T, the first moiety is a tetrameric integrin a(v)P(6)-binding peptide known as H2009.1, linked to the peptide through a maleimide-PEG(3) linker.

In a preferred aspect of the invention the molecule is a Beclin 1 derivative peptide comprising: (a) Beclin 1 residues 269-283 (SEQ ID No. 13) immediately flanked on each terminus by no more than 12 (or 6, 3, 2, 1 or 0) naturally-flanking Beclin 1 residues, wherein up to six (or 3, 2, 1 or 0) of said residues 269-283 may be substituted, and (b) a first heterologous moiety. In some embodiments the peptide may be N-terminally flanked with TN and C-terminally flanked by T (TNVFNATFHIWHSGQFGT; SEQ ID NO:14). In some embodiments the peptide comprises at least one (or two or three) of substitutions: H275E, S279D and Q281E (e.g. VFNATFEIWHDGEFG; SEQ ID NO:15).

In other embodiments the peptide comprises at least one (or two or three) of F270, F274 and W277.

Peptides activity according to preferred embodiments of the invention are also tolerant to backbone modification and replacement, side-chain modifications, and N- and C-terminal modifications, all conventional in the art of peptide chemistry.

Chemical modifications of the peptides bonds may be used to provide increased metabolic stability against enzyme-mediated hydrolysis; for example, peptide bond replacements (peptide surrogates), such as trifluoroethylamines, can provide metabolically more stable and biologically active peptidomimetics.

Modifications to constrain the peptides backbone include, for example, cyclic peptides/peptidomimetics which can exhibit enhanced metabolic stability against exopeptidases due to protected C- and N-terminal ends. Suitable techniques for cyclization include Cys-Cys disulfide bridges, peptide macrolactam, peptide thioether, parallel and anti-parallel cyclic dimers, etc.

Other suitable modifications include acetylation, acylation (e.g. lipopeptides), formylation, amidation, phosphorylation (on Ser, Thr and/or Tyr), etc. which can be used to improve peptide bioavailability and/or activity, glycosylation, sulfonation, incorporation of chelators (e.g. DOTA, DPTA), etc. PEGylation can be used to increase peptide solubility, bioavailability, in vivo stability and/or decrease immunogenicity, and includes a variety of different PEGs: HiPEG, branched and forked PEGs, releasable PEGs; heterobifunctional PEG (with endgroup N-Hydroxysuccinimide (NHS) esters, maleimide, vinyl sulfone, pyridyl disulfide, amines, and carboxylic acids), etc.

Suitable terminal modifications include N-terminal acetyl, formyl, myristoyl, palmitoyl, carboxyl and 2-furosyl, and C-terminal hydroxyl, amide, ester and thioester groups, which can make the peptide more closely mimic the charge state in the native protein, and/or make it more stable to degradation from exopeptidases.

According to preferred embodiments, the peptides may also contain atypical or unnatural amino acids, including D-amino acids, L-P-homo amino acids, \ddot{ν}-β-homo amino acids, N-methylated amino acids, etc.

In a particular embodiment, the peptide is N-terminally joined to a first moiety, heterologous to (not naturally flanking) the Beclin 1 peptide, typically one that promotes therapeutic stability or delivery, and C-terminally joined to a second moiety, preferably also heterologous to the Beclin 1 peptide. A wide variety of such moieties may be employed, such as affinity tags, transduction domains, homing or targeting moieties, labels, or other functional groups, such as to improve bioavailability and/or activity, and/or provide additional properties.

One useful class of such moieties include transduction domains which facilitate cellular penetrance or uptake, such as protein-derived (e.g. tat, smac, pen, pVEC, bPrPp, PIs1, VP22, M918, pep-3); chimeric (e.g. TP, TP10, MPGA) or synthetic (e.g. MAP, Pep-1, Oligo Arg) cell-penetrating peptides; see, e.g. “Peptides as Drugs: Discovery and Development”, Ed. Bernd Groner, 2009 WILEY-VCH Verlag GmbH & Co, KGaA, Weinheim, esp. Chap 7: “The Internalization Mechanisms and Bioactivity of the Cell-Penetrating Peptides”, Mats Hansen, Elo Eriste, and Ulo Langel, pp. 125-144.

Another class are homing biomolecules, such as RGD-4C, NGR, CREKA, LyP-1, F3, SMS (SMSIARL, SEQ ID No. 47), IF7, and H2009.1 (Li et al. Bioorg Med Chem. 2011 Sep. 15; 19(18):5480-9), particularly cancer cell homing or targeting biomolecules, wherein suitable examples are known in the art, e.g. Homing peptides as targeted delivery vehicles Pirjo Laakkonen and Kirsi Vuorinen, Integr. Biol., 2010, 2, 326-337; Mapping of Vascular ZIP Codes by Phage Display, Teesalu T, Sugahara K N, Ruoslahti E., Methods Enzymol. 2012; 503:35-56.

Other useful classes of such moieties include stabilizing agents, such as PEG, oligo-N-methoxyethylglycine (NMEG), albumin, an albumin-binding protein, or an immunoglobulin Fc domain; affinity tags, such as immuno-tags, biotin, lectins, chelators, etc.; labels, such as optical tags (e.g. Au particles, nanodots), chelated lanthanides, fluorescent dyes (e.g. FITC, FAM, rhodamines), FRET acceptor/donors, etc.

The moieties, tags and functional groups may be coupled to the peptide through linkers or spacers known in the art, such as polyglycine, c-aminocaproic, etc.

The peptide can also be presented as latent or activatable forms, such as a prodrug, wherein the active peptide is metabolically liberated; for example, release of the linear peptide from cyclic prodrugs prepared with an acyloxyalkoxy promoiety (prodrug 1) or a 3-(2′-hydroxy-4′,6′-dimethylphenyl)-3,3-dimethyl propionic acid promoiety (prodrug 2) of the peptide).

According to a preferred embodiment, said peptide comprises one or more D-amino acids, L-β-homo amino acids, O-β-homo amino acids, or N-methylated amino acids.

According to a preferred embodiment, said compound comprises an affinity tag or detectable label.

According to a preferred embodiment, said peptide is N-terminally joined to the first moiety, and C-terminally joined to a second heterologous moiety comprising a fluorescent label.

According to a preferred embodiment, said peptide is N-terminally flanked with T-N and C-terminally flanked by T, the first moiety is a tat protein transduction domain linked to the peptide through a diglycine linker.

According to a preferred embodiment, said peptide is N-terminally flanked with T-N and C-terminally flanked by T, the first moiety is a tetrameric integrin a(v)P(6)-binding peptide known as H2009.1, linked to the peptide through a maleimide-PEG(3) linker.

In a further aspect of the invention, the molecule capable of activating Beclin-1/Vps34 complex for use in the treatment and/or prevention of a bone growth disorder is a Beclin 1 derivative peptide comprising Beclin 1 residues 270-278 (FNATFHIWH; SEQ ID NO: 16), or the D-retro-inverso sequence thereof, immediately N- and C-terminally flanked by moieties R1 and R2, respectively, wherein up to six of said residues may be substituted, R1 and R2 do not naturally flank the Beclin 1 residues, and F270 and F274 are optionally substituted and optionally linked.

In particular embodiments of the invention said peptide's sequence is unsubstituted or up to six of said residues may be substituted, and the two F residues are F1 and F2 and are optionally substituted and optionally linked, or said compound has D-retro-inverso sequence of said peptide; optionally wherein:
R1 is a heterologous moiety that promotes therapeutic stability or delivery of the compound;
R1 comprises a transduction domain, a homing peptide, or a serum stabilizing agent;
R1 is a tat protein transduction domain linked to the peptide through a diglycine linker, particularly a diglycine-T-N linker;
R2 is carboxyl or R2 comprises an affinity tag or detectable label, particularly a fluorescent label;
F270 and F274 are substituted and linked;
F270 and F274 are substituted with cross-linkable moieties and/or linked, and each optionally comprises an additional α-carbon substitution selected from substituted, optionally hetero-lower alkyl, particularly optionally substituted, optionally hetero-methyl, ethyl, propyl and butyl; or F270 and F274 are substituted with homocysteines connected through a disulfide bridge to generate a ring and tail cyclic peptide;
the side chains of F270 and F274 are replaced by a linker:
—(CH2)nONHCOX(CH2)m-, wherein X is CH2, NH or O, and m and n are integers 1-4, forming a lactam peptide; CH2OCH2CHCHCH2OCH2-, forming an ether peptide; or (CH2)nCHCH(CH2)m-, forming a stapled peptide;
1 to 6 residues are alanine substituted; or the peptide comprises at least one of substitutions: H275E and S279D; or the peptide comprises one or more D-amino acids, E-β-homo amino acids, O-β-homo amino acids, or N-methylated amino acids; or the peptide comprises the D-retro-inverso sequence, preferably RRQRRKKKRGYGG DHWIEFTANFV (SEQ ID NO: 12);
wherein the peptide is acetylated, acylated, formylated, amidated, phosphorylated, sulfated or glycosylated;
comprising an N-terminal acetyl, formyl, myristoyl, palmitoyl, carboxyl or 2-furosyl group, and/or a C-terminal hydroxyl, amide, ester or thioester group; and/or
wherein the peptide is cyclized.

The invention includes all combinations of the recited particular embodiments above, as if each combination had been laboriously separately recited.

Peptides and compound activity are tolerant to a variety of additional moieties, flanking residues, and substitutions within the defined boundaries. Peptide and compound activity are also tolerant to backbone modification and replacement, side-chain modifications, and N- and C-terminal modifications, all conventional in the art of peptide chemistry.

Chemical modifications of the peptides bonds may be used to provide increased metabolic stability against enzyme-mediated hydrolysis; for example, peptide bond replacements (peptide surrogates), such as trifluoroethylamines, can provide metabolically more stable and biologically active peptidomimetics.

Modifications to constrain the peptides backbone include, for example, cyclic peptides/peptidomimetics which can exhibit enhanced metabolic stability against exopeptidases due to protected C- and N-terminal ends. Suitable techniques for cyclization include Cys-Cys disulfide bridges, peptide macrolactam, peptide thioether, parallel and anti-parallel cyclic dimers, etc. ; see, e.g. PMID 22230563 (stapled peptides), PMID 23064223 (use of click variants for peptide cyclization), PMID 23133740 (optimizing PK properties of cyclic peptides: effects of side chain substitutions), PMID: 22737969 (identification of key backbone motifs for intestinal permeability, PMID 12646037 (cyclization by coupling 2-amino-d,l-dodecanoic acid (Laa) to the N terminus (LaaMII), and by replacing Asn with this lipoamino acid).

In particular embodiments F270 and F274 are substituted and linked, such as wherein the side chains of F270 and F274 replaced by a linker. For example, these residues may be substituted with homocysteines connected through a disulfide bridge to generate a ring and tail cyclic peptide. In addition, the side chains of these residues can be substituted and cross-linked to form a linker, such as —CH2)nONHCOX(CH2)m-, wherein X is C3/4, NH or O, and m and n are integers 1-4, forming a lactam peptide; —CH2OCH2CHCHCH2OCH2-, forming an ether peptide; —(CH2)nCHCH(CH2)m-, forming a stapled peptide. The linkers may incorporate additional atoms, heteroatoms, or other functionalities, and are typically generated from reactive side chain at F270 and F274. The crosslinkable moieties may include additional α-carbon substititions, such as optionally substituted, optionally hetero-lower alkyl, particularly optionally substituted, optionally hetero-methyl, ethyl, propyl and butyl. Suitable modifications include acetylation, acylation, formylation, amidation, phosphorylation (on Ser, Thr and/or Tyr), etc. which can be used to improve peptide bioavailability and/or activity, glycosylation, sulfonation, incorporation of chelators (e.g. DOTA, DPT A), etc. PEGylation can be used to increase peptide solubility, bioavailability, in vivo stability and/or decrease immunogenicity, and includes a variety of different PEGs: HiPEG, branched and forked PEGs, releasable PEGs; heterobifunctional PEG (with endgroup N-Hydroxysuccinimide (NHS) esters, maleimide, vinyl sulfone, pyridyl disulfide, amines, and carboxylic acids), etc.

Suitable terminal modifications include N-terminal acetyl, formyl, myristoyl, palmitoyl, carboxyl and 2-furosyl, and C-terminal hydroxyl, amide, ester and thioester groups, which can make the peptide more closely mimic the charge state in the native protein, and/or make it more stable to degradation from exopeptidases. [038] The peptides may also contain atypical or unnatural amino acids, including D-amino acids, L-homo amino acids, O-β-homo amino acids, N-methylated amino acids, etc.

A wide variety of flanking moieties R1 and/or R2 may be employed, such as affinity tags, transduction domains, homing or targeting moieties, labels, or other functional groups, such as to improve bioavailability and/or activity, and/or provide additional properties.

One useful class of such moieties include transduction domains which facilitate cellular penetrance or uptake, such as protein-derived (e.g. tat, smac, pen, pVEC, bPrPp, PIs1, VP22, M918, pep-3); chimeric (e.g. TP, TP10, MPOA) or synthetic (e.g. MAP, Pep-1, Oligo Arg) cell-penetrating peptides; see, e.g. “Peptides as Drugs: Discovery and Development”, Ed. Bernd Groner, 2009 WILEY-VCH Verlag GmbH & Co, KGaA, Weinheim, esp. Chap 7: “The Internalization Mechanisms and Bioactivity of the Cell-Penetrating Peptides”, Mats Hansen, Elo Eriste, and Ulo Langel, pp. 125-144.

Another class are homing biomolecules, such as RGD-4C, NGR, CREKA, LyP-1, F3, SMS (SMSIARL), IF7, and H2009.1 (Li et al. Bioorg Med Chem. 2011 Sep. 15; 19(18):5480-9), particularly cancer cell homing or targeting biomolecules, wherein suitable examples are known in the art, e.g. Homing peptides as targeted delivery vehicles, Pirjo Laakkonen and Kirsi Vuorinen, Integr. Biol., 2010, 2, 326-337; Mapping of Vascular ZIP Codes by Phage Display, Teesalu T, Sugahara K N, Ruoslahti E., Methods Enzymol. 2012; 503:35-56.

Other useful classes of such moieties include stabilizing agents, such as PEG, oligo-N-methoxyethylglycine (NMEG), albumin, an albumin-binding protein, or an immunoglobulin Fc domain; affinity tags, such as immuno-tags, biotin, lectins, chelators, etc.; labels, such as optical tags (e.g. Au particles, nanodots), chelated lanthanides, fluorescent dyes (e.g. FITC, FAM, rhodamines), FRET acceptor/donors, etc.

The moieties, tags and functional groups may be coupled to the peptide through linkers or spacers known in the art, such as polyglycine, c-aminocaproic, etc.

The compound and/or peptide can also be presented as latent or activatable forms, such as a prodrug, wherein the active peptide is metabolically liberated; for example, release of the linear peptide from cyclic prodrugs prepared with an acyloxyalkoxy promoiety (prodrug 1) or a 3-(2′-hydroxy-4′,6′-dimethylphenyl)-3,3-dimethyl propionic acid promoiety (prodrug 2). of the compound).

According to preferred embodiments of the invention, the molecule for use in the treatment and/or prevention of a bone growth disorder is a Beclin 1 derivative peptide comprising a sequence, unsubstituted, selected from:

SEQ ID NO: 17
VFNATFEIWHD;
SEQ ID NO: 18
CFNATFEIWHD;
SEQ ID NO: 19
VWNATFEIWHD;
SEQ ID NO: 20
VFNATFDIWHD;
SEQ ID NO: 21
VFNATFELWHD;
SEQ ID NO: 22
VFNATFEIFHD;
SEQ ID NO: 23
VFNATFEIWYD;
SEQ ID NO: 24
VFNATFEIWHE;
SEQ ID NO: 25
VWNATFELWHD;
SEQ ID NO: 26
VFNATFEVWHD;
SEQ ID NO: 27
VLNATFEIWHD;
SEQ ID NO: 28
VFNATFEMWHD;
SEQ ID NO: 29
VWNATFHIWHD;
SEQ ID NO: 30
VFNATFEFWHD;
SEQ ID NO: 31
VFNATFEYWHD;
SEQ ID NO: 32
VFNATFERWHD;
SEQ ID NO: 33
FNATFEIWHD;
SEQ ID NO: 34
VFNATFEIWH;
SEQ ID NO: 35
FNATFEIWH;
SEQ ID NO: 36
WNATFHIWH;
SEQ ID NO: 37
VWNATFHIWH;
SEQ ID NO: 38
WNATFHIWHD,

or the D-retro-inverso sequence of said peptides.

According to preferred embodiments of the invention, R1 of said compound comprises a transduction domain, a homing peptide, or a serum stabilizing agent.

According to preferred embodiments of the invention, R1 of said compound is a tat protein transduction domain linked to the peptide through a diglycine linker, particularly a diglycine-T-N linker.

According to preferred embodiments of the invention, R2 of said compound is carboxyl or comprises an affinity tag or detectable label, particularly a fluorescent label.

According to preferred embodiments of the invention F270 and F274 are substituted with cross-linkable moieties and/or linked, and each optionally comprises an additional α-carbon substitution selected from substituted, optionally hetero-lower alkyl, particularly optionally substituted, optionally hetero-methyl, ethyl, propyl and butyl; or F270 and F274 are substituted with homocysteines connected through a disulfide bridge to generate a ring and tail cyclic peptide.

According to preferred embodiments of the invention, the side chains of F270 and F274 are replaced by a linker:

—(CH2)nONHCOX(CH2)m-, wherein X is CH2, NH or O, and m and n are integers 1-4, forming a lactam peptide; —CH2OCH2CHCHCH2OCH2-, forming an ether peptide; or

—(CH2)nCHCH(CH2)m-, forming a stapled peptide.

According to preferred embodiments of the invention, 1 to 6 residues are alanine substituted; or the peptide comprises at least one of substitutions: H275E and S279D; or the peptide comprises one or more D-amino acids, E-β-homo amino acids, O-β-homo amino acids, or N-methylated amino acids; or the peptide comprises the D-retro-inverso sequence.

According to preferred embodiments of the invention, the peptide is acetylated, acylated, formylated, amidated, phosphorylated, sulfated or glycosylated.

According to preferred embodiments of the invention, the compound comprises an N-terminal acetyl, formyl, myristoyl, palmitoyl, carboxyl or 2-furosyl group, and/or a C-terminal hydroxyl, amide, ester or thioester group.

According to preferred embodiments of the invention, the peptide is cyclized.

Preferably, the molecule of the invention for use in the treatment of a bone growth disorder is a peptide of sequence comprising SEQ ID NO: 1 (Tat-Beclin 1), or derivatives thereof, or a polynucleotide encoding for said peptide of sequence comprising SEQ ID NO: 1, or for a derivative thereof.

According to a further preferred embodiment, the molecule of the invention for use in the treatment of a bone growth disorder is a peptide of sequence comprising SEQ ID NO: 2 (retro-inverso Tat-Beclin 1) or derivatives thereof, or a polynucleotide encoding for said peptide of sequence comprising SEQ ID NO: 2 or for a derivative thereof.

According to a preferred embodiment, the molecule of the invention is a vector comprising a polynucleotide encoding for a peptide of sequence SEQ ID NO: 1 or SEQ ID NO:2, or derivatives thereof.

According to a preferred embodiment, the molecule of the invention is a vector comprising an expression cassette, said expression cassette comprising a polynucleotide encoding for any of the Beclin 1 fragment peptides and Beclin 1 derivative peptides disclosed herein; preferably said polynucleotide encodes for a peptide of sequence SEQ ID NO: 1 or SEQ ID NO: 2, or derivatives thereof.

Preferably the polynucleotides encoding for the Beclin 1 fragment peptides and Beclin 1 derivative peptides of the vectors of the present invention are under the control of a regulatory sequence, such as a promoter. Regulatory sequences contemplated for use in said vectors, include but are not limited to, native gene promoters, a cytomegalovirus (CMV) promoter, a liver-specific promoter, and a cartilage-specific promoter. Exemplary liver-specific promoters include human thyroid hormone-globulin (TBG) promoter and alpha-antitrypsin (AAT) promoter. In some embodiments, the promoter is selected from the group consisting of cytomegalovirus (CMV) promoter of sequence SEQ ID No. 39, human thyroid hormone-globulin (TBG) promoter of sequence SEQ ID No. 40, type 2 collagen (Col2A1) promoter of sequence SEQ ID No. 41, and Prrx 1 promoter of sequence SEQ ID No.42.

According to a preferred embodiment of the invention, said vector comprises an expression cassette of sequence SEQ ID NO: 3.

According to a preferred embodiment said vector comprises a polynucleotide of sequence comprising SEQ ID NO:7.

Preferably, the vector of the invention is a viral vector, more preferably a viral vector suitable for gene therapy.

Suitable viruses for expression vectors delivery include retroviruses, lentiviruses, adenoviruses, adeno-associated viruses, herpes viruses, baculoviruses, picornaviruses, and alphaviruses.

According to a preferred embodiment, the molecule of the invention is a viral vector for delivery of an expression vector, said expression vector comprising a polynucleotide coding for an activator of Beclin 1/Vps34 complex; said viral vector is preferably selected from the group of: adenoviral vectors, adeno-associated viral (AAV) vectors, pseudotyped AAV vectors, herpes viral vectors, retroviral vectors, lentiviral vectors, baculoviral vectors. Pseudotyped AAV vectors are those which contain the genome of one AAV serotype in the capsid of a second AAV serotype; for example an AAV2/8 vector contains the AAV8 capsid and the AAV 2 genome. Such vectors are also known as chimeric vectors. The present invention preferably employs adeno-associated viruses (AAV).

Exemplary AAV vectors, for use in embodiments of the present invention, include AAV types 2, 8, 9, 2/1, 2/2, 2/5, 2/7, 2/8, 2/9, rh10, rh39, rh43.

According to a preferred embodiment, a vector according to the invention may be administered to a subject in need thereof at a dose range between 1×10⁹ viral particles (vp)/kg and 1×10¹⁴ vp/kg, a dose range between 1×10¹⁰ vp/kg and 1×10¹³ vp/kg, a dose range between 1×10¹¹ vp/kg and 1×10¹² vp/kg.

Naked plasmid DNA vectors and other vectors known in the art may also be used according to the present invention. Other examples of delivery systems include ex vivo delivery systems, which include but are not limited to DNA transfection methods such as electroporation, DNA biolistics, lipid-mediated transfection, compacted DNA-mediated transfection.

In the present invention polynucleotides or peptides may be isolated. A peptide according to the invention may be a recombinant peptide, obtained by any know methods in the art.

A peptide or a fragment thereof according to the invention may be synthesized via standard methods of synthetic chemistry, i.e. homogeneous chemical syntheses in solution or in solid phase. By way of illustration, those skilled in the art may use the polypeptide solution-synthesis techniques described by Houben Weil (1974, in Methode der Organischen Chemie, E. Wunsh ed., volume 15-1 and 15-11, Thieme, Stuttgart.). A peptide or a fragment thereof according to the invention may also be synthesized chemically in liquid or solid phase by successive coupling of the various amino acid residues (from the N-terminal end to the C-terminal end in the liquid phase, or from the C-terminal end to the N-terminal end in the solid phase). Those skilled in the art may especially use the solid-phase peptide synthesis technique described by Merrifield (Merrifield R. B., (1965a), Nature, vol. 207 (996): 522-523; Merrifield R. B., (1965b), Science, vol. 150 (693): 178-185).

According to another aspect, a peptide, a derivative or a fragment thereof according to the invention may be synthesized by genetic recombination in a host cell and purified, as an example, by the purification techniques described by Molinier-Frenkel (2002, J. Viral. 76, 127-135), by Karayan et al. (1994, Virology 782-795) or by Novelli et al. (1991, Virology 185, 365-376).

During the past decade, gene therapy has been applied to the treatment of disease in hundreds of clinical trials. Various tools have been developed to deliver genes into human cells. In the present invention the delivery vehicles may be administered to a patient. A skilled worker would be able to determine appropriate dosage range. The term “administered” includes delivery by viral or non-viral techniques. Non-viral delivery mechanisms include but are not limited to lipid mediated transfection, liposomes, immunoliposomes, lipofectin, cationic facial amphiphiles (CFAs) and combinations thereof.

The present invention also concerns pharmaceutical compositions comprising the molecule of the invention, optionally in combination with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilizing agent(s), and other carrier agents that may aid or increase the viral entry into the target site (such as for example a lipid delivery system).

Pharmaceutical compositions adapted for topical or parenteral administration, comprising an amount of a compound, constitute a preferred embodiment of the invention. For parenteral administration, the compositions may be best used in the form of a sterile aqueous solution which may contain other substances, for example enough salts or monosaccharides to make the solution isotonic with blood.

The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

In particular, Beclin 1 peptide or a fragment or a derivative thereof may be administered at a dose from 0.001 to 100 mg/kg of body weight, preferably from 0.01 to 50 mg/kg, still preferably from 0.1 to 10 mg/kg, yet preferably from 0.5 to 5 mg/kg, more preferably from 1 to 3 mg/kg.

mTORC inhibitors are administered at a dose from 0.001 to 100 mg/day, preferably from 0.01 to 50 mg/day, still preferably from 0.1 to 10 mg/day, yet preferably from 0.5 a 5 mg/day, more preferably from 1 a 3 mg/day.

The methods of the present invention can be used with humans and other animals. As used herein, the terms “patient” and “subject” are used interchangeably and are intended to include such human and non-human species. Likewise, in vitro methods of the present invention can be earned out on cells of such human and non-human species.

The subject invention also concerns kits comprising the molecule or vector or the host cells of the invention in one or more containers. Kits of the invention can optionally include pharmaceutically acceptable carriers and/or diluents. In one embodiment, a kit of the invention includes one or more other components, adjuncts, or adjuvants as described herein. In one embodiment, a kit of the invention includes instructions or packaging materials that describe how to administer a vector system of the kit. Containers of the kit can be of any suitable material, e.g., glass, plastic, metal, etc., and of any suitable size, shape, or configuration. In one embodiment, the molecule or vector or the host cells of the invention is provided in the kit as a solid. In another embodiment, the molecule or vector or the host cells of the invention is provided in the kit as a liquid or solution. In one embodiment, the kit comprises an ampoule or syringe containing the molecule or vector or the host cells of the invention in liquid or solution form.

The present invention also provides a pharmaceutical composition for treating an individual by gene therapy, wherein the composition comprises a therapeutically effective amount of the molecule of the present invention. Preferably, the gene therapy may be achieved by the administration of a single vector comprising:

i) a polynucleotide coding for any of the molecules of the invention described herein; more preferably a polynucleotide coding for a beclin 1 derivative, more preferably a polynucleotide coding for a Tat-Beclin 1 peptide, or a retro-inverso Tat-Beclin 1 peptide, or derivatives thereof, as herein described; and

ii) a polynucleotide coding for the wild-type form of the protein whose mutated form is responsible for the bone growth disorder.

Alternatively, two vectors may be used, each comprising i) or ii), respectively.

Exemplary protein whose mutated form is responsible for a bone growth disorder include: FGFR3, FGFR1, FGFR2, β-glucocerebrosidase, α-mannosidase, α-fucosidase, α-neuraminidase, Cathepsin-A, UDP-N-acetylglucosamine, N-acetylglucosamine-1-phosphotransferase, Sulfatase modifying factor 1, Cathepsin K, α-L-iduronidase, Iduronate-2-sulfatase, Heparan N-sulfatase, α-N-acetyl glucosaminidase, Acetyl-CoA: α-glucosaminide acetyltransferase, N-acetylglucosamine 6-sulfatase, N-acetylgalactosamine-6-sulfatase, 11-D-galactosidase, N-acetylgalactosamine-4-sulfatase, β-glucuronidase, Hyaluronidase.

The pharmaceutical composition may be for human or animal usage. The vector can be administered in vivo or ex vivo.

Typically, an ordinary skilled clinician will determine the actual dosage which will be most suitable for an individual subject and it will vary with the age, weight and response of the particular individual and administration route. A dose range between 1×10⁹ and 1×10¹⁵ genome copies of each vector/kg, preferentially between 1×10¹⁰ and 1×10¹⁴ genome copies of each vector/kg, more preferentially from 1×10¹¹ and 1×10¹³ are expected to be effective in humans. A preferred dose is 4,5×10¹² genome copies of each vector/kg.

Dosage regimes and effective amounts to be administered can be determined by ordinarily skilled clinicians. Administration may be in the form of a single dose or multiple doses. General methods for performing gene therapy using polynucleotides, expression constructs, and vectors are known in the art (see, for example, Gene Therapy: Principles and Applications, Springer Verlag 1999; and U.S. Pat. Nos. 6,461,606; 6,204,251 and 6,106,826).

The molecules according to the invention can activate the Beclin 1/Vps34 complex either directly, e.g. by interacting with the complex, or indirectly, e.g. by interacting with molecules regulating the complex.

In a further aspect, the invention provides a composition comprising the molecule according to any one of previous claims and pharmaceutically acceptable excipients for use in the treatment of a bone growth disorder.

Preferably the composition further comprises a wild-type form of a protein, whose mutated form is responsible for a lysosomal storage disorder with skeleton involvement; preferably said protein is selected from the group consisting of FGFR3, FGFR1, FGFR2, FGFR4, β-glucocerebrosidase, α-mannosidase, α-fucosidase, α-neuraminidase, Cathepsin-A, UDP-N-acetylglucosamine, N-acetylglucosamine-1-phosphotransferase, Sulfatase modifying factor 1, Cathepsin K, α-L-iduronidase, Iduronate-2-sulfatase, Heparan N-sulfatase, α-N-acetyl glucosaminidase, Acetyl-CoA: α-glucosaminide acetyltransferase, N-acetylglucosamine 6-sulfatase, N-acetylgalactosamine-6-sulfatase, β-D-galactosidase, N-acetylgalactosamine-4-sulfatase, β-glucuronidase, Hyaluronidase. Still preferably the composition further comprises a polynucleotide comprising a nucleotide sequence encoding for a wild-type form of said protein whose mutated form is responsible for said lysosomal storage disorder with skeleton involvement.

In a further aspect the invention provides a method of treatment of bone growth disorder comprising administering to a subject in need thereof a molecule as defined above or a composition as defined above or a vector as defined above.

According to preferred embodiments of the invention the bone growth disorder is selected from the group consisting of: achondroplasia, hypochondroplasia, MPS I, MPS II, MPS IV, MPS VI, MPS VII, MPS IX, Gaucher disease type 3, Gaucher disease type 1, a glycoproteinoses, multiple sulfatase deficiency, a pycnodysostosis and a spondyloepiphyseal dysplasia; more preferably, the bone growth disorder is selected from the group consisting of achondroplasia, MPS VI, MPS VII.

Sequences
(Tat-Beclin 1)
SEQ ID NO: 1
YGRKKRRQRRRGGTNVFNATFEIWHDGEFGT
(retro-inverso Tat-Beclin 1)
SEQ ID NO: 2
RRRQRRKKRGYGGTGFEGDHWIEFTANFVNT
(AAV-Beclin 1)
SEQ ID NO: 3
ctgcgcgctcgctcgctcactgaggccgcccgggcaaagcccgggcgtcgggcgacctttggtcgc
ccggcctcagtgagcgagcgagcgcgcagagagggagtggccaactccatcactaggggttccttg
tagttaatgattaacccgccatgctacttatctacgtagccatgctctaggaagatcggaattcgc
ccttaagctagctagttattaatagtaatcaattacggggtcattagttcatagcccatatatgga
gttccgcgttacataacttacggtaaatggcccgcctggctgaccgcccaacgacccccgcccatt
gacgtcaataatgacgtatgttcccatagtaacgccaatagggactttccattgacgtcaatgggt
ggagtatttacggtaaactgcccacttggcagtacatcaagtgtatcatatgccaagtacgccccc
tattgacgtcaatgacggtaaatggcccgcctggcattatgcccagtacatgaccttatgggactt
tcctacttggcagtacatctacgtattagtcatcgctattaccatggtgatgcggttttggcagta
catcaatgggcgtggatagcggtttgactcacggggatttccaagtctccaccccattgacgtcaa
tgggagtttgttttggcaccaaaatcaacgggactttccaaaatgtcgtaacaactccgccccatt
gacgcaaatgggcggtaggcgtgtacggtgggaggtctatataagcagagctggtttagtgaaccg
tcagatcctgcagaagttggtcgtgaggcactgggcaggtaagtatcaaggttacaagacaggttt
aaggagaccaatagaaactgggcttgtcgagacagagaagactcttgcgtttctgataggcaccta
ttggtcttactgacatccactttgcctttctctccacaggtgtccaggcggccgccatggtcagct
actgggacaccggggtcctgctgtgcgcgctgctcagctgtctgcttctcacaggatctagttcag
gttacggccggaagaagcggcggcagcggcggcggggcggcaccaacgtgttcaacgccaccttcc
acatctggcacagcggccagttcggcaccggatccgactacaaagaccatgacggtgattataaag
atcatgacatcgactacaaggatgacgatgacaagtgaaagcttaaaaaaatcaacctctggatta
caaaatttgtgaaagattgactggtattcttaactatgttgctccttttacgctatgtggatacgc
tgctttaatgcctttgtatcatgctattgcttcccgtatggctttcattttctcctccttgtataa
atcctggttgctgtctctttatgaggagttgtggcccgttgtcaggcaacgtggcgtggtgtgcac
tgtgtttgctgacgcaacccccactggttggggcattgccaccacctgtcagctcctttccgggac
tttcgctttccccctccctattgccacggcggaactcatcgccgcctgccttgcccgctgctggac
aggggctcggctgttgggcactgacaattccgtggtgttgtcggggaaatcatcgtcctttccttg
gctgctcgcctgtgttgccacctggattctgcgcgggacgtccttctgctacgtcccttcggccct
caatccagcggaccttccttcccgcggcctgctgccggctctgcggcctcttccgcgtcttcgaga
tctgcctcgactgtgccttctagttgccagccatctgttgtttgcccctcccccgtgccttccttg
accctggaaggtgccactcccactgtcctttcctaataaaatgaggaaattgcatcgcattgtctg
agtaggtgtcattctattctggggggtggggtggggcaggacagcaagggggaggattgggaagac
aatagcaggcatgctggggactcgagttaagggcgaattcccgataaggatcttcctagagcatgg
ctacgtagataagtagcatggcgggttaatcattaactacaaggaacccctagtgatggagttggc
cactccctctctgcgcgctcgctcgctcactgaggccgggcgaccaaaggtcgcccgacgcccggg
ctttgcccgggcggcctcagtgagcgagcgagcgcgcag
(5′-ITR)
SEQ ID NO: 4
ctgcgcgctcgctcgctcactgaggccgcccgggcaaagcccgggcgtcgggcgacctttggtcgc
ccggcctcagtgagcgagcgagcgcgcagagagggagtggccaactccatcactaggggttcct
(CMV promoter + SV40 intron)
SEQ ID NO: 5
Tagttattaatagtaatcaattacggggtcattagttcatagcccatatatggagttccgcgttac
ataacttacggtaaatggcccgcctggctgaccgcccaacgacccccgcccattgacgtcaataat
gacgtatgttcccatagtaacgccaatagggactttccattgacgtcaatgggtggagtatttacg
gtaaactgcccacttggcagtacatcaagtgtatcatatgccaagtacgccccctattgacgtcaa
tgacggtaaatggcccgcctggcattatgcccagtacatgaccttatgggactttcctacttggca
gtacatctacgtattagtcatcgctattaccatggtgatgcggttttggcagtacatcaatgggcg
tggatagcggtttgactcacggggatttccaagtctccaccccattgacgtcaatgggagtttgtt
ttggcaccaaaatcaacgggactttccaaaatgtcgtaacaactccgccccattgacgcaaatggg
cggtaggcgtgtacggtgggaggtctatataagcagagctggtttagtgaaccgtcagatcctgca
gaagttggtcgtgaggcactgggcaggtaagtatcaaggttacaagacaggtttaaggagaccaat
agaaactgggcttgtcgagacagagaagactcttgcgtttctgataggcacctattggtcttactg
acatccactttgcctttctctccacag
(sFLT1)
SEQ ID NO: 6
atggtcagctactgggacaccggggtcctgctgtgcgcgctgctcagctgtctgcttctcacagga
tctagttcaggt
(TAT-Beclin 1 polynucleotide)
SEQ ID NO: 7
Tacggccggaagaagcggcggcagcggcggcggggcggcaccaacgtgttcaacgccaccttccac
atctggcacagcggccagttcggcacc
(3xflag)
SEQ ID NO: 8
gactacaaagaccatgacggtgattataaagatcatgacatcgactacaaggatgacgatgacaag
(WPRE)
SEQ ID NO: 9
Aatcaacctctggattacaaaatttgtgaaagattgactggtattcttaactatgttgctcctttt
acgctatgtggatacgctgctttaatgcctttgtatcatgctattgcttcccgtatggctttcatt
ttctcctccttgtataaatcctggttgctgtctctttatgaggagttgtggcccgttgtcaggcaa
cgtggcgtggtgtgcactgtgtttgctgacgcaacccccactggttggggcattgccaccacctgt
cagctcctttccgggactttcgctttccccctccctattgccacggcggaactcatcgccgcctgc
cttgcccgctgctggacaggggctcggctgttgggcactgacaattccgtggtgttgtcggggaaa
tcatcgtcctttccttggctgctcgcctgtgttgccacctggattctgcgcgggacgtccttctgc
tacgtcccttcggccctcaatccagcggaccttccttcccgcggcctgctgccggctctgcggcct
cttccgcgtcttcg
(BGH polyA)
SEQ ID NO: 10
Gcctcgactgtgccttctagttgccagccatctgttgtttgcccctcccccgtgccttccttgacc
ctggaaggtgccactcccactgtcctttcctaataaaatgaggaaattgcatcgcattgtctgagt
aggtgtcattctattctggggggtggggtggggcaggacagcaagggggaggattgggaagacaat
agcaggcatgctgggga
(3′-ITR)
SEQ ID NO: 11
aggaacccctagtgatggagttggccactccctctctgcgcgctcgctcgctcactgaggccgggc
gaccaaaggtcgcccgacgcccgggctttgcccgggcggcctcagtgagcgagcgagcgcgcag
(Retro-inverso short Tat-Beclin 1)
SEQ ID NO: 12
RRQRRKKKRGYGGDHWIEFTANFV
(Beclin 1 residues 269-283)
SEQ ID NO: 13
VFNATFHIWHSGQFG
(Beclin 1 residues 269-283 N-terminally flanked with TN and C-
terminally flanked by T)
SEQ ID NO: 14
TNVFNATFHIWHSGQFGT
(Beclin 1 residues 269-283 comprising substitutions: H275E,
S279D and Q281E)
SEQ ID NO: 15
VFNATFEIWHDGEFG
(Beclin 1 residues 270-278)
SEQ ID NO: 16
FNATFHIWH
SEQ ID NO: 17
VFNATFEIWHD
SEQ ID NO: 18
CFNATFEIWHD
SEQ ID NO: 19
VWNATFEIWHD
SEQ ID NO: 20
VFNATFDIWHD
SEQ ID NO: 21
VFNATFELWHD
SEQ ID NO: 22
VFNATFEIFHD
SEQ ID NO: 23
VFNATFEIWYD
SEQ ID NO: 24
VFNATFEIWHE
SEQ ID NO: 25
VWNATFELWHD
SEQ ID NO: 26
VFNATFEVWHD
SEQ ID NO: 27
VLNATFEIWHD
SEQ ID NO: 28
VFNATFEMWHD
SEQ ID NO: 29
VWNATFHIWHD
SEQ ID NO: 30
VFNATFEFWHD
SEQ ID NO: 31
VFNATFEYWHD
SEQ ID NO: 32
VFNATFERWHD
SEQ ID NO: 33
FNATFEIWHD
SEQ ID NO: 34
VFNATFEIWH
SEQ ID NO: 35
FNATFEIWH
SEQ ID NO: 36
WNATFHIWH
SEQ ID NO: 37
VWNATFHIWH
SEQ ID NO: 38
WNATFHIWHD
(CMV promoter)
SEQ ID No. 39
TAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAAT
GACGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTCCGCCCCCTATTGACGTCAA
TGACGGTAAATGGCCCGCCTGGCATTATGCCCAGTACATGACCTTACGGGACTTTCCTACTTGGCA
GTACATCTACGTATTAGTCATCGCTATTACCATGGTGATGCGGTTTTGGCAGTACACCAATGGGCG
TGGATAGCGGTTTGACTCACGGGGATTTCCAAGTCTCCACCCCATTGACGTCAATGGGAGTTTGTT
TTGGCACCAAAATCAACGGGACTTTCCAAAATGTCGTAATAACCCCGCCCCGTTGACGCAAATGGG
CGGTAGGCGTGTACGGTGGGAGGTCTATATAAGCAGAGCTCGTTTAGTGAACCGT
(TBG promoter)
SEQ ID No. 40
GCTAGCAGGTTAATTTTTAAAAAGCAGTCAAAAGTCCAAGTGGCCCTTGGCAGCATTTACTCTCTC
TGTTTGCTCTGGTTAATAATCTCAGGAGCACAAACATTCCAGATCCAGGTTAATTTTTAAAAAGCA
GTCAAAAGTCCAAGTGGCCCTTGGCAGCATTTACTCTCTCTGTTTGCTCTGGTTAATAATCTCAGG
AGCACAAACATTCCAGATCCGGCGCGCCAGGGCTGGAAGCTACCTTTGACATCATTTCCTCTGCGA
ATGCATGTATAATTTCTACAGAACCTATTAGAAAGGATCACCCAGCCTCTGCTTTTGTACAACTTT
CCCTTAAAAAACTGCCAATTCCACTGCTGTTTGGCCCAATAGTGAGAACTTTTTCCTGCTGCCTCT
TGGTGCTTTTGCCTATGGCCCCTATTCTGCCTGCTGAAGACACTCTTGCCAGCATGGACTTAAACC
CCTCCAGCTCTGACAATCCTCTTTCTCTTTTGTTTTACATGAAGGGTCTGGCAGCCAAAGCAATCA
CTCAAAGTTCAAACCTTATCATTTTTTGCTTTGTTCCTCTTGGCCTTGGTTTTGTACATCAGCTTT
GAAAATACCATCCCAGGGTTAATGCTGGGGTTAATTTATAACTAAGAGTGCTCTAGTTTTGCAATA
CAGGACATGCTATAAAAATGGAAAGATGTTGCTTTCTGAGAGACTGCAG
(Col2A1 promoter)
SEQ ID No. 41
CACCTTCACACAGGTCTCCTTCTGTGCAGTAACACACCAGCTCTTTTCCTGGCTGTCGGCTCAGGC
CAACTTCGGCCTGTGCTCCAGAGGAAGCCTTCAACGCAGAGCTGGATGGGGGAGGGGTGGAGGGCA
GTCGCTGTGAACGTCCAGGTGGGAGTCTGGGGACCAGGTACTGCAGGGAAGGGCTAAAAGATAGGT
CGGGGTAACCCTTCAGATCTGGCTCAGCTAGCCTGTCTCCAAGATTTAGGACTCTGAATCTCTGTG
GGCTCCTCCCTGTCCCCACTCCCAAACGCCTGACGCGGTGCCCCCTCGCCCTCCGCTGCTCCTTTC
TACCGCTTTCCCTCCTCCCTCCCATGTCTTTTCCGTCCTTGGTCTAGGGCTCTCGGCCTGCGCCTC
TGCAAACACCCCCTCCCCTCCAACTCCGGCAGAACTCCGAGGGGAGGGGCCGGAGGCCACCCTTCC
CGCCTGTGGTCAGAGGGGGGCAGCGCCGCAGCCCCGGGTTTGGGGGGCAGGGGCCATCTCTGCGCC
CCGCCCGATCAGGCCACTCGGCGCACTAGGGGTGGAGGGCGGGAAGCGTGACTCCCAGAGAGGGGG
GTCCGGCTTGGGCAGGTGCGGGCACTGGCAGGGCCCAGGCGGGCTCCGGGGGCGGGCGGTTCAGGT
TACAGCCCAGCGGGGGGCAGGGGGCGGCCCGCGGTTTGGGCGAGTTCGCCAGCCTCGAAAGGGGCC
GGGCGCATATAACGGGCGCCGCGGCGGGGAGAAGACGCAGAGCGCTGCTGGGCTGCCGGGTCTCCC
GCTTCCCCCTCCTGCTCCAAGGGCCTCCTGCATGAGGGCGCGGTAGAG
(Prrx 1 promoter)
SEQ ID No. 42
GCTTCTTGATCCAACTGAGAAGGAAAAAGGAGCCCAGCAAGAAGAGGGGGAGAGAGAGAAGGGGAA
AGGGGGGAACCCACCAGCACCCTCCGTCGGACTCTTGAAGCCTTTTTTTTTTAATTCTTAATTTTT
TTTTTTACTCTTTACAAAAAGTAAAGTGAGAATCCTGCTCTCTAATACATCTGCAAGACATCACCC
TCTCCTCCTGAAACTTTAGTCACTCCTGAGAATCCACAGGAGTGCAGAGAGGGGGGAACACGTTTT
CTTGAAGATGTTTTAAAGCTGGAACAAGCCTTCTTCTGTTGGTGCTTGAACTCTTGCCTGGGAATA
ACTTTTTTAACCTTTAAAAAAACCATTCACTTTGATTCTTCTCTCCCACCCCTTCTTCTCTCTTCT
TCTGTTTGCCTAACTCCCCCGCCCTGCTGGCCTCCGCTTTCCTCTCTCCCCCTTGTTATTATTTTT
AGTCTGTGCGTGTGGACACTTTTGGAGAGTTGGAAGGGATTTTTTTCTCCTGACTTGAACATAGGG
TGACTTTTTAATATTGTATTTTACTGTGGATTATCTCTTTGGACCGCGCCGGACTTGGCCTCAGGA
AATCAACCAATGCTGCGGAAGGCGGCTGGTGCACAACGCTCTGCTCTACAGAAGGGGGTCCCCCAC
CCTCTTTTCCAATTTTTTTTTTTTGGCCTTCCTCTCCTTCCCTCCCTCTTCCTCCCTCTCTCTCTC
TCTCTCTCCACTACCCCCCTCTTTCTTCCCCACTCGGCTCCTCTCCCCCCTCGCGCCCACAGCGTT
TGGTGTTGATTCGAGCGGGAAGAGGGGGGTGGGTGGGATCGGTGGGGGAGACCATGACCTCCAGCT
ACGGGCACGTTCTGGAGCGGCAACCGGCGCTGGGCGGCCGCTTGGACAGCCCGGGCAACCTCGACA
CCCTGCAGGCGAAAAAGAACTTCTCCGT
SEQ ID No. 43
MEGSKTSNNSTMQVSFVCQRCSQPLKLDTSFKILDRVTIQELTAPLLTTAQAKPGETQEEETNSGE
EPFIETPRQDGVSRRFIPPARMMSTESANSFTLIGEASDGGTMENLSRRLKVTGDLFDIMSGQTDV
DHPLCEECTDTLLDQLDTQLNVTENECQNYKRCLEILEQMNEDDSEQLQMELKELALEEERLIQEL
EDVEKNRKIVAENLEKVQAEAERLDQEEAQYQREYSEFKRQQLELDDELKSVENQMRYAQTQLDKL
KKTNVFNATFHIWHSGQFGTINNFRLGRLPSVPVEWNEINAAWGQTVLLLHALANKMGLKFQRYRL
VPYGNHSYLESLTDKSKELPLYCSGGLRFFWDNKFDHAMVAFLDCVQQFKEEVEKGETRFCLPYRM
DVEKGKIEDTGGSGGSYSIKTQFNSEEQWTKALKFMLTNLKWGLAWVSSQFYNK
(TAT moiety)
SEQ ID No. 44
YGRKKRRQRRR
(retro-inverso TAT moiety)
SEQ ID No. 45
RRRQRRKKRGY

### EXAMPLES

### Example 1—Modulation of Autophagy Prevents the Skeletal Defects Associated with LSDs

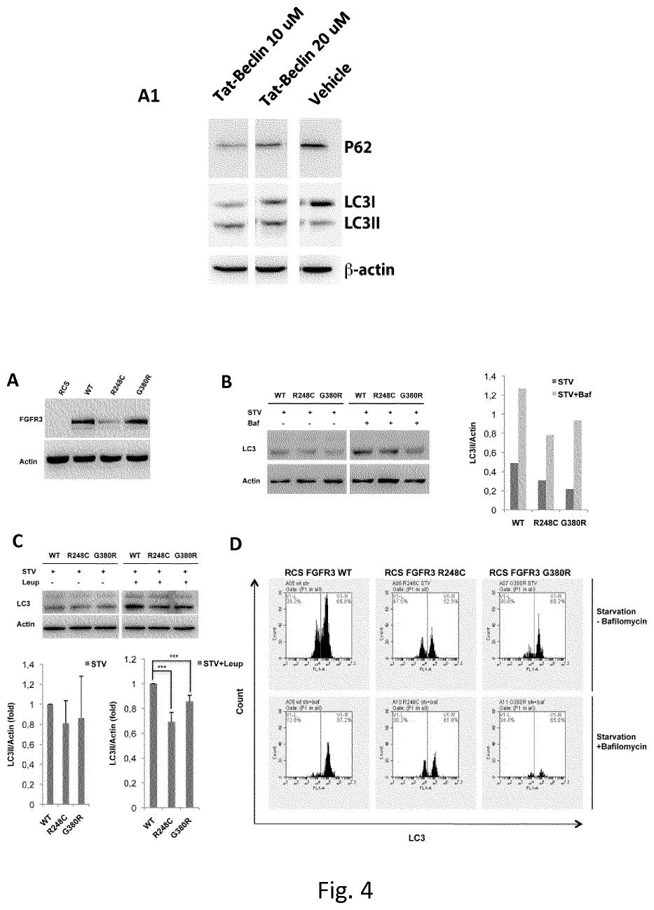

Tat-Beclin 1 peptide is capable of inducing autophagy in a cell by activating Beclin 1-Vps34 complex (see FIG. 4A1).

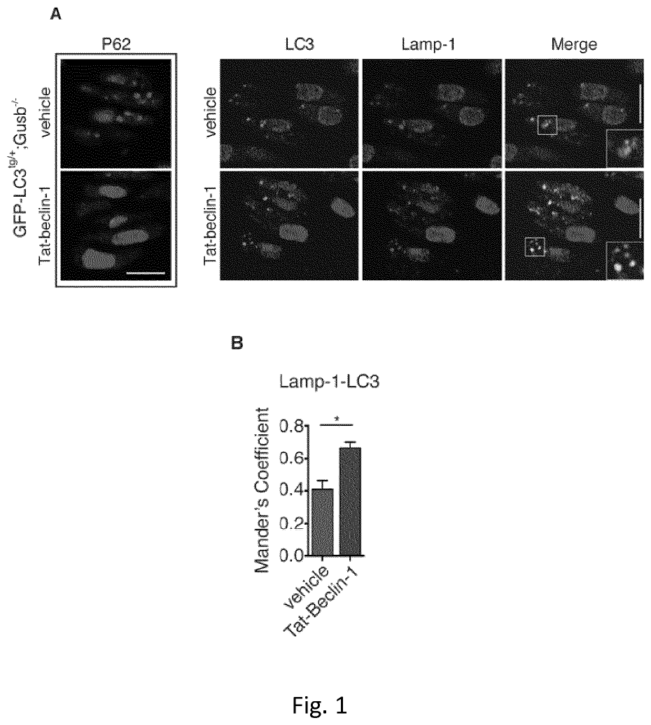

Daily injection of Tat-Beclin1 peptide promoted Av-Lys fusion and p62/SQSTM1 degradation in the growth plate of MPS VII (Gusb−/−) mice expressing the fluorescent autophagy reporter GFP-LC3⁶⁴ (Gush−/−; GFP-LC3^{tg/+} mice) (FIG. 1 a,b).

Newborn MPS VII and MPS VI mice were intraperitoneally injected daily with retro-inverso Tat-Beclin 1 peptide (Beclin 1Activator II, retro-inverso Tat-Beclin 1, Millipore) at 2 mg/kg resuspended in PBS, according to a preferred embodiment of the invention. Control mice were injected with vehicle only. Mice were sacrificed after 15 (P15) and 30 (P30) days.

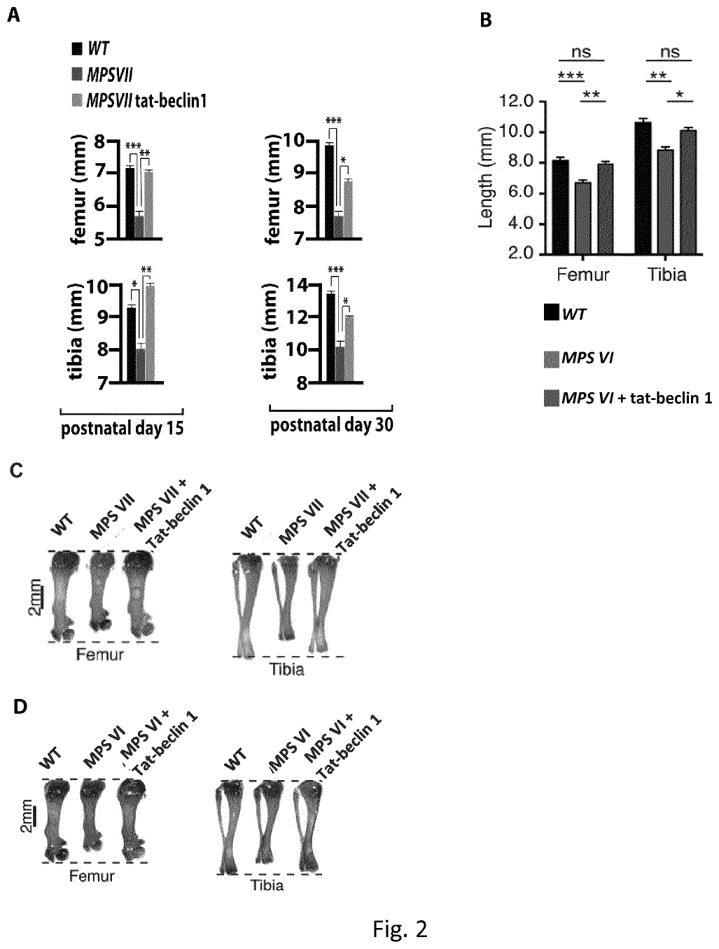

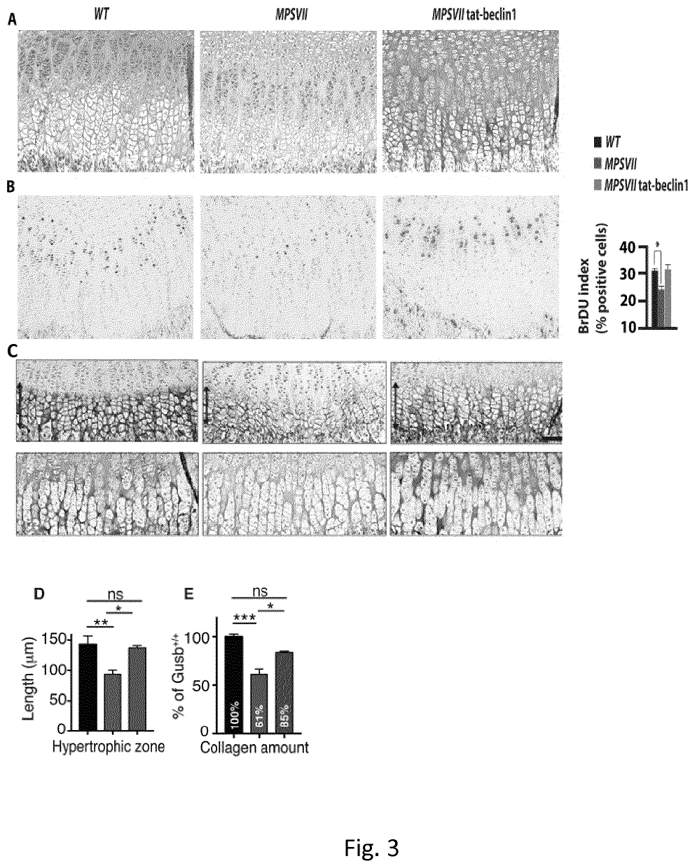

Starting at postnatal day 15 (P15) MPSVII mice show significant reduced femur and tibia lengths compared to wild type mice (FIG. 2a,c). A similar phenotype was also observed in P15 MPSVI mice (FIG. 2b, d), indicating that inventors findings can be also extended to other MPSs. Histological analysis of femoral and tibial growth plates from P15 MPSVII mice showed altered architecture and shorter length of pre-hypertrophic and hypertrophic zones compared to wild type mice (FIG. 3a). Rate of chondrocytes proliferation was decreased and type X collagen (Col10) narrowed in MPSVII mice growth plates compared to wild type mice (FIG. 3b,c), suggesting defective chondrocyte proliferation and differentiation in MPSVII mice. In vivo, intraperitoneal (i.p.) injection of the retro-inverso Tat-Beclin 1 peptide, according to a preferred embodiment of the invention, rescues femoral and tibial growth retardation in MPSVII and MPSVI mice (FIG. 2a,b,c,d) and normalizes growth plate differentiation and proliferation defects and collagen levels in femoral and tibia cartilages in MPSVII mice (FIG. 3a-e).

### Example 2—FGR3^{ach} and FGFR^{TD} Chondrocytes Show Inhibited Autophagy Flux

RCS cells stably expressing FGFR3 wild-type (wt), R248C (FGFR3^{TD}) and G380R (FGFR3^{ach}) mutations, associated to achondroplasia in humans, were prepared by retroviral transduction. FGR3^{ach} and FGFR^{TD} chondrocytes were treated with lysosomal inhibitors leupeptin and bafilomycin to clamp autophagosomes (AVs) degradation. Leupeptin and bafilomycin treatments did not increase the level of LC3II protein in FGR3^{ach} and FGFR^{TD} chondrocytes, compared to FGFR3 wild type stable cells (FIG. 4a-c). Fluorescent Activated Cell Sorting (FACS) analysis also show a decreased level of endogenous LC3 fluorescence in FGR3^{ach} and FGFR^{TD} stable chondrocytes cell line compared to wild type FGFR3 chondrocytes (FIG. 4d).

### Materials and Methods of Examples 1-2

Animals: MPSVI (Arsb^{-/-}) mice^{59,50} obtained from A. Auricchio (Telethon Institute of Genetics and Medicine, TIGEM, Naples). MPSVII mice (Gusb^{-/-})⁵⁸ were obtained from Jackson Laboratories. All mice used were maintained in a C57BL/6 strain background. Experiments were conducted in accordance with the guidelines of the Animal Care and Use Committee of Cardarelli Hospital in Naples and authorized by the Italian Ministry of Health. Tissues and histology: Histology was performed according to standardized procedures (http://empress.har.mrc.ac.uk/browser/). Briefly, femurs were fixed ON in 4% (wt/vol) paraformaldehyde (PFA) and then demineralized in 10% EDTA (pH 7.4) for 48h. Specimens were then dehydrated, embedded in paraffin and sectioned at 7 μm, and stained with hematoxylin and eosin. For BrDU staining mice were injected with 200 μl of 10 mM BrDU (Sigma) 4h before sacrifice. BrDU incorporation was detected using a Zymed BrDU staining kit (Invitrogen). Counterstaining was performed using hematoxylin Immunohistochemistry were performed according to standardized protocols. Briefly, type X collagen (Hybridoma bank) staining were performed pretreating paraffin-embedded sections with 1 mg/ml pepsin in 0.1 M Acetic Acid, 0.5 M NaCl for 2 h at 37° C., and then treated with 2 mg/ml hyaluronidase in 0.1 M TBS for 1 h at 37° C., prior to the blocking step. Endogenous peroxidases were quenched with 3% hydrogen peroxide, sections were then incubated with blocking serum and primary antibody over night at 4° C. Signals were developed using Vectastain Elite ABC kit (Vector Laboratories) and NovaRED Peroxidase Substrate kit (Vector Laboratories).

Western blotting: Cells were washed twice with PBS and then scraped in lysis buffer (RIPA lysis buffer in the presence of PhosSTOP and EDTA-free protease inhibitor tablets—Roche, Indianapolis, Ind., USA). Cell lysates were incubated on ice for 20′, then the soluble fraction was isolated by centrifugation at 14,000 rpm for 10 mM at 4° C. Total protein concentration in cellular extracts was measured using the colorimetric BCA protein assay kit (Pierce Chemical Co, Boston, Mass., USA). Protein extracts, separated by SDS-PAGE and transferred onto PVDF membrane, were probed with antibodies against LC3, β-actin (Novus Biologicals), P62 (Abnova) and FGFR3 (Cell Signaling). Proteins of interest were detected with HRP-conjugated goat anti-mouse or anti-rabbit IgG antibody (1:2000, Vector Laboratories) and visualized with the Super Signal West Dura substrate (Thermo Scientific, Rockford, Ill.), according to the manufacturer's protocol. The Western blotting images were acquired using the Chemidoc-lt imaging system (UVP) and band intensity was calculated using imageJ software using “Gels and Plot lanes” plug-in.

Retrovirus preparation: Retroviral particles were produced using packaging plasmids (VSV-G and gag/pol) (Addgene) in 293T cells (ATCC, Manassas, Va.). 293T were cultured in DMEM containing 10% FBS and were transfected using Lipofectamine LTX and Plus reagent (Invitrogen). The supernatant containing retroviral particles was collected after 48-72 hours for RCS transduction and filtered through 0.45 mm filter (Corning). Infected RCS cells were selected with puromycin (2.5 μg/mL). Plasmids: pBp-FGFR3c-wt and pBp-FGFR3c-R248C were purchased from Addgene; pBp-FGFR3c-G380R was generated using QuickChange Site-Directed Mutagenesis Kit (Agilent Technologies).

Leupeptin and bafilomycin treatments: Leupeptin (Sigma) was resuspended in water at 10 mM. FGFR3 wild type, FGFR3ach and FGFR3TD stable chondrocytes cell line were treated with 50 μM leupeptin for 2 h at 37° C. Bafilomycin (Millipore) was resuspended in DMSO at 200 μM. FGFR3 wild type, FGFR3 ach and FGFR3TD stable chondrocytes cell line were treated with 200 nM bafilomycin for 4 h at 37° C.

FACS: RCS cells stably expressing FGFR3 WT, R248C and G380R were harvested in trypsin, washed with PBS, fixed for 10 mM in ice-cold methanol and permeabilized for 15 min with 100 μg/mL digitonin in PBS. Cells were then incubated with mouse anti-LC3 primary antibody (Nanotools) for 30 min, washed three times in PBS, and incubated for 30 mM with goat anti-mouse secondary antibody (Alexa labelled). FACS data were collected using BD Accuri C6 Cytometer (BD Biosciences) and data analysis was carried out with BD Accuri C6 Software.

### Example 3—Autophagy Flux Increases During Early Post-Natal Bone Development

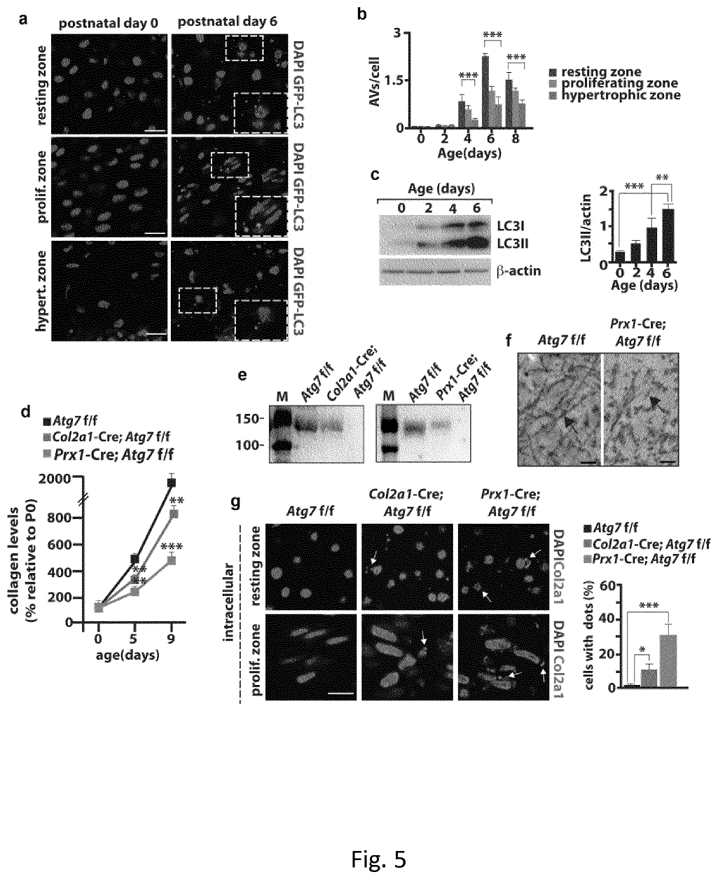

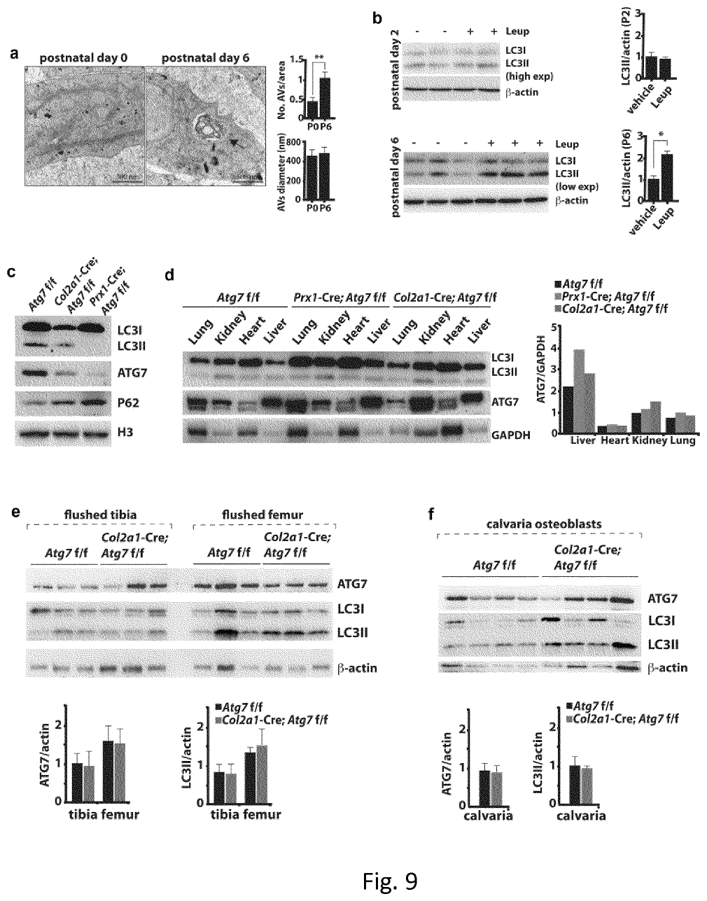

The femoral growth plates of mice that ubiquitously express the autophagosome marker MAP1LC3 tagged with green fluorescent protein (GFP) (GFP-LC3tg/+) (Mizushima N et al, Mol Biol Cell 2004) were analyzed. Very few autophagic vesicles (AVs) were detected in the growth plates of newborn mice (P0) (FIG. 5a, quantification in 5b). Sections obtained from older mice (P2 to P8) showed a progressive age-dependent increase in the number of AVs (FIG. 5a, quantification in 5b). This observation was confirmed by TEM analysis (FIG. 9a) and biochemically by quantifying the conversion of the non-lipidated form of LC3 (LC3I) to the autophagosome-associated lipidated form (LC3II) (Kabeya Y et al., 2005) in femoral growth plates of wild type mice at different time points (FIG. 5c). In vivo inhibition of lysosomal function by Leupeptin administration further increased the levels of LC3II in the growth plate of P6 but not P2 mice, indicating that the autophagic flux is enhanced in P6 growth plate chondrocytes (FIG. 9b).

### Example 4—Autophagy Regulates Skeletogenesis and the Composition of Growth Plate ECM

The essential autophagy gene 7 (Atg7) was deleted in chondrocytes by crossing a mouse line carrying the Atg7 floxed allele (Atg7f/f) (Komatsu, M. et al., J. Cell Biol. 2005) with two different Cre mouse lines: 1) the Prx1-Cre line, in which the Cre protein is expressed in the mesenchymal cells of the limbs during embryogenesis (Logan M et al., Genes 2002) and 2) the Col2a1-Cre line, in which the expression of the Cre protein is mainly restricted to mature chondrocytes before and after birth (Ovchinnikov DA, Genes 2002).

The selective lack of Atg7 protein and the inhibition of functional autophagy in the femoral growth plates of Atg7f/f; Prx1-Cre and Atg7f/f; Col2a1-Cre mice was verified (FIG. 9c-f).

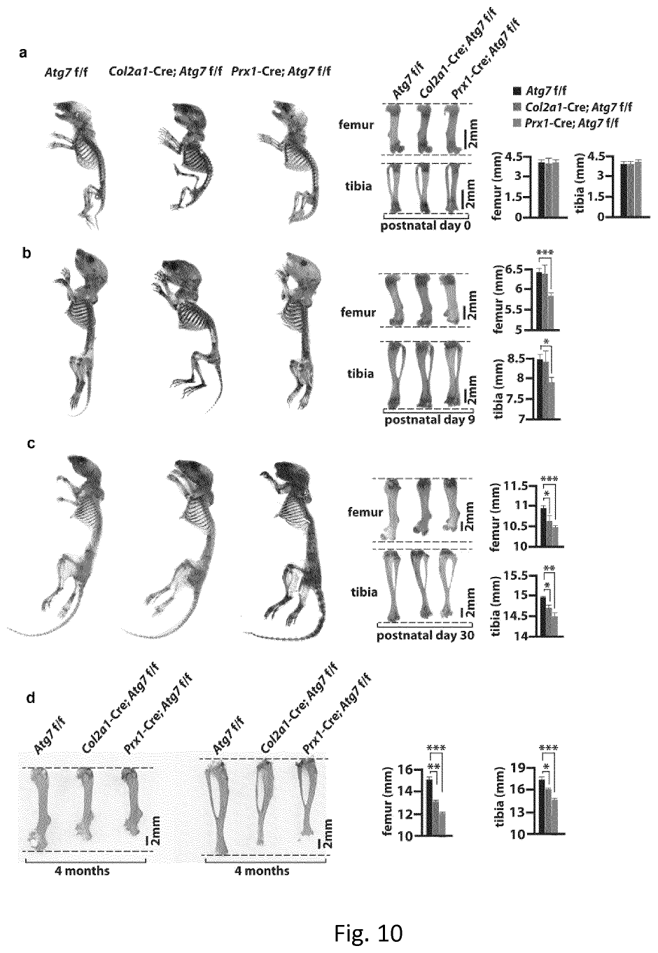

Atg7f/f; Prx1-Cre and Atg7f/f; Col2a1-Cre mice were born at the expected Mendelian ratio, with bones of normal shapes and sizes, suggesting that chondrocyte autophagy is dispensable during embryonic skeletal development (FIG. 10a). However, starting at P9 the Atg7f/f; Prx1-Cre mice showed reduced femoral and tibia lengths compared to control mice (FIG. 10b). A similar, albeit milder, phenotype was also observed in Atg7f/f; Col2a1-Cre mice (FIG. 10c,d).

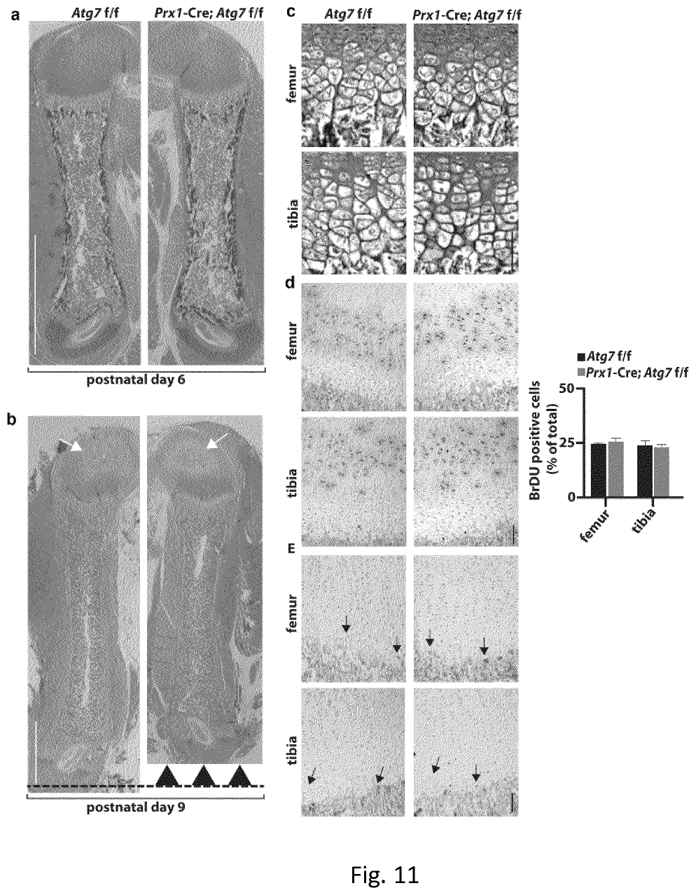

Histological analyses of femoral and tibia growth plates from P6 and P9 Atg7f/f; Prx1-Cre and Atg7f/f; Col2a1-Cre mice showed preserved architecture and normal rates of chondrocyte differentiation, proliferation and terminal apoptosis, suggesting that these processes occur independently of autophagy in chondrocytes (FIG. 11a-e).

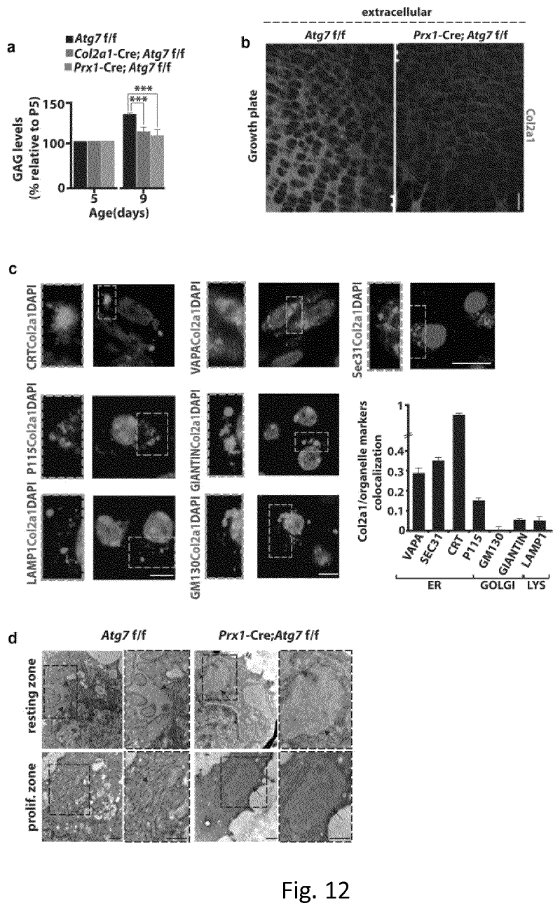

The levels of glycosaminoglycans, were only slightly reduced in the growth plate of Atg7f/f; Prx1-Cre and Atg7f/f; Col2a1-Cre mice compared to controls (FIG. 12a). Type II procollagen (PC2) is the main protein synthesized in chondrocytes, and type II collagen (Col2) constitutes the majority of cartilaginous ECM (Olsen, B. R. et al., Annu. Rev. Cell Dev. Biol. 2000). Col2 levels were normal in the growth plates of Atg7f/f; Prx1-Cre and Atg7f/f; Col2a1-Cre mice at birth, but did not increase during post-natal growth contrary to that observed in control mice (FIG. 5d,e and FIG. 12b). Consistently, transmission electron microscopy (TEM) of femoral growth plate sections isolated from Atg7f/f; Prx1-Cre mice at P6 showed a sparse and disorganized interterritorial Colt fibril network (FIG. 50. These data suggest that autophagy regulates post-natal bone growth in part by controlling the levels of Col2 deposited by chondrocytes in the growth plate ECM.

By using an antibody that recognizes the pro-alpha1(II) chain of Col2/PC2 proteins (Col2a1), accumulation of Col2a1 molecules in the ER of chondrocytes lacking autophagy (FIG. 5g,h) was observed. No colocalization of Col2a1 was observed with other organelle markers (FIG. 12c,d). Consistently, TEM analysis showed that the ER cisternae of Atg7f/f; Prx1-Cre chondrocytes were enlarged and filled with electron dense material (FIG. 5i).

### Example 5—Autophagy Regulates PC2 Secretion

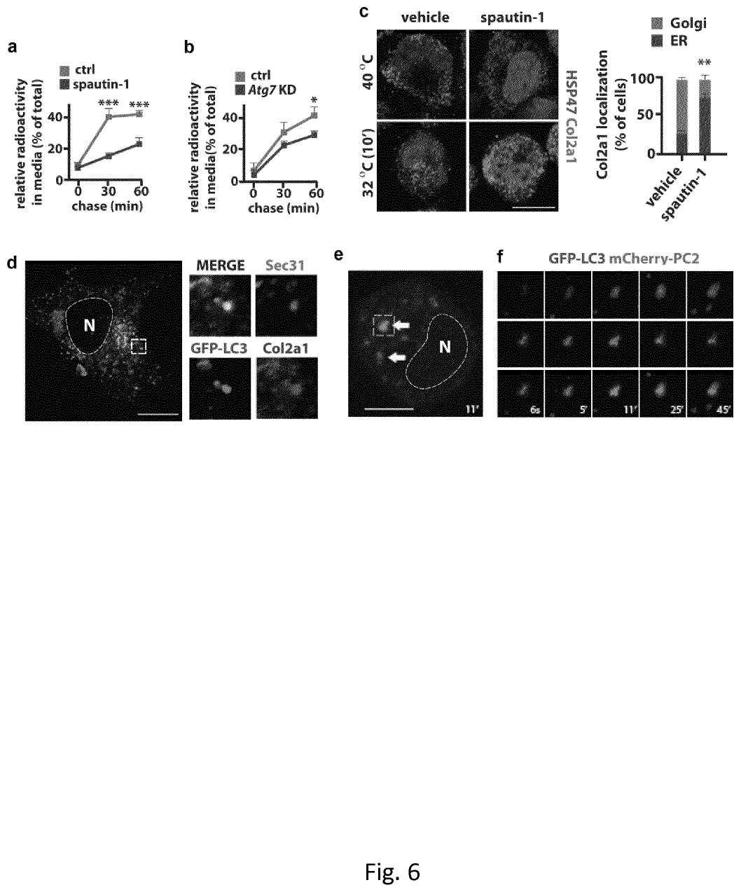

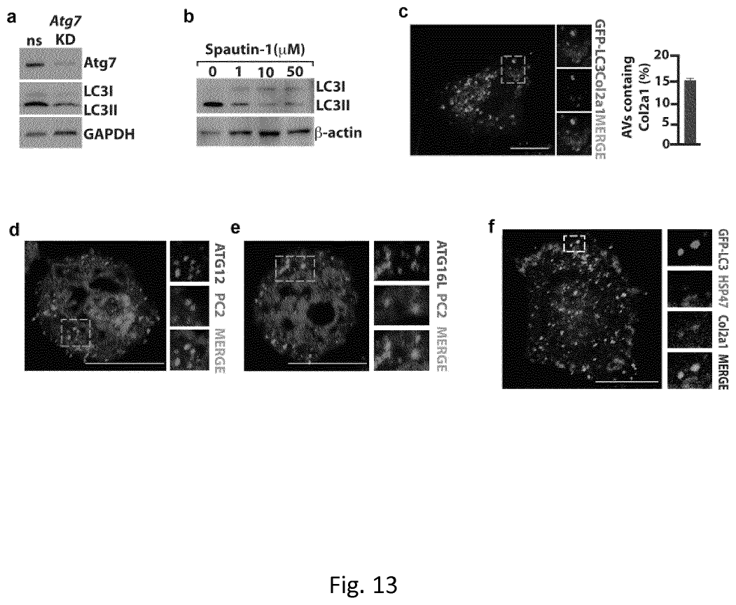

The inhibition of autophagy with Spautin-1 (Liu, J. et al., Cell 2011) or with RNA interference targeting Atg7(Atg7Kd) in cultured Rx chondrocytes in which PC2 secretion was synchronized (Venditti R et al., Science 2012) led to defective PC2 secretion and to the retention of PC2 in the ER (FIG. 6a-c and FIG. 13a,b). These data indicate that chondrocyte autophagy is required for PC2 secretion from the ER.

The presence of PC2 in at least 15% of the AVs analyzed (FIG. 13c) and the colocalization of PC2 with the early markers of AV biogenesis ATG12 and ATG16L (FIG. 13d,e) shows that PC2 is an autophagy substrate in chondrocytes. Indeed, triple staining using Col2a1 and Sec31 antibodies in GFP-LC3 expressing chondrocytes showed that GFP-positive vesicles sequestered PC2 molecules in the ER (FIG. 6d).

Dual-color (mCherry-PC2 and GFP-LC3) live cell imaging experiments using Rx chondrocytes in which PC2 secretion was synchronized showed the selective sequestration of PC2 aggregates by GFP-LC3 positive vesicles (FIG. 6e,f).

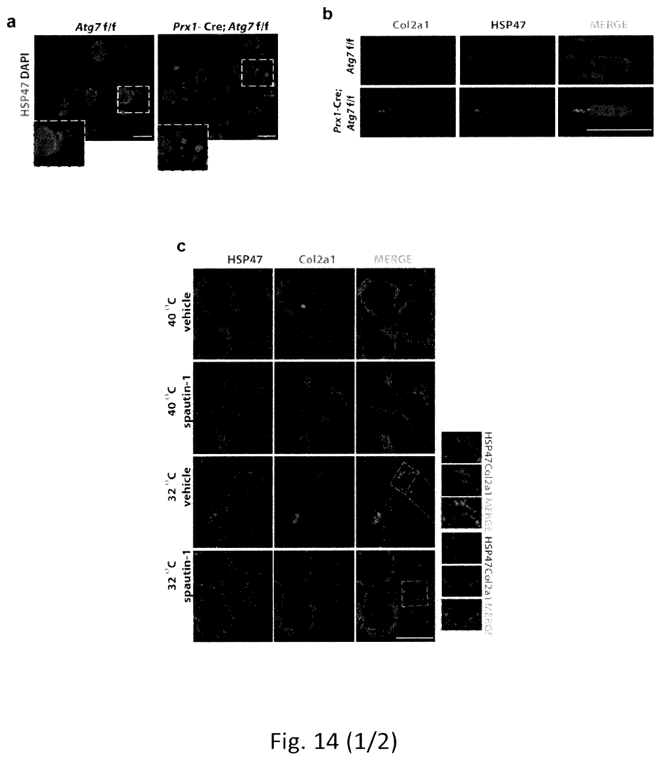

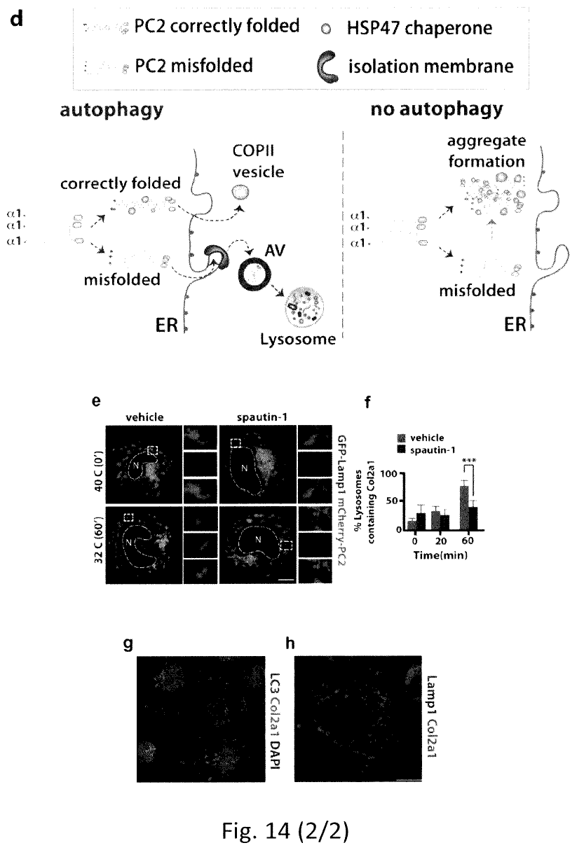

Furthermore, the 47 kDa collagen-specific chaperone HSP47, which associates to native PC2 triple helices in the ER and mediates their ER to cis-Golgi trafficking15, was excluded from the AVs containing PC2, suggesting that autophagy selectively recognizes non-native PC2 molecules in the ER (FIG. 13f). While in control chondrocytes HSP47 showed a diffuse distribution in Atg7Ff; Prx1-Cre growth plate chondrocytes it was clustered and colocalized with PC2 aggregates (FIG. 6g and FIG. 14a).

In addition, Spautin-1 treatment inhibited the ER to cis-Golgi trafficking of HSP47 in cultured chondrocytes (FIG. 14b). These data suggest that the accumulation of PC2 molecules in the ER may have detrimental consequences on the machinery involved in PC2 processing and secretion.

During autophagy, AVs target their cargo to lysosomes. Consistently dual-color (mCherry-PC2 and GFP-LAMP1) live cell imaging experiments showed progressive and autophagy-dependent accumulation of PC2 in GFP-LAMP1 vesicles (FIG. 6h and FIG. 14c).Total internal reflection fluorescence (TIRF) imaging failed to detect LC3 or LAMP1 positive vesicles fusing with the plasma membrane (PM). Furthermore, blocking the fusion of exocytic organelles with the PM using tannic acid (Newman T M et al. Eur J Cell Biol 1996; Medina D L et al, Dev Cell 2001) showed that none of the PC2-containing vesicles in the proximity of the PM were colabeled with LC3 or LAMP1 (FIG. 14d,e).

These data show that autophagy is required for PC2 homeostasis and secretion, rather than directly mediating PC2 exocytosis (FIG. 14f). This model also explains why autophagy is induced when chondrocytes boost PC2 production during early post-natal skeletal development (see FIG. 5a,b and 5d) and suggests that autophagy levels might be co-regulated with Colt production during bone development.

### Example 6—FGF18 Induces Autophagy in Growth Plate Chondrocytes

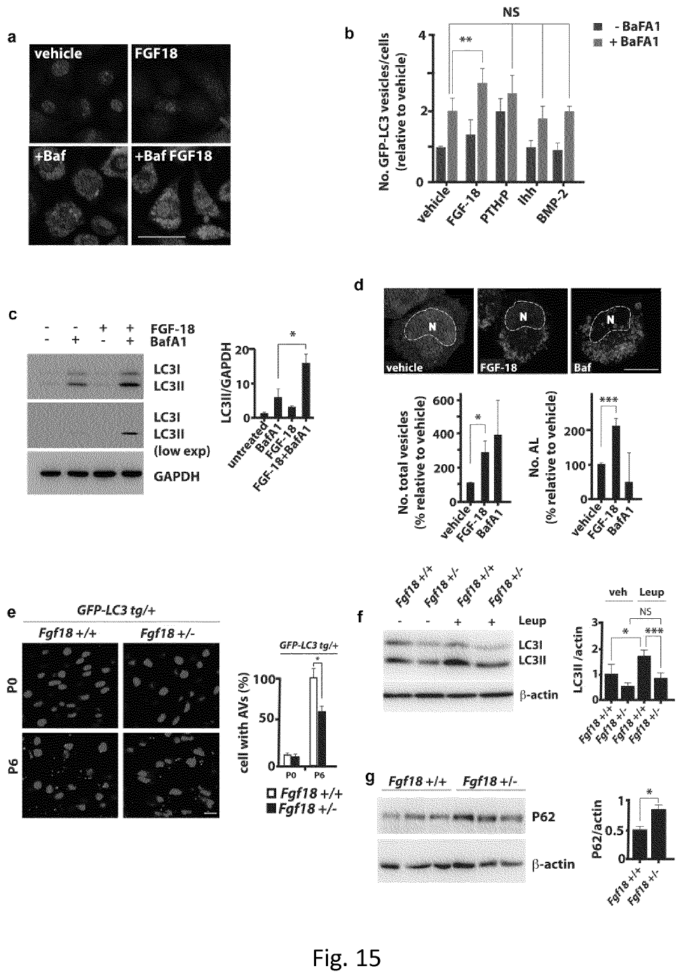

Primary chondrocytes isolated from GFP-LC3 mice were stimulated with FGF18 and other chondrogenic factors (Karsenty, G et al., Annu. Rev. Cell Dev. Biol. 2009) and autophagosome biogenesis was assessed in the presence of BafA1 (FIG. 15a). Among the factors tested, only FGF18 was able to increase significantly AV number (FIG. 15a,b). The effect of FGF18 on autophagy was confirmed by measuring LC3II levels in FGF18-treated wild type primary chondrocytes (FIG. 15c). FGF18 enhanced the autophagic flux, as demonstrated by an increased autolysosome number in Rx chondrocytes expressing the tandem fluorescent-tagged LC3 (mRFP-EGFP-LC3) protein (Kimura, S et al., Methods Enzymol. 2009) (FIG. 15d).

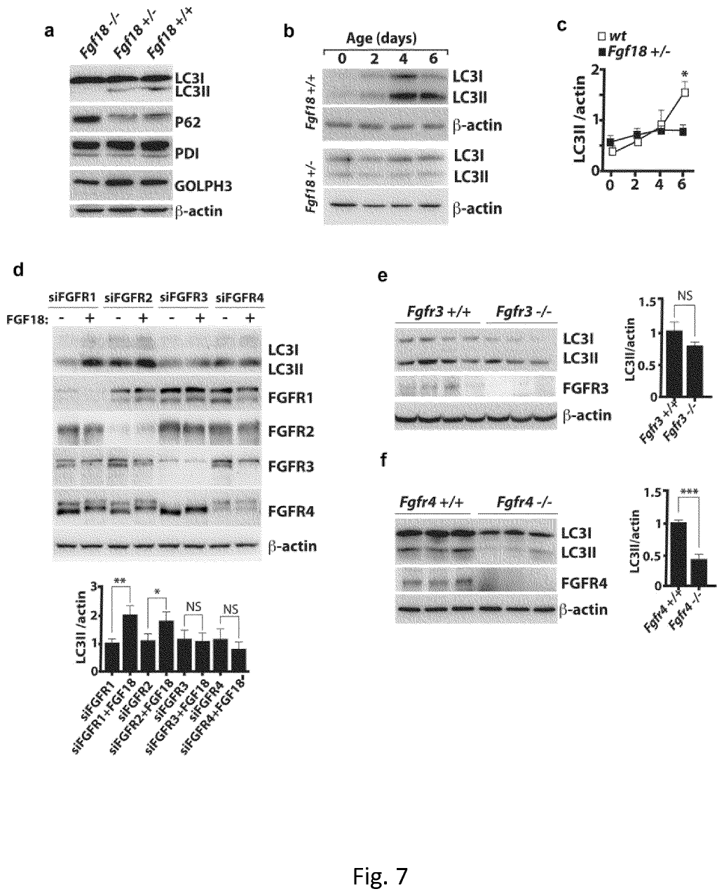

Most importantly, in vivo studies revealed that autophagy is completely inhibited in the growth plates of Fgfl 8−/− E18.5 embryos, as demonstrated by undetectable levels of LC3II and accumulation of the autophagy receptor P62/SQSTM1 compared to control mice (FIG. 7a). The levels of other organelle markers, such as PDI (ER) and GOLPH3 (Golgi) were not affected suggesting that lack of FGF18 specifically affects autophagy (FIG. 7a).

Fgfl 8−/− mice exhibit neonatal lethality (Liu Z et al. Genes Dev 2002), therefore the growth plates of Fgfl 8+/− mice, during early post-natal development, were analyzed: the levels of autophagy were similar in newborn Fgf18+/− and control mice, but the subsequent post-natal induction of autophagy was abrogated in Fgfl 8+/− mice (FIG. 7b,c).

Sections of growth plates isolated from P6 Fgfl 8+/−; GFP-LC3tg/+ mice had significantly fewer GFP-labeled AVs compared to sections isolated from control Fgfl 8+/+; GFP-LC3tg/+ mice (FIG. 15e).

Leupeptin treatment significantly increased LC3II levels in the growth plate of P6 control but not in Fgfl 8+/− mice suggesting reduced AV biogenesis in Fgfl 8+/− chondrocytes (FIG. 15f). Consistently, the level of P62/SQSTM1 was higher in Fgfl8+/− growth plates compared to controls at P30 (FIG. 15g). These data indicate that FGF18 is a critical regulator of chondrocyte autophagy during skeletal development.

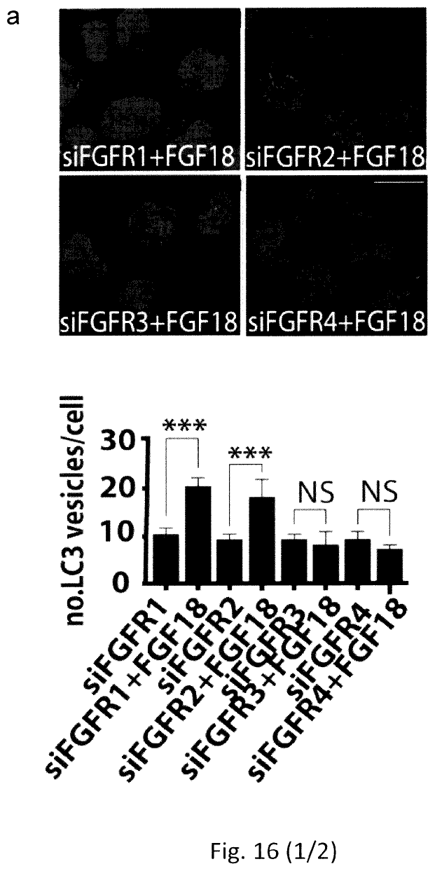

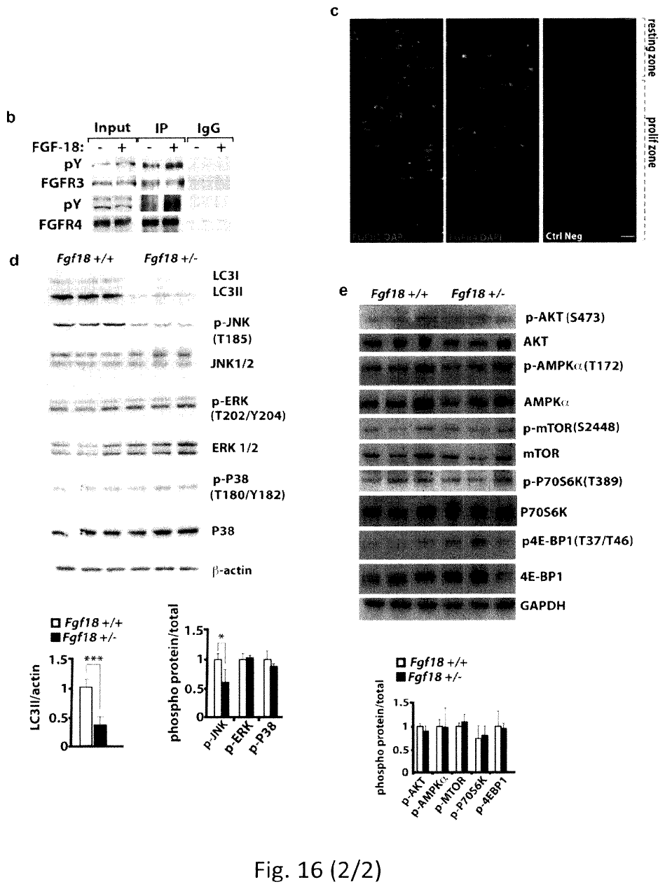

RNA interference of either Fgfr3 or Fgfr4, but not Fgfr1 and Fgfr2, inhibits FGF18-induced autophagy in Rx chondrocytes (FIG. 7d,e) In vivo, growth plate chondrocytes express both FGF receptor 3 and 4 (FIG. 16a), however, the levels of autophagy were significantly decreased only in the growth plates of Fgfr4−/− mice (FIG. 7f,g). These data indicate that the autophagy regulation by FGF18 is mediated by FGFR4.

### Example 7—Tat-Beclin 1 Peptide Normalized Autophagy Levels in the Growth Plates of Fgf18+/−

Canonical FGF signaling activates the mitogen-activated protein kinase (MAPK) pathway. The growth plates of Fgfl 8+/− mice show lower levels of JNK1/2 kinase activation than control mice (FIG. 16b). No changes were observed in the activation states of other members of the MAPK pathway (ERK and P38) or of other kinases involved in autophagy (FIG. 16c).

Active JNK1 phosphorylates Bcl2 and disrupts the Bcl2-Beclin 1 complex (Wei Y et al., Mol Cell 2008), leading to the activation of the Class III PI 3-kinase Vps34/Beclin 1 complex, which produces the phosphatidylinositol 3-phosphate (PI3P) required for AV biogenesis (Liang X H et al., Nature 1999).

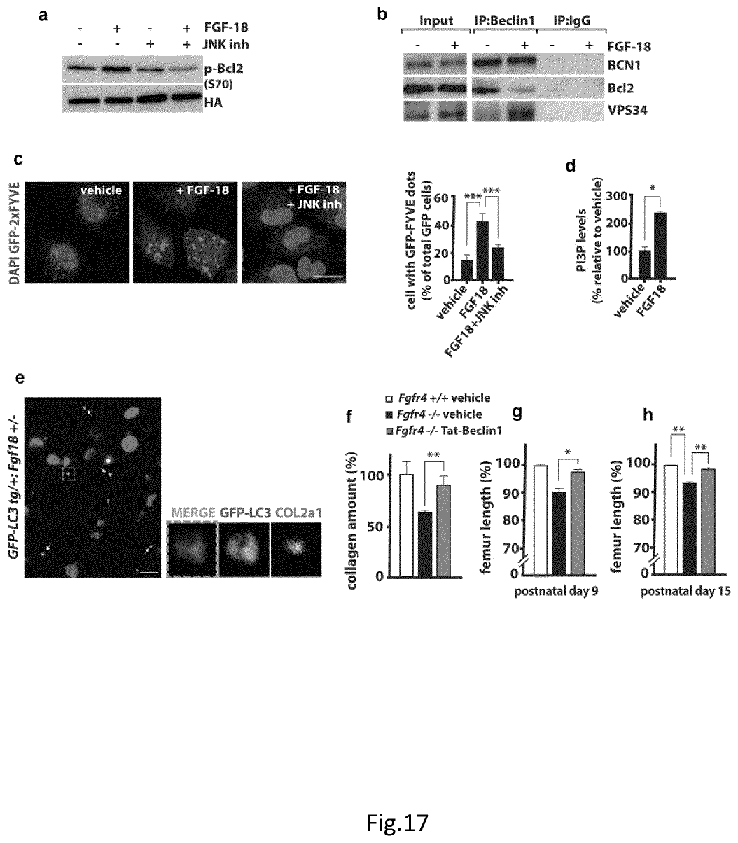

FGF18 increases the phosphorylation of Bcl-2 in a JNK-dependent manner (FIG. 17a); FGF18 stimulation decreased the interaction of Beclin 1-Bcl2 (FIG. 17b); FGF18 increases VPS34-Beclin 1 complex activity in a JNK dependent manner, as indicated by the amount of PI3P levels produced (FIG. 17c,d).

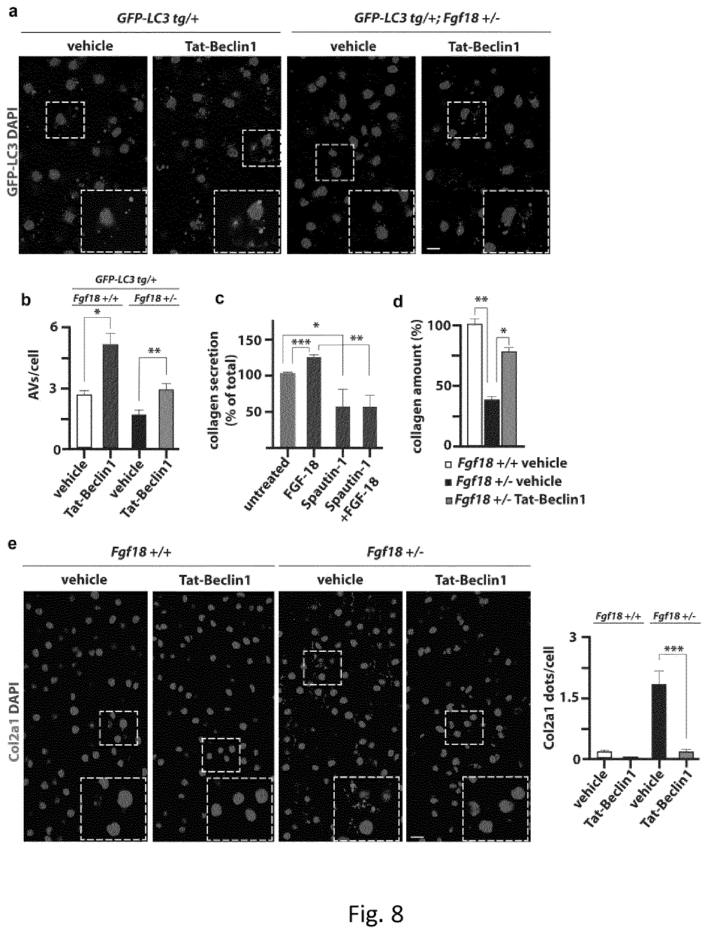

Enhancing Beclin 1 activity by intraperitoneal (IP) injection of a synthetic Tat-Beclin 1 peptide, as defined herein, normalizes autophagy levels in the growth plates of Fgf18+/−; GFP-LC3tg/+ mice (FIG. 8a, quantification in 8b). Thus, FGF18 induces autophagy through the regulation of Vps34/Beclin 1 complex activity.

Rx chondrocytes stimulated with FGF18 exhibited higher efficiency of PC2 secretion compared to non-stimulated cells, but addition of the autophagy inhibitor Spautin-1 hampered this increase (FIG. 8c). The Fgf18+/− growth plates were characterized by a severe reduction of collagen levels in the ECM (FIG. 8d) and by the presence of intracellular Col2a1 deposits in chondrocytes compared to control mice (FIG. 8e).

Therefore, the growth plate phenotype of Fgf18+/− mice mimics the one observed in mice lacking autophagy in chondrocytes. Notably, the few GFP-labeled AVs detectable in the growth plates of Fgfl 8+/−; GFP-LC3tg/+ mice contained PC2, further demonstrating that PC2 is an autophagy substrate in vivo (FIG. 17e).

Strikingly, Tat-Beclin 1 treatment restored Colt levels in the growth plates of Fgfl 8+/− mice (FIG. 8d) and completely eliminated the intracellular accumulation of PC2 in Fgfl 8+/− chondrocytes (FIG. 8e).

In addition, Tat-Beclin 1 treatment restored Colt levels and rescued femoral growth retardation in P9 Fgfr4−/− mice (FIG. 17f,g).

### Example 8—Vector for Expressing of Tat-Beclin 1 Peptide Tat-Beclin 1 AAV Vector Preparation

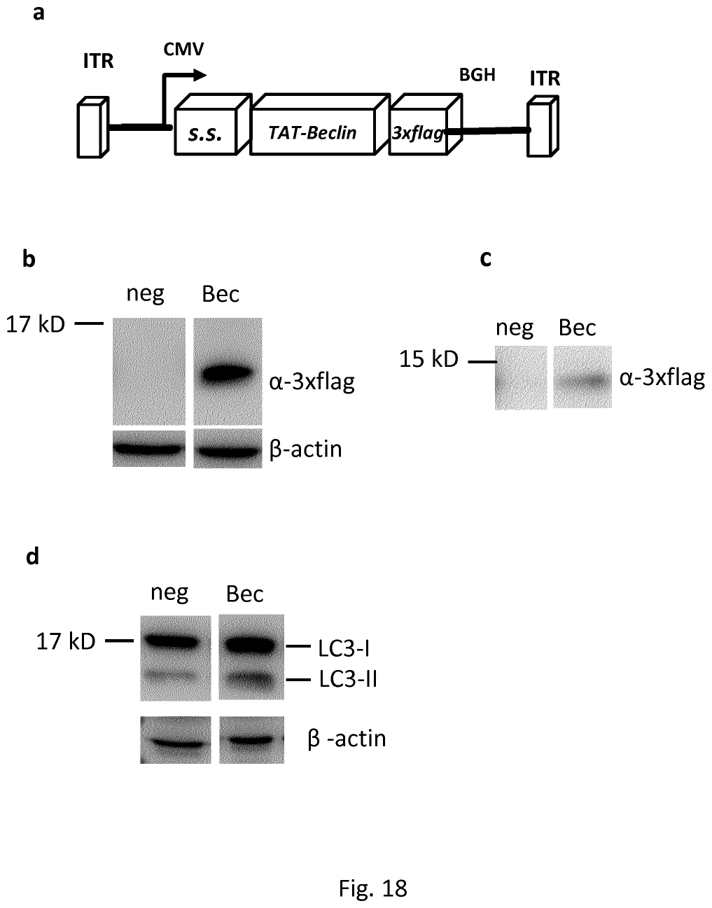

A vector for the expression of a Beclin 1 derivative peptide, according to preferred embodiments of the invention, was prepared by conventional means. The vector comprises a cassette having sequence SEQ ID NO:3, (FIG. 18a), according to a preferred embodiment of the invention. HEK 293 cells were transfected with said vector and harvested after 24h. The Beclin 1 derivative peptide was detectable in both cell lysate and conditioned media (FIGS. 18b and c) at 24 hours after transfection. LC3II increase was detectable in HEK 293 cell lysates from HEK293 cells incubated for 24h with Tat-Beclin 1 conditioned media. The vector of example 8 is suitable for been packaged into an adeno-associate virus (AAV) for viral delivery, according to a preferred embodiment of the invention.

### Materials and Methods of Examples 1-8

Animals: The Atg7 Fe and the GFP-LC3⁶ mouse lines were obtained from N. Mizushima (Tokyo Medical and Dental University Graduate School and Faculty of Medicine, Japan). The Prx-1 Cre line⁹ was purchased from Jackson Laboratories (strain n. 005584). Col2a1-Cre line was obtained from B. Lee (Baylor College of Medicine, Houston,). The fgfl 8²² and fgfr3²² KO line was a generous gift from D. Ornitz (Washington University, St. Louis). The fgfr4 was obtained from Dr. Seavitt (Baylor College of Medicine, Houston, Tex.). All mice used were maintained in a C57BL/6 strain background. Experiments were conducted in accordance with the guidelines of the Animal Care and Use Committee of Cardarelli Hospital in Naples and authorized by the Italian Ministry of Health.

The vector plasmid for Beclin 1 derivative peptide expression used in the examples was generated as follows: sFlt1-Tat-beclin1 sequence (sFlt1 is SEQ NO.6, Tat-Beclin 1 is SEQ No.7) was synthesized de novo and was cloned into a plasmid backbone which derived from the pAAV2.1 plasmid [Auricchio A, Hildinger M, O'Connor E, Gao G P, Wilson J M (2001)] Isolation of highly infectious and pure adeno-associated virus type 2 vectors with a single-step gravity-flow column. Hum Gene Ther 12: 71-76] and contained: the inverted terminal repeats (ITRs) of AAV serotype 2, the CMV promoter, the 3×flag tag, the WPRE and the BGH polyA. The vector plasmid was transfected into HEK293 cells using the calcium phosphate method. 24 hours later, Tat-Beclin 1 conditioned medium from transfected cells was harvested and added to a new plate of HEK293 cells. HEK293 cells were then incubated with conditioned media for 24 hours and finally harvested for Western Blot analysis.

Skeletal staining: Skeletons were fixed in 95% ethanol overnight (ON) and stained with alcian blue and alizarin red according to standardized protocols (http://empress.har.mrc.ac.uk/browser/). Three to five mice of each genotype were analyzed per stage. Measurement of bone length was performed using ImageJ software.

Tissue histology, immunohistochemistry and immunofluorescence: Histology was performed according to standardized procedures (http://empress.har.mrc.ac.uk/browser/). Briefly, femurs were fixed ON in 4% (wt/vol) paraformaldehyde (PFA) and then demineralized in 10% EDTA (pH 7.4) for 48h (demineralization was performed only if specimens were isolated from mice older than P5). Specimens were then dehydrated, embedded in paraffin and sectioned at 7 μm, and stained with hematoxylin and eosin. For BrdU staining mice were injected with 100 μL of 10 mM BrdU (Sigma) 4h before sacrifice. BrdU incorporation was detected using a Zymed BrdU staining kit (Invitrogen). The TdT-mediated dUTP Nick-End Labeling (TUNEL) assay was performed using the In situ Cell Death Detection kit (Roche). Counterstaining was performed using hematoxylin. For immunofluorescence, femurs were dissected from euthanized mice and fixed with buffered 4% PFA ON at 4° C., then washed with PBS and cryoprotected in successive sucrose solutions diluted with PBS (10% for 2 hours, 20% for several hours and 30% ON at 4° C.; all wt/vol), and finally embedded in OCT (Sakura). Cryostat sections were cut at 10 μm. Sections were blocked and permeabilized in 3% (wt/vol) BSA, 5% fetal bovine serum in PBS+0.3% Triton X-100 for 3 h and then incubated with the primary antibody ON. Sections were washed three times with 3% BSA in PBS+0.3% Triton X-100 and then incubated for 3 h with secondary antibodies conjugated with Alexa Fluor 488, or Alexa Fluor 568. The extracellular Col2a1 staining was performed by pretreating sections with chondroitinase ABC (Sigma) at 0.2 U/ml for 1 h at 37° C. prior to the blocking step. Intracellular Col2a1 staining was performed without chondroitinase ABC pretreatment to stain only the Col2a1 molecules that were not masked by proteoglycans. Primary antibodies used were: GFP, Lamp1 and HSP47 (Abeam), Col2a1 (1:30, Hybridoma Bank, II6B3), VapA, Sec31, Giantin, GM130, P115, Calreticulin were previously described 13. Nuclei were stained with DAPI and sections were mounted with vectashield (Vector laboratories). Images were captured using a Zeiss LSM700 confocal microscope. Colocalization analysis was performed calculating Mander's coefficient using ImageJ (colocalization analysis plug in).

Collagen Quantification and Analysis: Colorimetric assay was performed using the Sircol soluble collagen assay (Biocolor, UK) following the manufacturer's protocol. Briefly, femural and tibial cartilages were microdissected and collagen was acid pepsin extracted and complexed with Sircol dye. Absorbance was measured at 555 nm and concentration was calculated using a standard curve. Values were normalized to DNA levels calculated measuring the absorbance at 260 nm.

Electrophoretic analysis: Three femural cartilages were isolated from mice with the same genotype, pooled and homogenized in 0.5 ml of 1 mg/ml cold (4° C.) pepsin in 0.2 M NaCl, 0.5 M acetic acid to pH 2.1 with HCl and then digested at 4° C. for 24 hours, twice. The pellet was discarded and an equal volume (1 ml) of 4 M NaCl in 1 M acetic acid was added to precipitate collagen. The pellet was then resuspended in 0.8 ml of 0.2 M NaCl in 0.5 M acetic acid and was precipitated again three times. After the last precipitation the pellet was washed twice with 70% Et-OH in order to remove residual NaCl. The pellet was then dissolved in 0.8 ml 0.5 M acetic acid, and lyophilized. Subsequently it was resuspended in Laemmli buffer without Et-SH at a concentration of 2 mg/ml, denatured at 80° C. for 5 min and loaded on 6% SDS-PAGE. Gels were then stained with Coomassie Brilliant Blue R-250.

GAG quantification: GAG quantification was performed using the Blyscan sulfated glycosaminoglycan assay (Biocolor, UK) following the manufacturer's protocol. Briefly, femural and tibial cartilages were microdissected and GAGs were papain extracted at 65° C. ON and complexed with Blyscan dye. Absorbance was measured at 656 nm and concentration was calculated using standard curve. Values were normalized to DNA levels calculated measuring the absorbance at 260 nm.

Transmission electron microscopy: For EM analysis growth plates were fixed in 1% glutaraldehyde in 0.2M HEPES buffer. Small blocks of growth plates were then post-fixed in uranyl acetate and in OsO4. After dehydration through a graded series of ethanol, tissue samples were cleared in propylene oxide, embedded in Epoxy resin (Epon 812) and polymerized at 60° C. for 72h. From each sample, thin sections were cut with a Leica EM UC6 ultramicrotome and images were acquired using a FEI Tecnai-12 (FEI, Einhoven, The Netherlands) electron microscope equipped with Veletta CCD camera for digital image acquisition.

Tat-Beclin 1 peptide and Leupeptin treatment: Newborn mice were intraperitoneally injected daily with Tat-Beclin 1 peptide (Beclin 1 Activator II, retro-inverso Tat-Beclin 1, Millipore) at 20 mg/kg resuspended in PBS²⁵. Control mice were injected with vehicle only. Mice were sacrificed after 6 days (Col2a1 IF experiments) or 9 days (total collagen quantification). Leupeptin (Sigma Cat. L2884) was resuspended in water at 10 mM. Mice were give intraperitoneal injection at 40 mg/kg. Six hours after injection tissues were harvested and processed.

Tissue protein extracts for Western blotting: Femural and tibia cartilages were microdissected and lysed using a tissuelyser (Qiagen) in RIPA lysis buffer supplemented with 0.5% SDS, PhosSTOP and EDTA-free protease inhibitor tablets (Roche, Indianapolis, Ind., USA). Samples were incubated for 30 min on ice, briefly sonicated on ice and the soluble fraction was isolated by centrifugation at 14,000 rpm for 10 min at 4° C.

Chemicals: FGF18 (50 ng/ml), PTHrP (10 μg/ml), BMP2 (500 ng/ml) were from Peprotech, rhSHH (10 μg/ml) from R&D Systems. c-Jun N-Terminal kinase (INK) inhibitor (SP600125, Sigma-Aldrich, Milan, Italy) (50 μM) was used for the indicated time. Tannic Acid (Fluka chemika) was used at 0.5% final concentration in the medium for 1 h at 37° C. Bafilomycin A1 (Sigma) was used at 200 nM.

Cell Culture, transfections, SiRNA and Plasmids: Primary cultured chondrocytes were prepared from rib cartilage of P5 mice. Rib cages were first incubated in DMEM using 0.2% collagenase D (Roche) and after adherent connective tissue had been removed (1.5 h) the specimens were washed and incubated in fresh collagenase D solution for a further 4.5 h. Isolated chondrocytes were maintained in DMEM (Gibco) supplemented with 10% FCS and ascorbic acid (50 mg ml−1). Since an incomplete deletion of the Atg7 gene in Atg7f/f; Col2a1-Cre growth plates was observed (FIG. 9c) and the Prx1-Cre mice do not express Cre in chondrocostal chondrocytes (from where primary chondrocytes are routinely isolated), for the experiments measuring collagen secretion a chondrocyte line (Rx chondrocytes) in which autophagy was inhibited by Atg7RNAi and by pharmacological inhibition of Beclin 1 with Spautin-1 was employed. The Rx rat chondrosarcoma (RCS) chondrocyte cell line was previously described^{34,13}. Cells were transfected with Lipofectamine LTX and Plus reagent (Invitrogen) following a reverse transfection protocol. For SiRNA experiments, Si-genome smart pool (Dharmacon Thermo Scientific) were transfected to the final concentration of 50 nM. Cells were harvested 72 h after transfection. Plasmids: GFP-LC3 was a generous gift from Dr. Yoshimori (Osaka University), GFP-LAMP1 was from Dr. Fraldi (TIGEM institute) mCherry-PC2 was previously described13; Bcl2-HA was a generous gift from Dr. Renna (Cambridge), 2×FYYE-GFP was from Dr. Tooze (London Research Institute).

Live cell imaging: Rx chondrocytes were reverse transfected and plated in Mattek glass bottomed dishes. Collagen transport assays were performed by incubating cells at 40° C. on the heated stage for 2.5 h. Collagen release was initiated by lowering the temperature of the stage to 32° C. and medium being supplemented with 50 μg/ml ascorbate.

TIRF: Rx chondrocytes were reverse transfected and plated in Mattek glass bottomed dishes. Rx cells were synchronized on the heated stage for 2.5 h at 40° C. and released at 32° C., in medium supplemented with 50 μg/ml ascorbate in a humidified atmosphere with 5% CO2. The critical angle used was 65 degrees giving an evanescent field of 137 nm. Appropriate filter sets were used for GFP and mCherry detection. Frames were acquired on loop with no time delay (one frame roughly every 3s), for 15 min. All live cell imaging experiments was performed with a 60× Plan Apo oil immersion lens using a Nikon Eclipse Ti Spinning Disk microscope, and images and movies were annotated using the NIS Elements 4.20 software. Western blotting: Cells were washed twice with PBS and then scraped in lysis buffer (RIPA lysis buffer in the presence of PhosSTOP and EDTA-free protease inhibitor tablets—Roche, Indianapolis, Ind., USA). Cell lysates were incubated on ice for 20′, then the soluble fraction was isolated by centrifugation at 14,000 rpm for 10 min at 4° C. Total protein concentration in cellular extracts was measured using the colorimetric BCA protein assay kit (Pierce Chemical Co, Boston, Mass., USA). Protein extracts, separated by SDS-PAGE and transferred onto PVDF or nitrocellulose (for collagen) membranes, were probed with antibodies against P-JNK, JNK, P-Bcl-2, P-c-JUN (Cell Signaling Technology), HA, H3 Histone (Sigma-Aldrich, Milan, Italy) and LC3 (Novus Biologicals), p62 (BD Transduction Laboratories and Abnova), PDI (Cell Signaling), GOLPH3 (Abeam), p-ERK, ERK1/2 (Cell Signaling), p-P38, P38 (Cell Signaling), Beclin 1 (Cell Signaling), VPS34 (Sigma-Aldrich, Milan, Italy), b-actin (Novus Biologicals), GAPDH (Santa Cruz Biotecnology), Atg7 (Cell Signaling), p-mTORC1, mTORC1 (Cell Signaling), p-P70S6K, P70S6K (Cell Signaling), p-4EBP1, 4EBP1 (Cell Signaling), p-AKT, AKT (Cell Signaling), p-AMPKa, AMPKa (Santa Cruz Biotecnology), type II collagen (CIIC1b,Hybridoma Bank). Proteins of interest were detected with HRP-conjugated goat antimouse or anti-rabbit IgG antibody (1:2000, Vector Laboratories) and visualized with the Super Signal West Dura substrate (Thermo Scientific, Rockford, Ill.), according to the manufacturer's protocol. The Western blotting images were acquired using the Chemidoc-lt imaging system (UVP) and band intensity was calculated using imageJ software using “Gels and Plot lanes” plug-in.

High content screening analysis in GFP-LC3 primary chondrocytes: Primary chondrocytes were plated in CellCarrier-96 Black plates (6005558, Perkin Elmer). After identifying the nuclei with Hoechst 33342 (405 nm) staining, a cytoplasmic mask was drawn using Col2 staining (568 nm). To carry out the analysis the number of cytoplasmic GFP-LC3 spots in the cytoplasm of Col2 positive cells were counted, and expressed per cell. Levels of colocalization between GFP-LC3 and Col2a1 were assessed and expressed as %, using the parameters: area of colocalization of red spots with area of green spots normalized to total area of green spots. Image acquisition was performed using Opera High Content Screening System (PerkinElmer); image analysis was performed using Acapella High Content Imaging and Analysis Software (PerkinElmer). For GFP-LC3 puncta count, at least 1000 cells were analyzed for each treatment from 3 independent chondrocyte preparations. Repeated measures ANOVA was performed with TUKEYs post-hoc test. For GFP-LC3/col2a1 colocalization, at least 700 cells were analyzed per field from 2 different chondrocyte preparations.

Co-immunoprecipitation: Rx chondrocytes (100-mm dish) were grown in DMEM medium (Celbio, Milan, Italy) with 10% fetal bovine serum (FBS—Invitrogen corporation, Carlsbad, Calif., USA) and antibiotics. For FGF18 treatment, 70 to 80% confluent cells were cultured ON in DMEM with 10% adult bovine serum (Sigma-Aldrich, Milan, Italy) and then treated with FGF18 (50 ng/ml, 2 h) (Peprotech, Ottawa, Ontario) or DMSO vehicle. Rx chondrocytes were rinsed off the plate with ice-cold PBS, washed, and then scraped in IP lysis buffer (150 mM NaCl, 50 mM Tris-HCl pH 8.0, 1% NP-40, with one PhosSTOP and one EDTA-free protease inhibitor tablet per 10 ml—Roche, Indianapolis, Ind., USA). Cell lysates were rotated at 4° C. for at least 30 min, and then the soluble fraction was isolated by centrifugation at 14,000 rpm for 10 mM at 4° C. A fraction of the clarified lysate was used for Western blot analysis. Primary Beclin 1 (H-300) rabbit polyclonal (Santa Cruz Biotechnology, Santa Cruz, Calif.) antibody or rabbit pre-immune IgG were added to the lysates and rotated over night at 4° C., and then 25 μl of Protein A Sepharose beads (Sigma-Aldrich, Milan, Italy) were added and rotated for 2 h at 4° C. Immunoprecipitates were washed 3 times with cold lysis buffer. Whole cell lysates and immunoprecipitated proteins were boiled in 30 μl sample buffer, separated by SDS-PAGE on precast 4-15% gels (BioRad), transferred on PVDF membranes and probed with antibodies against Beclin 1 (Santa Cruz Biotechnology, Santa Cruz, Calif.), VPS34 (Sigma-Aldrich, Milan, Italy) and Bcl-2 (Cell Signaling Technology).

PI3K assay: PI3K activity in the Beclin 1 immunoprecipitates was determined using the PI3K ELISA kit (Echelon Biosciences, Inc., Salt Lake City, Utah) according to the manufacturer's instructions Immunocomplexes were incubated with a reaction mixture containing PtdIns(4,5)P2 substrate and ATP for 3 hours, and the amount of PtdIns(3,4,5)P3 generated from phosphatidylinositol 4,5-bisphosphate by PI3K was quantified using a competitive ELISA. Equal amounts of Beclin 1 immunoprecipitate were evaluated by Western blotting using Beclin 1 antibody.

Cell immunofluorescence: Chondrocytes were fixed for 10 min in 4% PFA in PBS and permeabilized for 30 min in 0.05% (w/v) saponin, 0.5% (w/v) BSA, 50 mM NH4Cl and 0.02% NaN3 in PBS (blocking buffer). The cells were incubated for 1 h with the primary antibodies, washed three times in PBS, incubated for 1 h with the secondary (Alexa fluor-labeled) antibody, washed three times in PBS, incubated for 20 min with 1 μg/ml Hoechst 33342 and finally mounted in Mowiol. All confocal experiments showing colocalization were acquired using slice thickness of 0.5 mm using the LSM 710 confocal microscope equipped with a 63×1.4 numerical aperture oil objective.

Procollagen secretion assay: To follow PC2 secretion in Rx chondrocytes, cells were pretreated ON with ascorbate (100 μg/ml) in DMEM without FCS. Cells were then labeled with 37.5 μCi/mL 2,3 3H-Proline (Perkin Elmer) for 4 h at 40° C. in the same medium then shifted to 32° C. in DMEM without FCS containing cold proline (10 mM), 20 mM HEPES pH 7.2 and ascorbate (100 μg/ml). After 0, 30 and 60 minutes the medium and cells were collected, lysed and proteins precipitated in saturated ammonium sulfate ON and resuspended in Laemmli buffer. Samples were run on 4-15% precast gels (Biorad), transferred onto nitrocellulose membrane (Whatman, Perkin Elmer) and developed by autoradiography using the BetaIMAGER-D system and analyzed using M3 Vision software (Biospace Lab).

### Example 9—Altered Autophagy in MPS VII Primary Chondrocytes

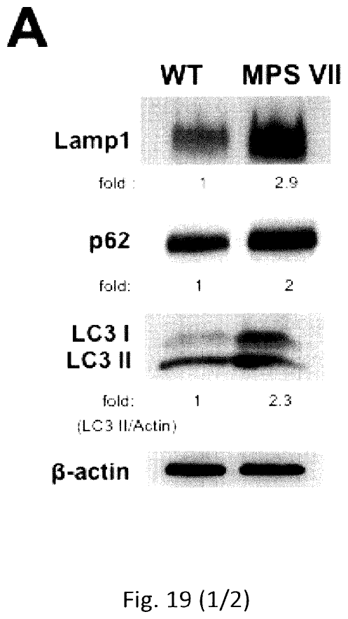

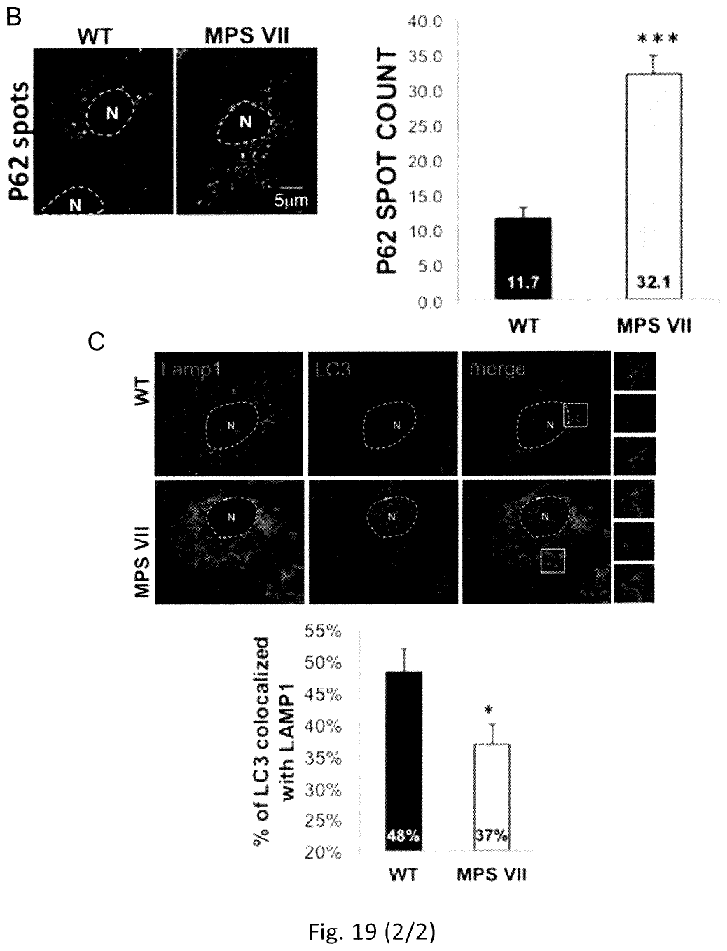

Primary chondrocytes were isolated from the rib cage of post-natal day 5 mice (wild-type and MPS VII) and were plated at the density of 10⁵ cells/cm². After 3 days in culture cells were splitted in 12 wells chamber for biochemical analysis (FIG. 19a) or on cover slips for immunofluorescence analysis (FIG. 19b, c). Accumulation of LAMP1 and of the lipidated LC3 (LC3II) markers of endolysosomes and of autophagosomes were detected by western blot analysis (FIG. 19a).

Primary chondrocytes isolated from chondrocostal cartilage of newborn MPS VII mice show prominent lysosomal storage phenotype characterized by cytoplasm filled with giant lysosomes, which were undetectable in chondrocytes isolated from control littermates (FIG. 19c). Without being bound to theory, accumulation of autophagosomes was most likely the consequence of impaired autophagosome maturation (e.g fusion with endolysosomes) rather than autophagy induction, as demonstrated by defective LAMP1-LC3 colocalization (FIG. 19c) and accumulation of autophagy substrate p62 (FIG. 19a). Double immune labeling of LAMP1 and LC3 showed that while in control chondrocytes LC3 showed 48% co-localization with LAMP1, in MPS VII chondrocytes this value did not exceed 37% (FIG. 19c), in addition immunofluorescence of the autophagy receptor p62 revealed MPS VII chondrocytes engulfed with a significant higher number of p62 puncta (FIG. 19b). MPS VII chondrocytes thus show a defective cargo delivery to lysosome by autophagosomes.

### Example 10—Altered mTORC1 Signaling in MPS VII Primary Chondrocytes

The mTORC1 kinase promotes anabolic processes, such as protein and lipid synthesis, in response to nutrients and growth factors stimulation⁵⁵. In addition, mTORC1 regulates lysosome/autophagy and proteasome functions through both transcriptional and post-translational mechanisms^{53,56}. Thus, mTORC1 controls the cellular balance between catabolic and anabolic metabolisms in response to nutrient levels.

Major regulators of mTORC1 are amino acids that can be either supplied with the diet or de-novo synthesized starting from metabolic intermediates⁵⁷. In addition the amino acid pool produced by lysosome and proteasome-mediated protein catabolism can also influence mTORC1 signaling⁵⁴. However, the physiological relevance of this source of amino acids as regulator of mTORC1 activity is still largely unknown.

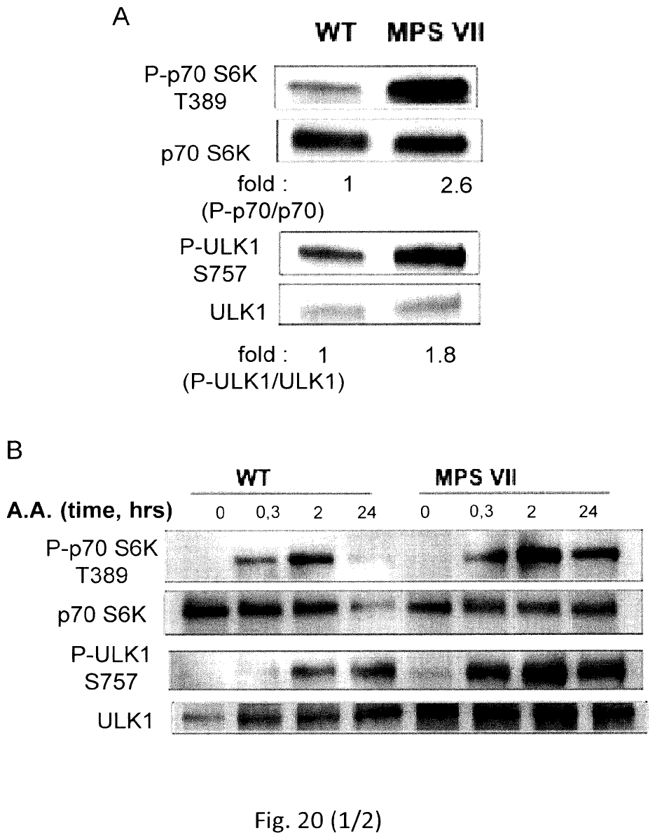

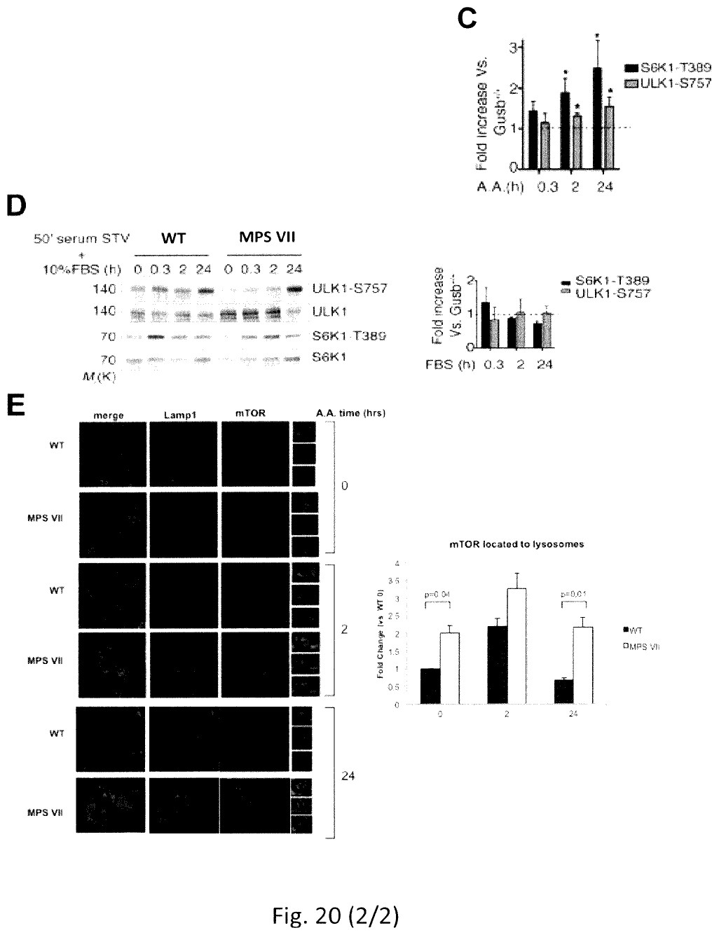

Primary chondrocytes were isolated from the rib cage of P5 mice (wild-type and MPS VII) and were plated at the density of 10⁵ cells/cm². After 3 days in culture cells were splitted in 12 wells chamber for biochemical analysis (FIG. 20a-d)).

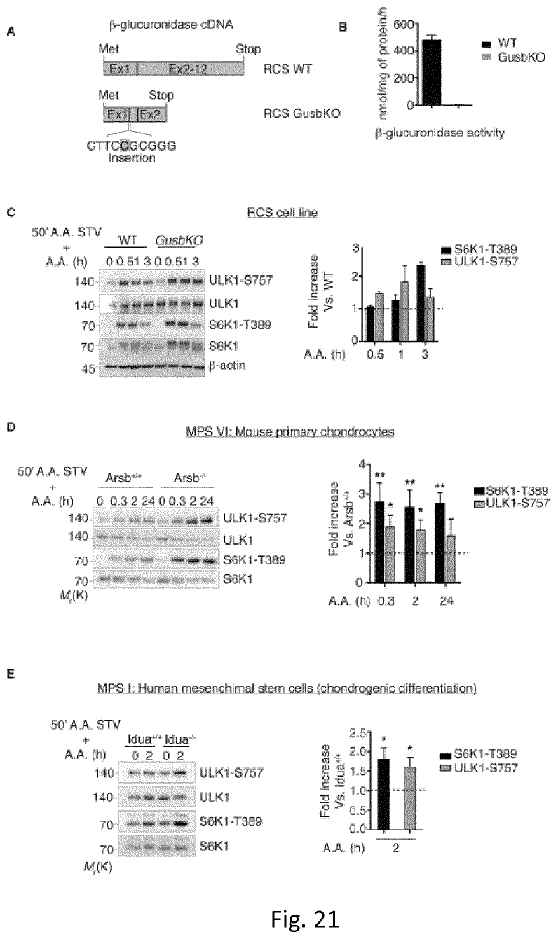

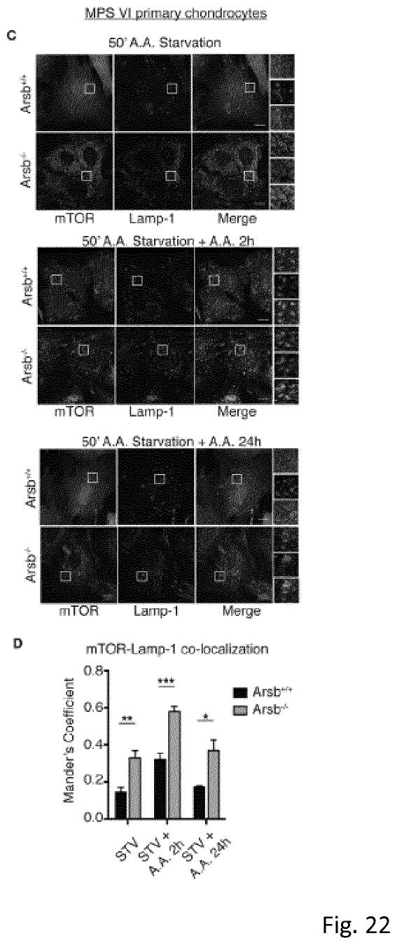

The activity of mTORC1 was analyzed in mouse primary chondrocytes and in a RCS chondrocytes isolated from mouse models of MPSVII (Gusb−/−)⁵⁸ and MPSVI (Arsb−/−)⁵⁹, mesenchymal-derived chondrocytes⁶⁰ isolated from three MPSI⁶¹ human patients and RCS model of MPSVII (GusbKO) generated by Crisp/Cas9 technology, all showed enhanced mTORC1 signaling (n enhanced phosphorylation of p70 S6 Kinase and of ULK1) in response to amino acid stimulation compared to their correspondent controls (FIG. 20a-c, FIG. 21 a-e). To gain insight into this observation experiments of starvation/refeeding of aminoacids (AA) and serum alone or in combination, being both potent mTORC1 activators, were performed. Cells were serum- or AA-starved for 1h and then treated for 0.3, 2 and 24 hours for each condition. No differences in p-P70S6K and p-ULK1 phosphorylation were observed upon serum stimulation alone (FIG. 20d), but stimulation with AA alone showed enhanced and more persistent phosphorylation of mTORC1 substrates in MPS VII chondrocytes compared to controls (FIG. 20b-c). MPS VII cells present upregulated mTORC1 signaling, compared to wt levels, throughout the experimental time-course (FIG. 20c) Amino acids are main mediator of mTORC1 association to lysosomes, a prerequisite for its activation. Co-localization experiments showed enhanced association of mTORC1 with lysosomes in both starved and nutrient stimulated MPSVII and MPS VI chondrocytes compared to control cells (FIG. 20e and FIG. 22, c-d, respectively).

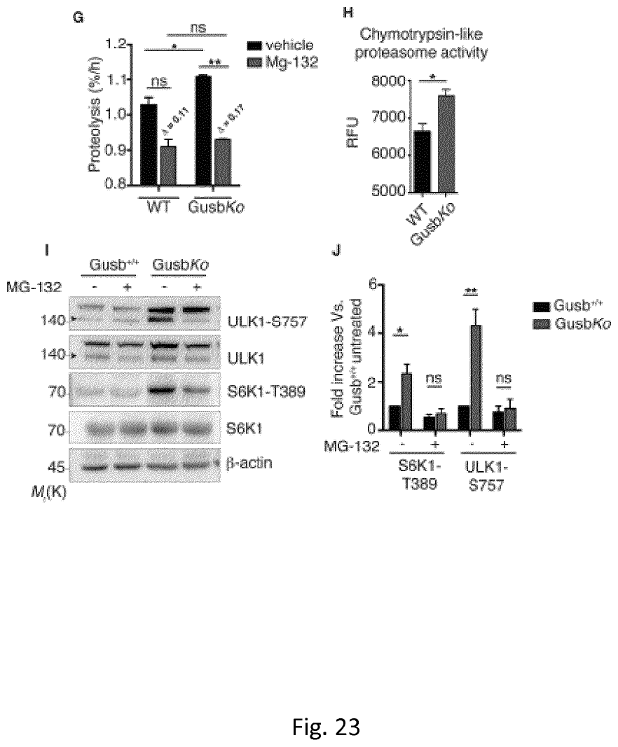

The response of MPS VII chondrocytes to growth factor (FBS 10%) stimulation was similar to that observed in control cells (FIG. 20D), suggesting that the sensing of amino acid by mTORC1 was impaired in MPS cells. Intracellular amino acids levels can also depend on rate of proteolysis. Despite having an impaired lysosome function, GusbKO chondrocytes had a higher protein degradation rate compared to control chondrocytes. Notably this increase can be completely blunted by the addition of the proteasome inhibitor Mg132, suggesting that it was largely due to proteasomal degradation (FIG. 23 g). Consistently, the proteasome activity was significantly higher in GusbKO compared to control chondrocytes (FIG. 23 h). An enhanced proteasome-mediated proteolysis can increase mTORC1 signaling (REF manning), and accordingly, Mg132 treatment normalized mTORC1 signaling in GusbKO chondrocytes (FIG. 23 i-j). These data suggest that the enhanced mTORC1 signaling in MPS chondrocytes may be caused, at least in part, by the amino acids generated by proteasome mediated proteolysis.

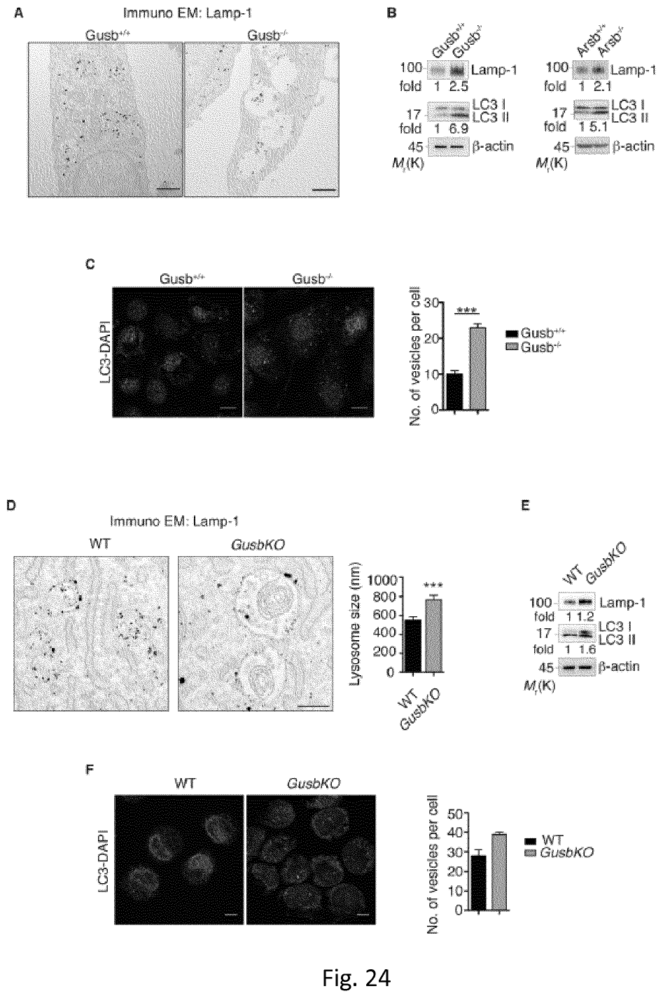

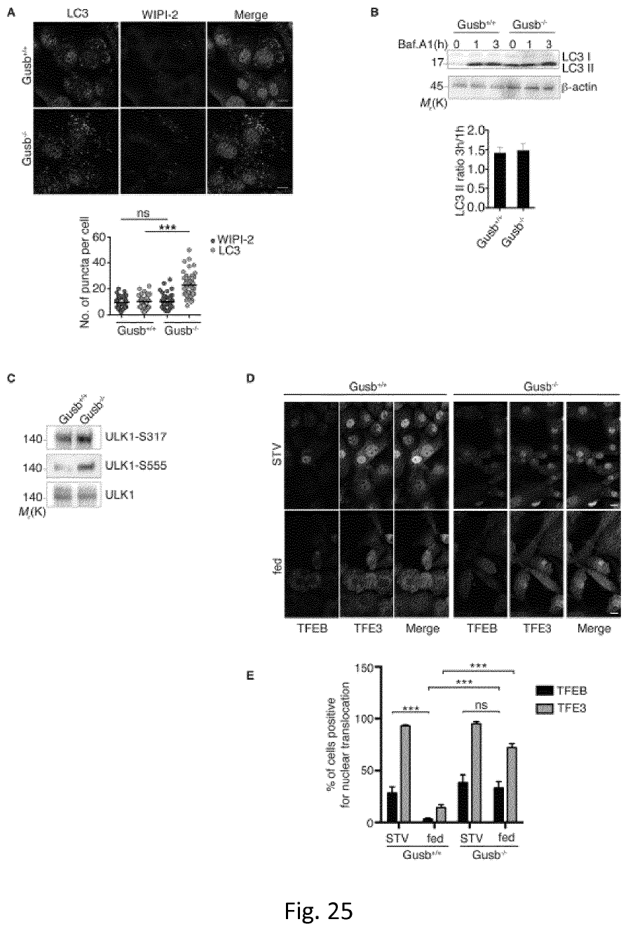

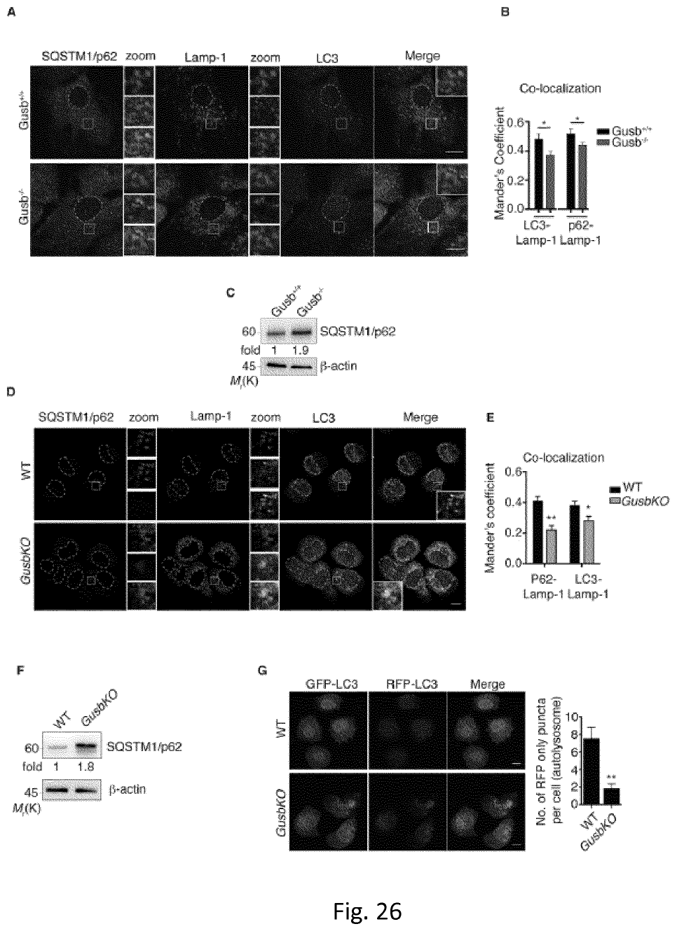

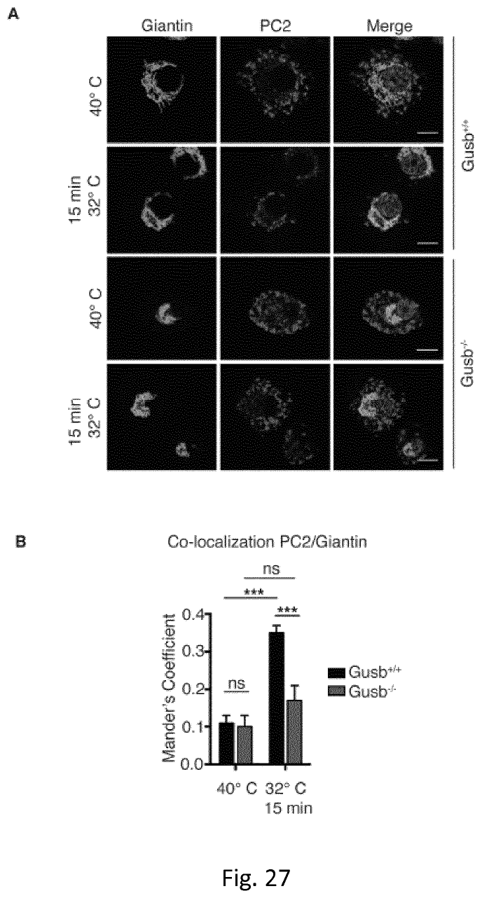

MPS chondrocytes showed a severe lysosome phenotype as demonstrated by enlarged Lys filled with undigested substrates and accumulation of the lysosomal marker LAMP1. In addition, the inventors also observed a significant accumulation of AVs, as demonstrated by increase number of LC3 positive vesicles and accumulation of the autophagosome-associated form of MAPLC3B protein (LC3II) (FIG. 24 a-h). Notably, despite the increase of mTORC1 activity, AV biogenesis was normal in MPSVII cells compared to controls as assessed by WIPI2 puncta formation and time course analysis of LC3-I to -II lipidation in presence of the lysosomal inhibitor Bafilomycin A1 (FIG. 25a-b). This result could be due to a compensatory activation of other, mTORC1 independent, autophagy pathways, such as phosphorylation of ULK1 by AMPK²¹ and increased TFEB/TFE3 nuclear localization, in MPS compared to control chondrocytes (see FIG. 25 c-e and Sardiello et al.⁶²). The accumulation of AV was rather the consequence of a defective AV digestion by Lys, as demonstrated by defective AV-Lys co-localization and accumulation of the P62/SQSTM1 autophagy substrate in MPS compared to control chondrocytes (FIG. 2 and FIG. 26 a-h). Consistent with an impaired autophagy, the inventors observed defective type II procollagen (PC2) trafficking in Gusb−/− chondrocytes compared to control cells (FIG. 27).

Without being bound to theory, enhanced activity of mTORC1 could be a consequence of increased association with lysosomes in MPS VII cells.

### Example 11—Pharmacological Inhibition of mTORC1 Restores Autophagy Flux in MPS VII Chondrocytes

Primary chondrocytes were isolated from the rib cage of P5 mice (wild-type and MPS VII) and were plated at the density of 10⁵ cells/cm². After 3 days in culture cells were splitted in 12 wells chamber, synchronized with AA, treated with Torin1 (1 μM) for 24 hours and harvested for biochemical analysis.

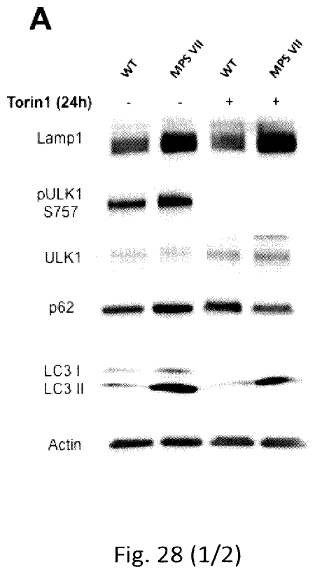

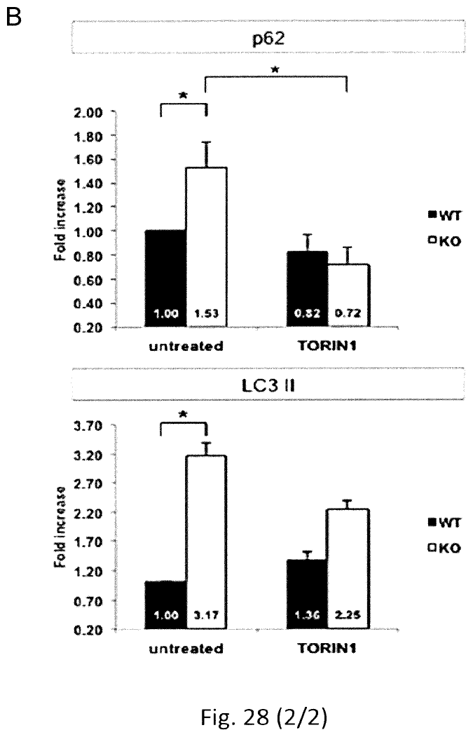

Pharmacological inhibition of mTORC1 with Torin1 completely suppresses phosphorylation of mTORC1 substrates and rescues the autophagy defects in MPSVII chondrocytes, as demonstrated by normalization of LC3 II and p62 levels (FIG. 28a-b).

These data indicate that normalization of mTORC1 signaling is sufficient to ameliorate the cellular phenotype in MPS VII chondrocytes, indicating that mTORC1 dysfunction could account, at least in part, for the autophagy defects in MPS VII chondrocytes.

### Example 12—Genetic Limitation of mTORC1 in MPS VII Chondrocytes Rescues Both mTORC1 Altered Signaling and Autophagy Flux

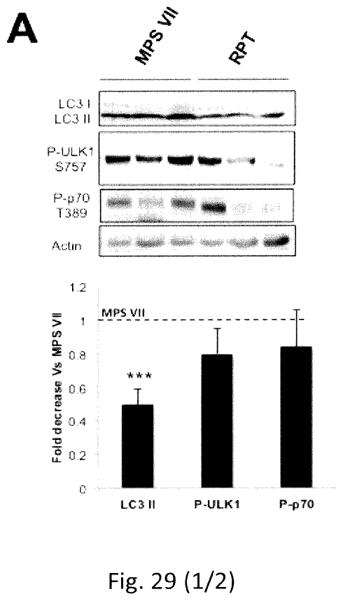

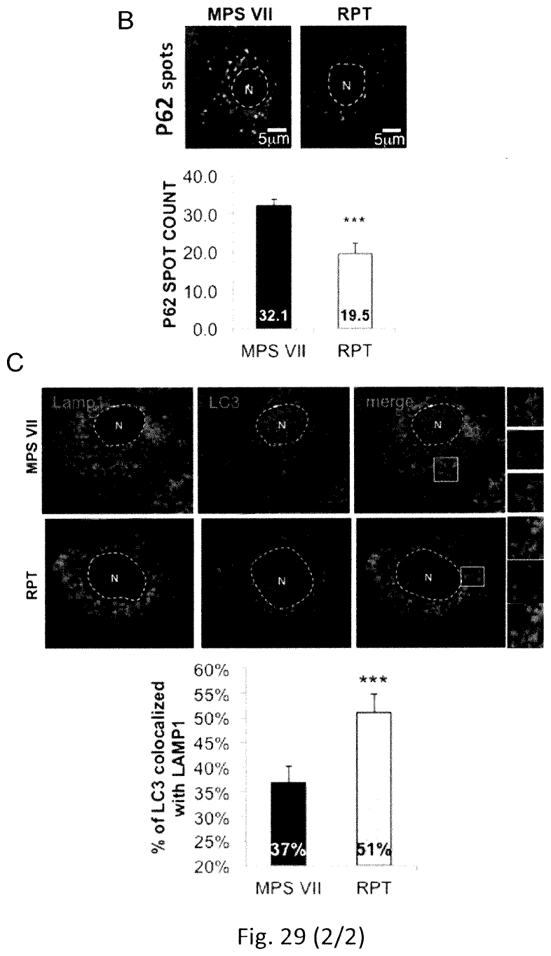

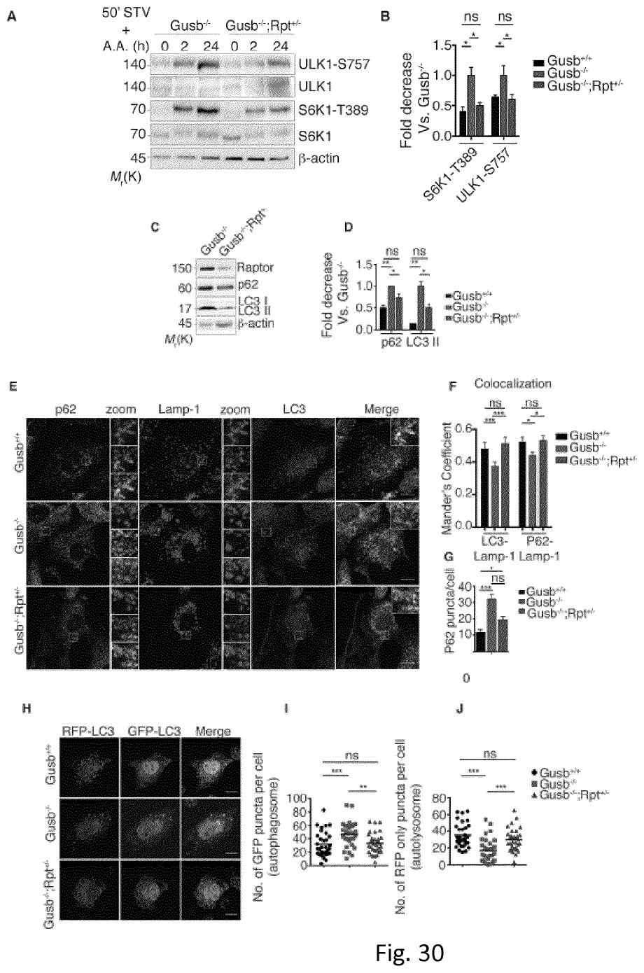

Raptor (RPT or Gusb−/−; Rpt+/−) mice are MPS VII mice (Gusb−/−) carrying only one functional copy of raptor allele (FIG. 29 and FIG. 30). Primary chondrocytes were isolated from the rib cage of P5 mice (MPS VII and RPT) and were plated at the density of 10⁵ cells/cm². After 3 days in culture cells were splitted in 12 wells chamber for biochemical or immunofluorescence analysis.

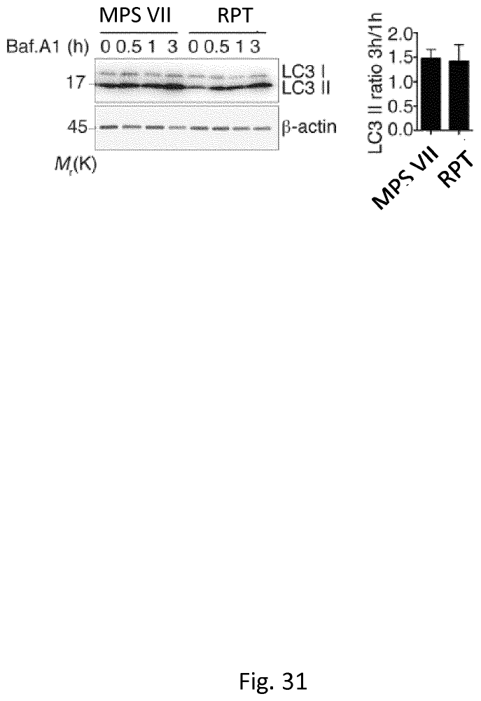

Genetic limitation of mTORC1 rescues the altered signaling found in MPSVII chondrocytes, thus RPT cells show 20% reduction in the levels of both P-ULK1 and P-p70S6K activation (FIG. 29a). This, in turn, is sufficient to ameliorate the autophagy defects as demonstrated by significant reduction of p62 puncta (FIG. 29b), reduction of LC3 accumulation (FIG. 29a), normalized autophagosome-lysosome fusion and cargo delivery to lysosomes (FIG. 29c).RPT primary chondrocytes (Gusb−/−; Rpt+/−) thus showed reduced accumulation of LC3II and of P62/SQSTM1 compared to MPS VII (Gusb−/−) chondrocytes (FIG. 30 c-j). This phenotype was most likely the consequence of a restoration of the autophagy flux, as demonstrated by enhanced AV-Lys co-localization and increased P62/SQSTM1 delivery to lysosomes in RPT compared to MPSVII chondrocytes. Notably, restoring mTORC1 signaling to normal levels did not alter AV biogenesis suggesting that an enhanced mTORC1 signaling directly impact the rate of AV-Lys fusion (FIG. 31).

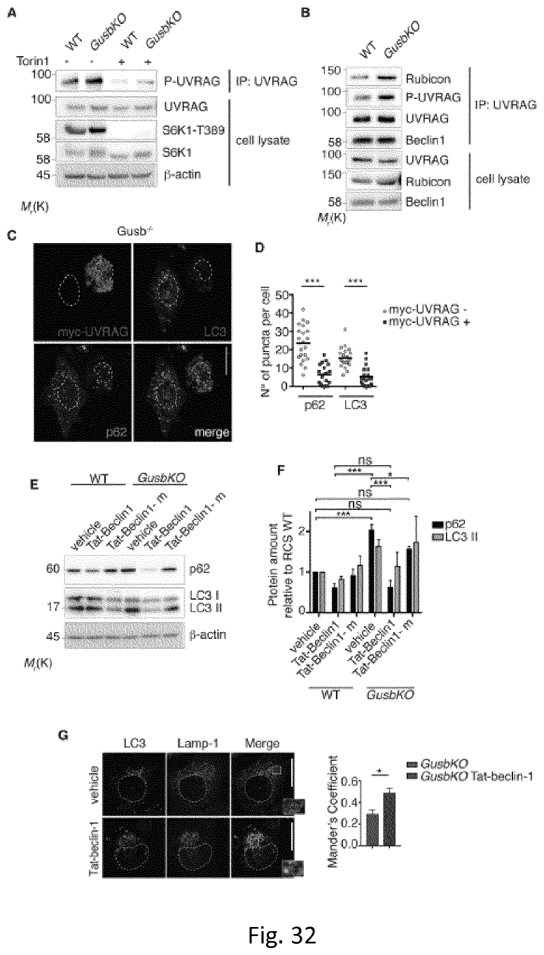

mTORC1 can inhibit AV-Lys fusion by phosphorylation of UV radiation resistance-associated gene (UVRAG) protein, enhancing its affinity for the inhibitor partner Rubicon. Several lines of evidence suggested that this was the case in MPS chondrocytes: GusbKO cells had higher levels of UVRAG serine 497 (S497) phosphorylation compared to control cells, and this phosphorylation was blunted by the mTOR inhibitor Torin-1 (FIG. 32 A); the interaction of UVRAG with Rubicon was higher in GusbKO compared to control cells (FIG. 32 b); forced overexpression of UVRAG rescued AV and P62/SQSTM1 accumulation in Gusb−/− cells (FIG. 32 c). These data suggest that mTORC1 inhibits AV maturation in MPS chondrocytes at least in part by inhibiting UVRAG activity. Notably, inventors obtained similar results by treating GusbKO cells with TAT-Beclin1 peptide. This peptide enhances the activity of Beclin1 protein that forms, together with UVRAG, VPS34 and VPS15, the ClassIII-VPS34 complex II involved in endolysosome maturation and AV-Lys fusion²⁵⁶³ (FIG. 32 d-f).

### Example 13—Limitation of mTORC1 Signaling as a Therapeutic Approach for the Treatment of Bone Growth Retardation in MPS VII Mice

WT, MPS VII and RPT littermates were sacrificed at post-natal day 15. Four hours before sacrifice mice were injected with BrdU at 0.1 mg/g body weight. Skeletons were prepared and stained with Alizarin Red/Alcian Blue. For analyses on sections limbs were collected, decalcified, processed and sectioned in paraffin.

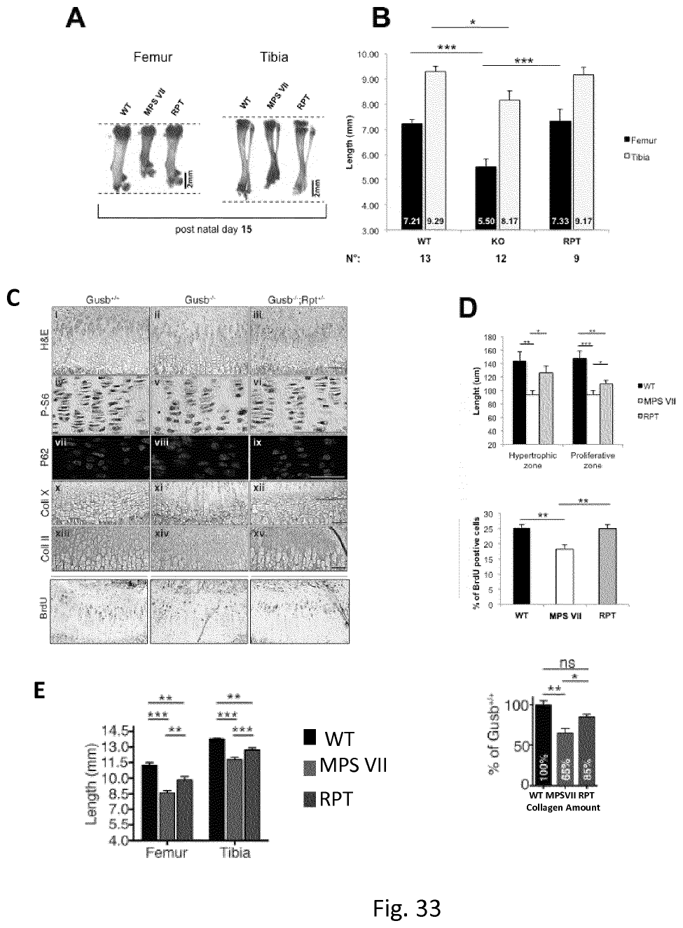

Skeletal preparations showed that removing one allele of raptor rescued MPS VII mice short stature at post-natal day 15, as determined by femur and tibia length (A). Importantly this rescue is maintained up to post-natal day 30 (FIG. 33e).

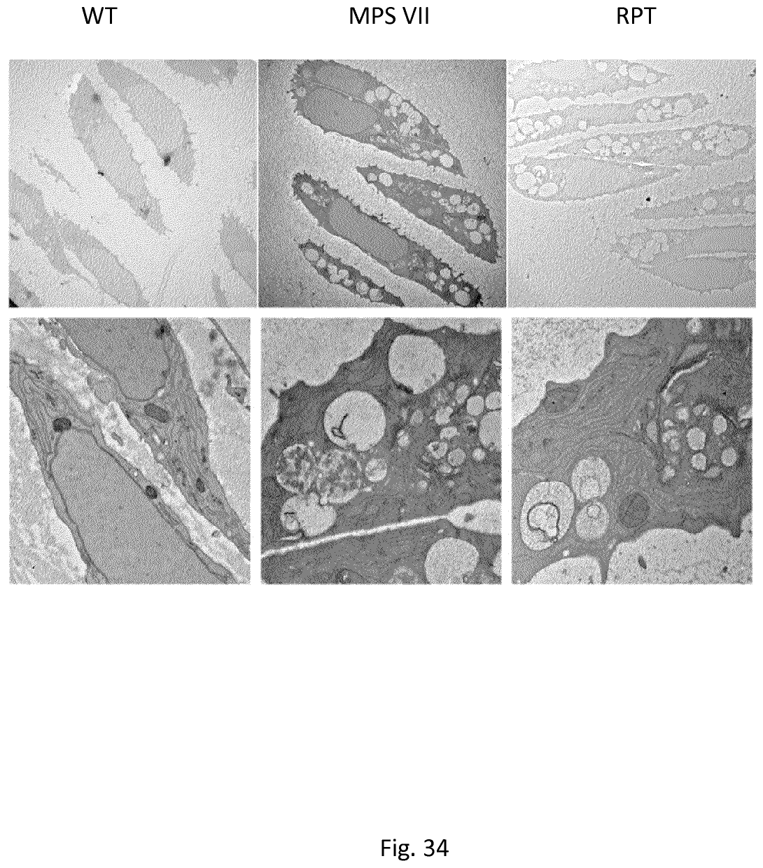

Consistently histological analysis of femur and tibia sections showed that chondrocyte proliferation measured by BrdU incorporation, which was significantly reduced by 7% in the P15 MPS VII, was indistinguishable from wild-type in RPT P15 mice (FIG. 33 c-d lower panel). As a result, the zones of hypertrophic and proliferative chondrocytes were larger as demonstrated by Haematoxylin/Eosin (H&E) and Collagen type X immunostaining (FIG. 33b-c). Limitation of mTORC1 signaling in vivo thus reduced S6 phosphorylation, p62/SQSTM1 levels and significantly improved collagen levels in the growth plates of RPT compared to MPS VII mice, even without reverting chondrocyte lysosomal storage (FIG. 34).

### Materials and Methods Examples 9-13

Skeletal staining: Skeletons were fixed in 95% ethanol overnight (ON) and stained with alcian blue and alizarin red according to standardized protocols (http://empress.har.mrc.ac.uk/browser/). Three to five mice of each genotype were analyzed per stage. Measurement of bone length was performed using ImageJ software.

Tissues and histology: Histology was performed according to standardized procedures (http://empress.har.mrc.ac.uk/browser/). Briefly, femurs were fixed ON in 4% (wt/vol) paraformaldehyde (PFA) and then demineralized in 10% EDTA (pH 7.4) for 48h. Specimens were then dehydrated, embedded in paraffin and sectioned at 7 μm, and stained with hematoxylin and eosin. For BrDU staining mice were injected with 200 μl of 10 mM BrDU (Sigma) 4h before sacrifice. BrDU incorporation was detected using a Zymed BrDU staining kit (Invitrogen). Counterstaining was performed using hematoxylin Immunohistochemistry were performed according to standardized protocols. Briefly, type X collagen (Hybridoma bank) staining were performed pretreating paraffin-embedded sections with 1 mg/ml pepsin in 0.1 M Acetic Acid, 0.5 M NaCl for 2 h at 37° C., and then treated with 2 mg/ml hyaluronidase in 0.1 M TBS for 1 h at 37° C., prior to the blocking step. Endogenous peroxidases were quenched with 3% hydrogen peroxide, sections were then incubated with blocking serum and primary antibody over night at 4° C. Signals were developed using Vectastain Elite ABC kit (Vector Laboratories) and NovaRED Peroxidase Substrate kit (Vector Laboratories).

Cell Culture: Primary chondrocytes were isolated from the rib cage of post-natal day 5 mice. Rib cages were first incubated in DMEM using 0.2% collagenase D (Roche) and after adherent connective tissue had been removed (1.5 h) the specimens were washed and incubated in fresh collagenase D solution for a further 4.5 h. Isolated chondrocytes were maintained in DMEM (Gibco) supplemented with 10% FCS and were plated at the density of 105 cells/cm2. After 3 days in culture cells were splitted in 12 wells chamber for biochemical analysis (Western blot) or on cover slips for immunofluorescence analysis. For amino acid stimulation cells were starved 1h in RPMI-1640 medium (USbio) without amino acids and supplemented with 10% dialyzed FBS (Invitrogen, Life Technologies) then cells were treated for the indicated time-points with a mixture of essential amino acids, non-essential amino acids and L-glutammine (Invitrogen, Life technologies) at the final concentration of 3×.

Western blotting: Cells were washed twice with PBS and then scraped in lysis buffer (RIPA lysis buffer in the presence of PhosSTOP and EDTA-free protease inhibitor tablets—Roche, Indianapolis, Ind., USA). Cell lysates were incubated on ice for 20′, then the soluble fraction was isolated by centrifugation at 14,000 rpm for 10 min at 4° C. Total protein concentration in cellular extracts was measured using the colorimetric BCA protein assay kit (Pierce Chemical Co, Boston, Mass., USA). Protein extracts, separated by SDS-PAGE and transferred onto PVDF or nitrocellulose (for collagen) membranes, were probed with antibodies against P-ULK(5757), ULK1, P-p70S6K (T389), p70S6K (Cell Signaling Technology), LC3 (Novus Biologicals), p62 (BD Transduction Laboratories and Abnova), b-actin (Novus Biologicals), LAMP1 (Abacam). Proteins of interest were detected with HRP-conjugated goat antimouse or anti-rabbit IgG antibody (1:2000, Vector Laboratories) and visualized with the Super Signal West Dura substrate (Thermo Scientific, Rockford, Ill.), according to the manufacturer's protocol. The Western blotting images were acquired using the Chemidoc-lt imaging system (UVP) and band intensity was calculated using imageJ software using “Gels and Plot lanes” plug-in.

Cell immunofluorescence: Chondrocytes were fixed for 10 min in 4% PFA in PBS and permeabilized for 30 min in 0.05% (w/v) saponin, 0.5% (w/v) BSA, 50 mM NH4C1 and 0.02% NaN3 in PBS (blocking buffer). The cells were incubated for 1 h with the primary antibodies, washed three times in PBS, incubated for 1 h with the secondary (Alexa fluor-labeled) antibody, washed three times in PBS, incubated for 20 min with 1 μg/ml Hoechst 33342 and finally mounted in Mowiol. All confocal experiments showing colocalization were acquired using slice thickness of 0.5 mm using the LSM 710 confocal microscope equipped with a 63×1.4 numerical aperture oil objective. Colocalization was measured using imageJ software using “JACoP” plug-in.

Results are given as means±standard errors of the means. Statistical analyses is performed using an unpaired, two-tailed Student t test. For all experiments significance is be indicated as follows: *, P≤0.05; **, P≤0.01; ***, P≤0.001.

The data provided by the inventors show a previously unanticipated role of chondrocyte autophagy in bone growth. Without being bound to theory, during early post-natal skeletogenesis, FGF18-FGFR4 complex induces the activation of INK kinase, which phosphorylates Bcl2 leading to the disruption of the Bcl2-Beclin 1 interaction and to the activation of the Beclin 1/Vps34 complex. This process leads to the production of a pool of PI3P required for autophagosome (AV) formation in chondrocytes. The induction of autophagy maintains PC2 homeostasis and prevents accumulation of PC2 in the ER during phases of high PC2 secretion. Chondrocyte autophagy appears to be dispensable when low levels of PC2 secretion are needed (e.g. pre-natal bone growth).Chondrocyte autophagy maintains the balance between synthesis, folding and secretion of PC2 in the ER during bone growth. This role is particularly important when PC2 synthesis is increased and massive secretion is needed to satisfy the high demand during post-natal bone growth. In these conditions a fraction of newly synthesized PC2 is degraded through autophagy probably due to imperfect folding or assembly.

Without being bound to theory, FGFR4 may regulate bone growth, at least in part; this occurs through modulation of autophagy.

Disruption of autophagy may lead to reduced femoral and tibial length (mainly post-natal role) and to deficient Col2 deposition in ECM (post-natal role); defective FGF signaling leads to defects in Col2 deposition in the ECM. Further pathogenetic mechanism can occur, leading to defects in the bone growth.

The inventors have demonstrated for the first time that activation of Beclin 1/Vps34 complex is beneficial in pathologies associated with bones developmental dysfunction, in particular long bones. Molecules according to the present invention are moreover capable of rescuing Col2 deposition defects and bone growth defects associated with bone growth disorders.

### REFERENCES

1. Karsenty, G. & Wagner, E. F. Dev. Cell 2, 389-406 (2002).
2. Shapiro, I. M., et al., Autophagy 10, 7-19 (2014).
3. Mizushima, N. & Komatsu, M. Cell 147, 728-741 (2011).
4. Wilsman, N. J., et al., J. Orthop. Res. Off Publ. Orthop. Res. Soc. 14, 927-936 (1996).
5. Kronenberg, H. M. Developmental regulation of the growth plate. Nature 423, 332-336 (2003).
6. Mizushima, N., et al Mol. Biol. Cell 15, 1101-1111 (2004).
7. Kabeya, Y. et al. EMBO J. 19, 5720-5728 (2000).
8. Komatsu, M. et al. J. Cell Biol. 169, 425-434 (2005).
9. Logan, M. et al. Genes. N. Y. N 2000 33, 77-80 (2002).
10. Ovchinnikov, D. A. , Genes. N. Y. N 2000 26, 145-146 (2000).
11. Klionsky, D. J. et al. Autophagy 8, 445-544 (2012).
12. Olsen, B. R. , Reginato, A. M. & Wang, W. Annu. Rev. Cell Dev. Biol. 16, 191-220 (2000).
13. Venditti, R. et al. Science 337, 1668-1672 (2012).
14. Liu, J. et al. Cell 147, 223-234 (2011).
15. Ishida, Y. & Nagata, K. Methods Enzymol. 499, 167-182 (2011).
16. Newman, T. M. , Tian, M & Gomperts, B. D. Eur. J. Cell Biol. 70, 209-220 (1996).
17. Medina, D. L. et al. Dev. Cell 21, 421-430 (2011).
18. Moore, E. E. et al. Osteoarthr. Cartil. OARS Osteoarthr. Res. Soc. 13, 623-631 (2005).
19. Karsenty, G. , Kronenberg, H. M. & Settembre, C. Annu. Rev. Cell Dev. Biol. 25, 629-648 (2009).
20. Kimura, S. , et al. , Methods Enzymol. 452, 1-12 (2009).
22. Liu, Z. , Xu, J. , Colvin, J. S. & Ornitz, D. M. Genes Dev. 16, 859-869 (2002).
22. Wei, Y. , Pattingre, S. , Sinha, S. , Bassik, M. & Levine, B. Mol. Cell 30, 678-688 (2008).
23. Liang, X. H. et al. Nature 402, 672-676 (1999).
24. Pattni, K. , Jepson, M. , Stenmark, H. & Banting, G. Curr. Biol. CB 11, 1636-1642 (2001).
25. Shoji-Kawata, S. et al. Nature 494, 201-206 (2013).
26. Lotz, M. K. & Carames, B. Nat. Rev. Rheumatol. 7, 579-587 (2011).
27. Srinivas, V. , Bohensky, J. , Zahm, A. M. & Shapiro, I. M. Cell Cycle 8, 391-393 (2009).
28. Ishida, Y. et al. Mol. Biol. Cell 20, 2744-2754 (2009).
29. Teckman, J. H. & Perlmutter, D. H. Am. J. Physiol. Gastrointest. Liver Physiol. 279, G961-974 (2000).
30. Houck, S. A. et al. Mol. Cell 54, 166-179 (2014).
31. Ornitz, D. M. & Marie, P. J. Genes Dev. 16, 1446-1465 (2002).
32. Settembre, C. et al. Genes Dev. 22, 2645-2650 (2008).
33. Lango Allen, H. et al. Nature 467, 832-838 (2010).
34. King, K. B. & Kimura, J. H. J. Cell. Biochem. 89, 992-1004 (2003).
35. Cho, J. Y. , et al. , Proc. Nat. Acad. Sci. 101: 609-614 (2004).
36. Nicole C. McKnight, et al. , PLOS Genetics Vol. 10(10) 2014
37. Levine B et al., Trends in Cell Biology, Vol 25(9) (2015)
38. Kuivaniemi H, Tromp G, Prockop D J. Hum Mutat. 9(4):300-15 (1997)
39. Cho J Y, et al., Proc Natl Acad Sci USA. (2004)
40. Kapadia R M, et al., Dev Biol.; 285(2):496-507. (2005)
41. Ohbayashi N et al., 16(7) Genes Dev. (2002)
42. Naski M C, Colvin J S, Coffin J D, Ornitz D M Development.; 125(24) (1998).
43. Kang R, Zeh H J, Lotze M T, Tang D. Cell Death Differ. 18(4):571-80. (2011)
44. Alvarez-Garcia O, et a I., Pediatr Nephrol. 22(7):954-61 (2007)
45. Gonzalez D I, et al., Pediatr Nephrol. 26(6):961-6. (2011)
46. Settembre C, et al., Autophagy. 5(2):228-9. (2009)
47. Russell R C, et al., Nat Cell Biol.; 15(7):741-50.(2013)
48. Clarke L. A., Hollak C. E. M. Best Practice & Research Clinical Endocrinology & Metabolism 29 (2015) 219e235
49. Pattni K, et al., Curr Biol. October 16; 11(20):1636-42 (2001)
50. Ferla R, et al., Hum Gene Ther. 2014 July; 25(7):609-18.
51. Stevenson D A, Steiner R D Pediatr Endocrinol Rev. 2013 June; 10 Suppl 2:406-16.
52. Billard C Mol Cancer Ther. 2013 September; 12(9):1691-700.
53. Settembre, C. et al. EMBO J. 31, 1095-1108 (2012).
54. Zhang, Y. et al. Nature 513, 440-443 (2014).
55. Laplante, M. & Sabatini, D. M. Cell 149, 274-293 (2012).
56. Zhang, Y. & Manning, B. D. Cell Cycle Georget. Tex 14, 2011-2017 (2015).
57. Efeyan, A., Zoncu, R. & Sabatini, D. M. Trends Mol. Med. 18, 524-533 (2012).
58. Tomatsu, S. et al. Proc. Natl. Acad. Sci. U.S.A 99, 14982-14987 (2002).
59. Evers, M. et al. Proc. Natl. Acad. Sci. U.S.A 93, 8214-8219 (1996).
60. Pievani, A. et al. Cytotherapy 16, 893-905 (2014).
61. Wraith, J. E., Rogers, J. G. & Danks, D. M. Aust. Paediatr. J. 23, 329-334 (1987).
62. Sardiello, M et al. Science 325, 473-477 (2009).
63. Backer, J. M. Biochem. J. 410, 1-17 (2008).

1. A method for the treatment and/or prevention of a bone growth disorder in a subject in need thereof comprising administering an effective amount of an activator of beclin 1-Vps 34 complex wherein said activator is selected from the group consisting of:
a) a polypeptide comprising a Beclin 1 peptide consisting of SEQ ID No. 43 or a functional fragment thereof or a functional derivative thereof;
b) a polynucleotide coding for said polypeptide;
c) a vector comprising said polynucleotide;
d) a host cell expressing said polypeptide or said polynucleotide; and
e) a small molecule selected from the group of a mTORC1 inhibitor or a BH3 mimetic.

2. The method according to claim 1 wherein said activator increases phosphatidylinositol 3-phosphates (PI3P) production in a cell.

3. The method according to claim 1, wherein the functional fragment comprises residues 270-278 of SEQ ID No. 43.

4. The method according to claim 3, wherein the functional fragment is flanked by no more than twelve naturally-flanking Beclin 1 residues.

5. The method according to claim 1, wherein the functional derivative comprises SEQ ID NO: 43 or a functional fragment thereof and wherein said functional derivative comprises from 1 to 6 amino acid residue substitution(s) and/or a heterologous moiety.

6. The method according to claim 5 wherein the heterologous moiety consists of SEQ ID No. 44 or SEQ ID No. 45.

7. The method according to claim 1, wherein the polypeptide or the functional fragment thereof or the functional derivative thereof is partially or fully cyclized.

8. The method according to claim 1 wherein the polypeptide is a retro-inverso polypeptide.

9. The method according to claim 1, wherein the polypeptide comprises a sequence selected from the group consisting of: SEQ ID No. 1, SEQ ID No. 2, SEQ ID No. 12 to SEQ ID No. 38 or a functional fragment thereof or a functional derivative thereof.

10. The method according to claim 1, wherein the polynucleotide comprises SEQ ID NO:7.

11. The method according to claim 1, wherein said vector is a viral vector.

12. The method according to claim 1 further comprising a polynucleotide coding for the wild-type form of the protein whose mutated form is responsible for the bone growth disorder or a vector comprising said polynucleotide, or further comprising the wild-type form of a protein whose mutated form is responsible for the bone growth disorder.

13. The method according to claim 12 wherein the protein whose mutated form is responsible for the bone growth disorder is selected from the group consisting of: FGFR3, FGFR1, FGFR2, FGFR4, β-glucocerebrosidase, α-mannosidase, α-fucosidase, α-neuraminidase, Cathepsin-A, UDP-N-acetylglucosamine, N-acetylglucosamine-1-phosphotransferase, Sulfatase modifying factor 1, Cathepsin K, α-L-iduronidase, Iduronate-2-sulfatase, Heparan N-sulfatase, α-N-acetyl glucosaminidase, Acetyl-CoA: α-glucosaminide acetyltransferase, N-acetylglucosamine 6-sulfatase, N-acetylgalactosamine-6-sulfatase, β-D-galactosidase, N-acetylgalactosamine-4-sulfatase, β-glucuronidase and Hyaluronidase.

14. The method according to claim 1 wherein the inhibitor of mTORC1 is selected from the group consisting of: Rapamycin, KU0063794, WYE354, Deforolimus, TORIN 1, TORIN 2, Temsirolimus, Everolimus, sirolimus, NVP-BEZ235 and PI103.

15. The method according to claim 1 wherein the bone growth disorder is selected from the group consisting of: achondroplasia, hypochondroplasia, spondyloepiphyseal dysplasia, a lysosomal storage disorder, preferably a mucopolysaccharidosis (MPS).

16. The method according to claim 15 wherein the lysosomal storage disorder is selected from the group consisting of: MPS I, MPS II, MPS IV, MPS VI, MPS VII, MPS IX, Gaucher disease type 3, Gaucher disease type 1, multiple sulfatase deficiency, mucolipidosis type II, mucolipidosis type III, galactosidosis, alpha-mannosidosis, beta-mannosidosis, fucosidosis and pycnodysostosis.

17. The method according to claim 1 wherein the bone growth disorder is selected from the group consisting of: achondroplasia, MPS VI MPS VII.

18. (canceled)

19. (canceled)

20. The method according to claim 1 further comprising administering a therapeutic agent is selected from the group consisting of: enzyme replacement therapy, growth hormone and BMN111.

21. (canceled)

22. The method according to claim 1, wherein said vector comprises a polynucleotide coding for an activator of beclin 1-Vps 34 complex, wherein said activator of beclin 1-Vps 34 complex is a polypeptide comprising a Beclin 1 peptide consisting of SEQ ID No. 43 or a functional fragment thereof or a functional derivative thereof, preferably, the functional fragment comprises residues 270-278 of SEQ ID No. 43, preferably the functional derivative comprises SEQ ID NO: 43 or a functional fragment thereof and said functional derivative comprises from 1 to 6 amino acid residue substitution(s) and/or a heterologous moiety.

23. The method according to claim 22 wherein the polynucleotide encodes a peptide consisting of a sequence selected from the group consisting of: SEQ ID No. 1, SEQ ID No. 2, SEQ ID No. 12 to SEQ ID No. 38 or a functional fragment thereof or a functional derivative thereof.

24. The method according to claim 23, wherein the polynucleotide comprises SEQ ID No. 3.

25. The method according to claim 22, wherein the vector is a viral vector, and optionally an adeno-associated vector (AAV).

26. The method according to claim 22, further comprising a polynucleotide coding for the wild-type form of the protein whose mutated form is responsible for a bone growth disorder.


SEQUENCE LISTING (47 sequences)

SEQ ID NO: 1 - protein, 31 aa, Artificial Sequence
  synthetic sequence
YGRKKRRQRRRGGTNVFNATFEIWHDGEFGT

SEQ ID NO: 2 - protein, 31 aa, Artificial Sequence
  synthetic sequence
RRRQRRKKRGYGGTGFEGDHWIEFTANFVNT

SEQ ID NO: 3 - DNA, 2217 bp, Artificial Sequence
  synthetic sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtcagctactgggacaccggggtcctgctgtgcgcgctgctcagctgtctgcttctcacaggatctagttcaggttacggccggaagaagcggcggcagcggcggcggggcggcaccaacgtgttcaacgccaccttccacatctggcacagcggccagttcggcaccggatccgactacaaagaccatgacggtgattataaagatcatgacatcgactacaaggatgacgatgacaagtgaaagcttaaaaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - DNA, 130 bp, Artificial Sequence
  synthetic sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 5 - DNA, 753 bp, Artificial Sequence
  synthetic sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 6 - DNA, 78 bp, Artificial Sequence
  synthetic sequence
atggtcagctactgggacaccggggtcctgctgtgcgcgctgctcagctgtctgcttctcacaggatctagttcaggt

SEQ ID NO: 7 - DNA, 93 bp, Artificial Sequence
  synthetic sequence
tacggccggaagaagcggcggcagcggcggcggggcggcaccaacgtgttcaacgccaccttccacatctggcacagcggccagttcggcacc

SEQ ID NO: 8 - DNA, 66 bp, Artificial Sequence
  synthetic sequence
gactacaaagaccatgacggtgattataaagatcatgacatcgactacaaggatgacgatgacaag

SEQ ID NO: 9 - DNA, 542 bp, Artificial Sequence
  synthetic sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 10 - DNA, 215 bp, Artificial Sequence
  synthetic sequence
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 11 - DNA, 130 bp, Artificial Sequence
  synthetic sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - protein, 24 aa, Artificial Sequence
  synthetic sequence
RRQRRKKKRGYGGDHWIEFTANFV

SEQ ID NO: 13 - protein, 15 aa, Artificial Sequence
  synthetic sequence
VFNATFHIWHSGQFG

SEQ ID NO: 14 - protein, 18 aa, Artificial Sequence
  synthetic sequence
TNVFNATFHIWHSGQFGT

SEQ ID NO: 15 - protein, 15 aa, Artificial Sequence
  synthetic sequence
VFNATFEIWHDGEFG

SEQ ID NO: 16 - protein, 9 aa, Artificial Sequence
  synthetic sequence
FNATFHIWH

SEQ ID NO: 17 - protein, 11 aa, Artificial Sequence
  synthetic sequence
VFNATFEIWHD

SEQ ID NO: 18 - protein, 11 aa, Artificial Sequence
  synthetic sequence
CFNATFEIWHD

SEQ ID NO: 19 - protein, 11 aa, Artificial Sequence
  synthetic sequence
VWNATFEIWHD

SEQ ID NO: 20 - protein, 11 aa, Artificial Sequence
  synthetic sequence
VFNATFDIWHD

SEQ ID NO: 21 - protein, 11 aa, Artificial Sequence
  synthetic sequence
VFNATFELWHD

SEQ ID NO: 22 - protein, 11 aa, Artificial Sequence
  synthetic sequence
VFNATFEIFHD

SEQ ID NO: 23 - protein, 11 aa, Artificial Sequence
  synthetic sequence
VFNATFEIWYD

SEQ ID NO: 24 - protein, 11 aa, Artificial Sequence
  synthetic sequence
VFNATFEIWHE

SEQ ID NO: 25 - protein, 11 aa, Artificial Sequence
  synthetic sequence
VWNATFELWHD

SEQ ID NO: 26 - protein, 11 aa, Artificial Sequence
  synthetic sequence
VFNATFEVWHD

SEQ ID NO: 27 - protein, 11 aa, Artificial Sequence
  synthetic sequence
VLNATFEIWHD

SEQ ID NO: 28 - protein, 11 aa, Artificial Sequence
  synthetic sequence
VFNATFEMWHD

SEQ ID NO: 29 - protein, 11 aa, Artificial Sequence
  synthetic sequence
VWNATFHIWHD

SEQ ID NO: 30 - protein, 11 aa, Artificial Sequence
  synthetic sequence
VFNATFEFWHD

SEQ ID NO: 31 - protein, 11 aa, Artificial Sequence
  synthetic sequence
VFNATFEYWHD

SEQ ID NO: 32 - protein, 11 aa, Artificial Sequence
  synthetic sequence
VFNATFERWHD

SEQ ID NO: 33 - protein, 10 aa, Artificial Sequence
  synthetic sequence
FNATFEIWHD

SEQ ID NO: 34 - protein, 10 aa, Artificial Sequence
  synthetic sequence
VFNATFEIWH

SEQ ID NO: 35 - protein, 9 aa, Artificial Sequence
  synthetic sequence
FNATFEIWH

SEQ ID NO: 36 - protein, 9 aa, Artificial Sequence
  synthetic sequence
WNATFHIWH

SEQ ID NO: 37 - protein, 10 aa, Artificial Sequence
  synthetic sequence
VWNATFHIWH

SEQ ID NO: 38 - protein, 10 aa, Artificial Sequence
  synthetic sequence
WNATFHIWHD

SEQ ID NO: 39 - DNA, 583 bp, Artificial Sequence
  synthetic sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgt

SEQ ID NO: 40 - DNA, 709 bp, Homo sapiens
gctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcag

SEQ ID NO: 41 - DNA, 840 bp, Artificial Sequence
  synthetic sequence
caccttcacacaggtctccttctgtgcagtaacacaccagctcttttcctggctgtcggctcaggccaacttcggcctgtgctccagaggaagccttcaacgcagagctggatgggggaggggtggagggcagtcgctgtgaacgtccaggtgggagtctggggaccaggtactgcagggaagggctaaaagataggtcggggtaacccttcagatctggctcagctagcctgtctccaagatttaggactctgaatctctgtgggctcctccctgtccccactcccaaacgcctgacgcggtgccccctcgccctccgctgctcctttctaccgctttccctcctccctcccatgtcttttccgtccttggtctagggctctcggcctgcgcctctgcaaacaccccctcccctccaactccggcagaactccgaggggaggggccggaggccacccttcccgcctgtggtcagaggggggcagcgccgcagccccgggtttggggggcaggggccatctctgcgccccgcccgatcaggccactcggcgcactaggggtggagggcgggaagcgtgactcccagagaggggggtccggcttgggcaggtgcgggcactggcagggcccaggcgggctccgggggcgggcggttcaggttacagcccagcggggggcagggggcggcccgcggtttgggcgagttcgccagcctcgaaaggggccgggcgcatataacgggcgccgcggcggggagaagacgcagagcgctgctgggctgccgggtctcccgcttccccctcctgctccaagggcctcctgcatgagggcgcggtagag

SEQ ID NO: 42 - DNA, 952 bp, Artificial Sequence
  synthetic sequence
gcttcttgatccaactgagaaggaaaaaggagcccagcaagaagagggggagagagagaaggggaaaggggggaacccaccagcaccctccgtcggactcttgaagccttttttttttaattcttaatttttttttttactctttacaaaaagtaaagtgagaatcctgctctctaatacatctgcaagacatcaccctctcctcctgaaactttagtcactcctgagaatccacaggagtgcagagaggggggaacacgttttcttgaagatgttttaaagctggaacaagccttcttctgttggtgcttgaactcttgcctgggaataacttttttaacctttaaaaaaaccattcactttgattcttctctcccaccccttcttctctcttcttctgtttgcctaactcccccgccctgctggcctccgctttcctctctcccccttgttattatttttagtctgtgcgtgtggacacttttggagagttggaagggatttttttctcctgacttgaacatagggtgactttttaatattgtattttactgtggattatctctttggaccgcgccggacttggcctcaggaaatcaaccaatgctgcggaaggcggctggtgcacaacgctctgctctacagaagggggtcccccaccctcttttccaattttttttttttggccttcctctccttccctccctcttcctccctctctctctctctctctccactacccccctctttcttccccactcggctcctctcccccctcgcgcccacagcgtttggtgttgattcgagcgggaagaggggggtgggtgggatcggtgggggagaccatgacctccagctacgggcacgttctggagcggcaaccggcgctgggcggccgcttggacagcccgggcaacctcgacaccctgcaggcgaaaaagaacttctccgt

SEQ ID NO: 43 - protein, 450 aa, Artificial Sequence
  synthetic sequence
MEGSKTSNNSTMQVSFVCQRCSQPLKLDTSFKILDRVTIQELTAPLLTTAQAKPGETQEEETNSGEEPFIETPRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQMELKELALEEERLIQELEDVEKNRKIVAENLEKVQAEAERLDQEEAQYQREYSEFKRQQLELDDELKSVENQMRYAQTQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 44 - protein, 11 aa, Artificial Sequence
  synthetic sequence
YGRKKRRQRRR

SEQ ID NO: 45 - protein, 11 aa, Artificial Sequence
  synthetic sequence
RRRQRRKKRGY

SEQ ID NO: 46 - protein, 15 aa, Artificial Sequence
  synthetic sequence
GFQGSHWIHFTANFV

SEQ ID NO: 47 - protein, 7 aa, Artificial Sequence
  synthetic sequence
SMSIARL